(19) United States
(12) Patent Application Publication
(10) Pub. No.: US 2026/0139270 A1
(43) Pub. Date: May 21, 2026

(54) HYBRID AAV VECTOR ENHANCING TRANSGENE EXPRESSION IN THE LIVER

(71) Applicants: GENETHON, Evry-Courcouronnes (FR); INSTITUT NATIONAL DE LA SANTÉ ET DE LA RECHERCHE MÉDICALE, Paris (FR); UNIVERSITE EVRY VAL D'ESSONNE, Evry-Courcouronnes (FR)

(72) Inventors: Tiziana LA BELLA, Maisons-Alfort (FR); Giuseppe RONZITTI, Fontainebleau (FR); Justine SIAUVE, Le Coudray-Montceaux (FR)

(73) Assignees: GENETHON, Evry-Courcouronnes (FR); INSTITUT NATIONAL DE LA SANTÉ ET DE LA RECHERCHE MÉDICALE, Paris (FR); UNIVERSITE EVRY VAL D'ESSONNE, Evry-Courcouronnes (FR)

(21) Appl. No.: 19/119,726

(22) PCT Filed: Oct. 12, 2023
(86) PCT No.: PCT/EP2023/078311

§ 371 Date: Apr. 9, 2025

(30) Foreign Application Priority Data
Oct. 12, 2022 (EP) 22306549.1

(51) Int. Cl. C12N 15/86; A61K 38/44; A61K 38/45; A61K 38/47; A61K 48/00; C07K 14/005; C12N 9/04; C12N 9/10; C12N 9/40

(52) U.S. Cl. CPC C12N 15/86; A61K 38/443; A61K 38/45; A61K 38/47; A61K 48/0058; C07K 14/005; C12N 9/0006; C12N 9/1051; C12N 9/2465; C12Y 101/01049; C12Y 204/01017; C12Y 204/01025; C12Y 302/0102; C12Y 302/01033; C12N 2750/14122; C12N 2750/14143; C12N 2830/008; C12N 2830/50

(57) ABSTRACT

The invention relates to a hybrid AAV vector comprising a transgene of interest operably linked to an hepatocyte-specific promoter, which enhances transgene expression in the liver and its use for liver-directed gene therapy.

### FIELD OF THE INVENTION

The invention relates to a hybrid AAV vector which enhances transgene expression in the liver and its use for liver-directed gene therapy.

### BACKGROUND OF THE INVENTION

Adeno-associated vectors (AAV) are currently the platform of choice in gene therapy for many monogenetic diseases (Valdmanis P N et al., Hum. Gene Ther., 2017, 28, 361-372; Wang D et al., Nat. Rev. Drug Discov., 2019, 18, 358-378).

AAV is a non-pathogenic virus belonging to the genus Dependoparvovirus within the family Parvoviridae. AAV is a non-enveloped virus composed of a capsid of about 26 nm in diameter and a single-stranded DNA genome of 4.7 kb. The genome carries two genes, rep and cap, flanked by two palindromic regions named Inverted terminal Repeats (ITR) that serve as the viral origins of replication and the packaging signal. The cap gene codes for three structural proteins VP1, VP2 and VP3 that compose the AAV capsid through alternative splicing and translation from different start codons. VP1, VP2 and VP3 share the same C-terminal end which is all of VP3. Using AAV2 has a reference, VP1 has a 735 amino acid sequence (GenBank accession number YP_680426.1 accessed on 13 Aug. 2018); VP2 (598 amino acids) starts at the Threonine 138 (T138) and VP3 (533 amino acids) starts at the methionine 203 (M203). The rep gene encodes four proteins required for viral replication Rep78, Rep68, Rep52 and Rep40. Recombinant AAV vectors encapsidate an ITR-flanked rAAV genome in which a therapeutic gene expression cassette replaces the AAV protein coding-sequences.

One of the key advantages is their ability to naturally infect human cells and persist at long term in non-dividing cells. Moreover, the AAV capsid plays a key role in the tropism by interacting with the host cell receptors thus determining the tissue specificity of the targeting. With the increased number of gene transfer clinical trials targeting the liver, it is crucial to develop new AAV vectors with better expression of the therapeutic gene in the liver.

### SUMMARY OF THE INVENTION

The inventors have combined AAV hybrid capsids designed according to the rational shuffling strategy previously disclosed (WO 2022/003211) with an hepatocyte-specific promoter. An increase of transgene expression in the liver was observed as illustrated for some AAV hybrid capsids in FIG. 1 and some hepatocyte-specific promoters in FIG. 4. Surprisingly, no variation in the vector genome copy number per diploid cells were detected, indicating a direct role of the AAV hybrid capsid on the transgene expression itself (FIG. 2). No enhancement of transgene expression by the AAV hybrid capsids was observed using a ubiquitous promoter, demonstrating that the AAV hybrid capsid enhances the effect of the hepatocyte-specific promoter on transgene expression (FIG. 3).

The invention relates to a hybrid adeno-associated virus (AAV) vector, comprising a recombinant hybrid AAV capsid protein and a transgene of interest under the control of an hepatocyte-specific promoter, wherein the capsid protein is a hybrid between at least two recombinant AAV capsid proteins from different AAV serotypes, an acceptor AAV capsid protein and at least one donor AAV capsid protein, said hybrid AAV capsid protein comprising at least one hypervariable region (HVR) sequence from the donor AAV capsid protein(s) selected among the HVR1, HVR5, HVR7 or HVR10 sequence, replacing the corresponding HVR sequence(s) of the acceptor AAV capsid protein.

In particular embodiments, the donor AAV capsid serotype is AAV2, AAV13 or hybrid AAV2/13; preferably the hybrid AAV2/13 capsid is selected from the group consisting of the sequences SEQ ID NO: 2 to 30; and/or wherein the acceptor AAV capsid serotype is selected from the group consisting of: AAV8, AAV9, AAV5, AAV-LK03, AAVrh74, AAV9.rh74, and AAVrh10; preferably AAV8 or AAV9.

In particular embodiments, the HVR1 sequence from the donor AAV capsid protein consists of:

EX₁X₂KTAPGKKRX₃VX₄X₅X₆X₇X₈EPDSSSGX₉GKX₁₀GX₁₁,

wherein X₁ is P, H, A, preferably P; X₂ is V or A, preferably V; X₃ is P or A, preferably P; X₄ is E or A, preferably E; X₅ is H or Q, preferably H; X₆ is S or A, preferably S; X₇ is P or H, preferably P; X₈ is V or A, preferably V; X₉ is I or T, preferably T; X₁₀ is A or S, preferably A; and X₁₁ is Q, K or N, preferably Q; preferably the HVR1 sequence from the donor AAV capsid is selected from the group consisting of: SEQ ID NO: 33 to 40; more preferably SEQ ID NO: 33.

In particular embodiments, the HVR5 sequence from the donor AAV capsid protein consists of:

YYLX₁X₂TX₃X₄X₅SGTX₆X₇X₈SRLX₉FSQAGX₁₀X₁₁X₁₂X₁₃X₁₄
X₁₅X₁₆X₁₇X₁₈NWLPGP,

wherein X₁ is N or S, preferably N; X₂ is K or R, preferably K; X₃ is Q or N, preferably Q; X₄ is T, S or A, preferably S; X₅ is P, T, N or A; X₆ is L, P, T, V or is absent; X₇ is Q or T; X₈ is Q or M, preferably Q; X₉ is Q or L, preferably L; X₁₀ is P or A, preferably P; X₁₁ is T or S, preferably T; X₁₂ is S or D, preferably S; X₁₃ is M or I, preferably M; X₁₄ is S or R, preferably S; X₁₅ is L or D, preferably L; X₁₆ is Q or H, preferably Q; X₁₇ is A or S, preferably A; and X₁₈ is K or R, preferably K; preferably the HVR5 sequence from the donor AAV capsid is selected from the group consisting of: SEQ ID NO: 41 to 49; more preferably SEQ ID NO: 41.

In particular embodiments, the HVR7 sequence from the donor AAV capsid protein consists of:

SX₁X₂X₃WTX₄ATKYH,

wherein X₁ is D, E or N, preferably N; X₂ is Y or F, preferably F; X₃ is S or P, preferably P; X₄ is G or A, preferably A; preferably the HVR7 sequence from the donor AAV capsid is selected from the group consisting of: SEQ ID NO: 50 to 55; more preferably SEQ ID NO: 52.

In particular embodiments, the HVR10 sequence from the donor AAV capsid protein consists of:

TEQYGX₁VSX₂NLQX₃X₄NX₅X₆X₇X₈TX₉X₁₀VNX₁₁QGX₁₂LX₁₃GMV
WQNRD,

wherein X₁ is S, T, A, Y or N, preferably Y; X₂ is T or N, preferably N; X₃ is R, N or S, preferably N; X₄ is G or S, preferably S; X₅ is R, A or T, preferably T; X₆ is Q, G, R or A, preferably G; X₇ is A or P, preferably P; X₈ is A, T or S, preferably T; X₉ is A, S, G or E, preferably G; X₁₀ is T, D or N, preferably T; X₁₁ is T, A, H or N, preferably H; X₁₂ is A, I or V, preferably A; and X₁₃ is P or S, preferably P; preferably the HVR10 sequence from the donor AAV capsid is selected from the group consisting of: SEQ ID NO: 56 to 64; more preferably SEQ ID NO: 58.

In preferred embodiments, the recombinant hybrid AAV capsid protein comprises or consists of a sequence selected from the group consisting of the sequences SEQ ID NO:65, 68, 71, 74, 77, 80, 83, 86, 89, 95, 101 and the sequences having at least 85%, 90%, 95%, 97%, 98% or 99% identity with said sequences.

In some embodiments, the hepatocyte-specific promoter is selected from the group comprising: an alpha-1 antitrypsin (AAT) promoter, a transthyretin (TTR) promoter, an albumin (ALB) promoter, a thyroxine-binding globulin (TBG) promoter, and an HBV core promoter; preferably human alpha-1 antitrypsin (hAAT) promoter of SEQ ID NO: 107, LP1 promoter of SEQ ID NO: 117, HLP promoter, SERPINA1_COMP_D promoter of SEQ ID NO: 121, TTR promoter of SEQ ID NO: 119, HSE promoter of SEQ ID NO: 120, TBG full-length promoter, TBG minimal promoter, and TBG-derived promoter of SEQ ID NO: 118.

In particular embodiments, the hepatocyte-specific promoter is associated to an enhancer sequence capable of enhancing hepatocyte-specific expression of genes, preferably the ApoE control region, more preferably human ApoE control region.

In particular embodiments, the transgene expression cassette comprises, in the 5′ to 3′ orientation, an hepatocyte-specific promoter, preferably hAAT promoter; the transgene coding sequence; and a polyadenylation signal such as (bGH) polyA; more preferably additionally comprising one or more further regulatory elements chosen from an enhancer, preferably human ApoE control region and an intron, preferably HBB2 intron or hFIX intron.

In particular embodiments, the transgene of interest is selected from the group consisting of: therapeutic genes; genes encoding therapeutic proteins or peptides such as therapeutic antibodies or antibody fragments and genome editing enzymes; and genes encoding therapeutic RNAs such as interfering RNAs, guide RNAs for genome editing and antisense RNAs capable of exon skipping.

In particular embodiments, the transgene encodes a protein with a therapeutic effect on hepatocytes or a therapeutic protein that is secreted from the liver into the bloodstream; preferably selected from the group consisting of: Acid Alpha-Glucosidase (GAA), FGF23 antagonist, Amylo-Alpha-1,6-Glucosidase, 4-Alpha-Glucanotransferase (AGL), Glucose-6-phosphate dehydrogenase (G6PD) and Uridine Diphosphate Glucuronosyltransferase isoform 1A1 (UGT1A1).

Another aspect of the invention relates to pharmaceutical composition comprising a therapeutically effective amount of hybrid AAV vector according to the present disclosure or hepatocyte stably transduced by said hybrid AAV vector.

Another aspect of the invention relates to the hybrid AAV vector, hepatocyte, pharmaceutical composition according to the present disclosure, for use as a medicament for liver-targeted gene therapy.

Another aspect of the invention relates to the hybrid AAV vector, hepatocyte, pharmaceutical composition of the present disclosure for use in the treatment of a genetic disease selected from the group consisting of: Hemophilia A, Hemophilia B, X-linked hypophosphatemia, Crigler-Najjar syndrome, Glucose-6-phosphate dehydrogenase deficiency, and Glycogen storage diseases types I, II and III.

### DETAILED DESCRIPTION OF THE INVENTION

The invention relates to a hybrid adeno-associated virus (AAV) vector that enhances transgene expression in the liver and its use for liver-directed gene therapy. The hybrid AAV vector according to the invention is a recombinant AAV (rAAV) vector particle comprising a recombinant hybrid AAV capsid protein and a transgene of interest under the control of an hepatocyte-specific promoter.

### AAV Particle, Cell

### Hybrid AAV Capsids

The recombinant hybrid AAV capsid protein is a hybrid between at least two recombinant AAV capsid proteins from different AAV serotypes, an acceptor AAV capsid protein and at least one donor AAV capsid protein. The hybrid AAV capsid comprises at least one hypervariable region (HVR) sequence from the donor AAV capsid protein(s) chosen from HVR1, HVR5, HVR7 and HVR10 (replacement HVR sequence(s)) replacing the corresponding HVR sequence(s) of the acceptor AAV capsid protein (replaced HVR sequence(s)); the replacement HVR sequence(s)) have by definition an amino acid sequence which is different from that of the replaced HVR sequence(s). The recombinant hybrid AAV capsid protein may be derived from any different natural or artificial AAV serotypes used as acceptor and donor AAV capsid serotypes such as in particular those described in the present disclosure.

As used herein “AAV serotype” or “AAV capsid serotype” refers to an AAV capsid having distinct hypervariable region (HVR) amino acid sequences compared to an AAV capsid of another serotype. Different AAV serotypes have amino acid variation in their HVR sequences. The term AAV serotype encompasses any natural or artificial AAV capsid serotype including AAV capsid variants isolated from primate (human or non-human) or non-primate species and AAV capsid variants engineered by various techniques known in the art such as for example rational design, directed evolution and in silico discovery. As used herein, the term AAV serotype refers to a functional AAV capsid which is able to form recombinant AAV viral particles which transduce a cell, tissue or organ, in particular a cell tissue or organ of interest (target cell, tissue or organ) and express a transgene in said cell, tissue or organ, in particular target cell tissue or organ, e.g., hepatocyte and derived liver tissue or organ.

As used herein “hybrid AAV capsid”, “AAV hybrid capsid”, “AAV hybrid serotype”, “hybrid AAV serotype”, “hybrid capsid” or “hybrid serotype” refers to the recombinant hybrid AAV capsid protein according to the present disclosure.

As used herein, “AAV vector”, “AAV particle”, “AAV vector particle”, “rAAV vector”, “rAAV particle”, “rAAV vector particle”, “rAAV”, “rAAV viral vector”, “vector”, “AAV vector serotype” refers to the hybrid AAV vector according to the invention.

As used herein, “hypervariable region or HVR” refers to any one of HVR1 to HVR12 of an AAV capsid. According to a narrow definition of HVR, HVR1 is from positions 146 to 153; HVR2 is from positions 183-187; HVR3 is from positions 263 to 267; HVR4 is from positions 384 to 386; HVR5 is from positions 453 to 477; HVR6 is from positions 493 to 498; HVR7 is from positions 503 to 507; HVR8 is from positions 517 to 525; HVR9 is from positions 536 to 559; HVR10 is from positions 584 to 597; HVR11 is from positions 661 to 670; and HVR12 is from positions 708 to 722; the indicated positions being determined by alignment with SEQ ID NO: 1 (VP1 of AAV8 or AAV8 capsid). After sequence alignment of any other AAV capsid sequence of any other serotype with SEQ ID NO: 1 using standard protein sequence alignment programs that are well-known in the art, such as for example BLAST, FASTA, CLUSTALW, MEGA and the like, a person skilled in the art can easily obtained the corresponding positions of the hypervariable regions in other AAV capsid serotypes. For example, using MEGA software (version X) with ClustalW alignment algorithm at default parameters, HVR1 to HVR12 are from positions 146 to 152, 182 to 186, 262 to 264, 381 to 383, 450 to 474, 490 to 495, 500 to 504, 514 to 522, 533 to 556, 581 to 594, 658 to 667 and 705 to 719, respectively of the capsid of SEQ ID NO: 2 (named #704).

According to a large definition of HVR, HVR1 is from positions 134 to 165, HVR2 is from positions 176 to 192; HVR3 is from positions 259 to 278; HVR4 is from positions 379 to 395; HVR5 is from positions 446 to 485; HVR6 is from positions 485 to 502; HVR7 is from positions 499 to 516; HVR8 is from positions 509 to 531; HVR9 is from positions 531 to 570; HVR10 is from positions 576 to 613; HVR11 is from positions 621 to 687; and HVR12 is positions 687 to 738; preferably HVR1 is from positions 134 to 165, HVR2 is from positions 176 to 192; HVR3 is from positions 259 to 278; HVR4 is from positions 379 to 395; HVR5 is from positions 446 to 484; HVR6 is from positions 490 to 500; HVR7 is from positions 501 to 512; HVR8 is from positions 514 to 529; HVR9 is from positions 531 to 570; HVR10 is from positions 576 to 613; HVR11 is from positions 630 to 682; and HVR12 is from positions 705 to 736; the indicated positions being determined by alignment with SEQ ID NO: 1 (VP1 of AAV8 or AAV8 capsid).

The positions of the HVR sequence from the donor or acceptor AAV capsids may differ from the positions indicated above (HVR reference sequence) by few amino acids. Depending on the initial size of the HVR and the distance between the different HVRs, both HVR sequences (replaced sequence from the acceptor capsid and replacement sequence from the donor capsid(s)) consist of at least 2 amino acids to about 70 amino acids. For example the HVR sequence from the donor or acceptor AAV capsids may have a deletion of 1 amino acid at one or both ends of a HVR sequence of up to 5 amino acids; a deletion of up to 2 amino acids (1 or 2 amino acids) at one or both ends of a HVR sequence of 6 to 10 amino acids; a deletion of up to 5 amino acids (1, 2, 3, 4 or 5 amino acids) at one or both ends of a HVR sequence of 11 to 25 amino acids. Alternatively, the HVR sequence from the donor or acceptor AAV capsid may have additional sequence from the N- or C-terminus of the HVR sequence, for example up to 10, 20, 30, 40 or 50 amino acids from the N- or C-terminus of the HVR sequence. Preferably, the amino acid deletion or addition at one or both ends of the HVR sequence involves consecutive amino acids from the donor or acceptor AAV capsid sequence.

The hybrid AAV capsid protein is a functional AAV capsid which is able to form recombinant AAV viral particles which transduce a cell, tissue or organ, in particular a cell tissue or organ of interest (target cell, tissue or organ) and express a transgene in said cell, tissue or organ, in particular target cell tissue or organ, e.g., hepatocyte and derived liver tissue or organ. Furthermore, the hybrid AAV vector according to the invention which comprises the hybrid capsid protein and an hepatocyte-specific promoter enhances transgene expression in the liver compared to an hybrid AAV vector comprising the hybrid capsid protein and an ubiquitous promoter and to an AAV vector comprising the parent acceptor AAV capsid protein and an hepatocyte-specific promoter. An increased transgene expression in the liver refers in particular to a transgene expression level that is increased by at least 1.1 fold, preferably 1.2, 1.3, 1.4, 1.4, 1.5, 1.6, 1.7, 1.8, 1.9, 2, 2.5, 3 folds compared to parent acceptor AAV capsid protein. The ability of the hybrid AAV capsid protein to enhance transgene expression in the liver may be determined using standard assays that are well-known in the art such as those disclosed in the examples of the present application. For example, transgene expression in the liver is determined by systemic administration of hybrid AAV vector particles in animal models such as mouse models that are well known in the art and disclosed in the examples of the present application. Parent AAV vector serotype comprising the acceptor capsid is used for comparison. Transgene expression is advantageously measured using a reporter gene such as luciferase or fluorescent protein (GFP or others) by standard assays that are well known in the art such as in vivo or in vitro quantitative bioluminescence or fluorescence assays in vivo or in vitro. Transgene expression may also be advantageously measured using a gene encoding a secreted protein that is expressed in the liver and secreted from the liver into the bloodstream. The level of secreted protein in the serum may be measured by standard assays that are well known in the art such as ELISA.

The liver consists of several cell types classified into hepatocytes (liver parenchymal cells) that constitute about 80% of liver cells and non-parenchymal cells represented by endothelial cells, Kupffer cells (resident liver macrophages), fat-storing cells (stellate cells or Ito cells), and pit cells (natural killer cells). Kupffer and endothelial cells form the hepatic reticulo-endothelial system and constitute the majority of liver non-parenchymal cell types. As used herein “liver cell” consists of hepatocyte. As used herein, “hepatocyte” includes primary hepatocyte such as from adult or fetal liver; hepatocyte matured in vitro, hepatocyte cell line; hepatic progenitor or pluripotent stem cell such as induced pluripotent stem cell (iPS cell), embryonic stem cells, fetal stem cell and adult stem cell.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

In the following description, the residues are designated by the standard one letter amino acid code and the indicated positions are determined by alignment with SEQ ID NO: 1.

The term “identity” refers to the sequence similarity between two polypeptide molecules or between two nucleic acid molecules. When a position in both compared sequences is occupied by the same base or same amino acid residue, then the respective molecules are identical at that position. The percentage of identity between two sequences corresponds to the number of matching positions shared by the two sequences divided by the number of positions compared and multiplied by 100. Generally, a comparison is made when two sequences are aligned to give maximum identity. The identity may be calculated by alignment using, for example, the GCG (Genetics Computer Group, Program Manual for the GCG Package, Version 7, Madison, Wisconsin) pileup program, or any of sequence comparison algorithms such as BLAST, FASTA or CLUSTALW.

The acceptor and donor AAV capsids may be from any different natural or artificial AAV serotypes. At least 13 different AAV serotypes (AAV1 to 13) have been identified in human and non-human primates and classified in various clades and clones based on phylogenetic analysis of VP1 sequences of various primate AAV isolates: AAV1 and AAV6 correspond to Clade A; AAV2 to Clade B; AAV2-AAV3 hybrid to Clade C; AAV7 to Clade D; AAV8 to Clade E; AAV9 to Clade F, whereas AAV3, AAV4 and AAV5 are disclosed as clones (Gao et al., J. Virol., 2004, 78, 6381-6388). AAV2 variant serotypes and AAV2/13 hybrid capsids have been isolated in human liver (La Bella et al., Gut, 2020, 69, 737-747.doi: 10.1136/gutjnk-2019-318281 and WO2020/216861; SEQ ID NO: 2 to 30 in the attached sequence listing). Other AAV serotypes have been identified in non-primate species, such as porcine, bovine, avian and caprine. Porcine AAV includes in particular AAVpo1, po2.1, po4 to 6. Various AAV capsid variants, also named “synthetic AAV serotypes” or new AAV serotypes” have been engineered, in particular by directed gene evolution or in silico discovery such as with no limitations recombinant AAV2-derived serotypes DJ, DJ8 and PHP.B which are hybrid capsids from 8 AAV serotypes (AAV2, 4, 5, 8, 9, avian, bovine and goat) AAV-Anc80, AAV2i8, AAV-LK03 and others.

In some embodiments, the acceptor AAV capsid protein is from an AAV serotype used in gene therapy, also named “conventional AAV serotype” such as for example AAV1, AAV2, AAV2 variants (such as the quadruple-mutant capsid optimized AAV2 comprising an engineered capsid with Y44+500+730F+T491V changes, disclosed in Ling et al., 2016 Jul. 18, Hum Gene Ther Methods.), AAV3 and AAV3 variants (such as the AAV3-ST variant comprising an engineered AAV3 capsid with two amino acid changes, S663V+T492V, disclosed in Vercauteren et al., 2016, Mol. Ther. Vol. 24(6), p. 1042),-3B and AAV-3B variants, AAV4, AAV5, AAV6 and AAV6 variants (such as the AAV6 variant comprising the triply mutated AAV6 capsid Y731F/Y705F/T492V form disclosed in Rosario et al., 2016, Mol Ther Methods Clin Dev. 3, p. 16026), AAV7, AAV8, AAV9, AAV 2G9, AAV10 such as AAVcy10 and AAVrh10, AAVrh32.33, AAVrh39, AAVrh43, AAVrh74, AAV-DJ, AAVAnc80, AAV-LK03, AAV.PHP such as AAV-PHP.B, AAV-PHP.EB, AAV2i8, clade F AAVHSC such as AAVHSC7, AAVHSC15 and AAVHSC17, and AAV9.rh74 (WO 2019/193119), porcine AAV such as AAVpo1, AAVpo2.1, AAVpo4 and AAVpo6, and tyrosine, lysine and serine capsid mutants of AAV serotypes. AAV4 capsid (GenBank accession number NC_001829.1); AAV5 capsid (GenBank accession number NC_006152.1 accessed on 13 Aug. 2018); AAV7 capsid (GenBank accession number NC_006260.1); AAV8 (SEQ ID NO: 1); AAV9 capsid (GenBank accession number AY530579.1 accessed on 24 Jun. 2004); AAVrh10 capsid (GenBank accession number AY243015.1 accessed on 14 May 2003); AAV-LK03 (amino acid sequence SEQ ID NO: 31), AAVrh74 and AAV9.rh74 disclosed in WO 2019/193119 (AAVrh74 amino acid sequence SEQ ID NO: 2; AAV9.rh74 amino acid sequence of SEQ ID NO: 3).

In particular embodiments, the acceptor AAV capsid protein is from an AAV serotype selected from the group consisting of: AAV4, AAV5, AAV7, AAV8, AAV9, AAVrh10, AAVrh32.33, AAVrh39, AAVrh43, AAVrh74, AAV9.rh74, AAV-DJ, AAVAnc80, AAV2i8, AAV-LK03, and AAV.PHP. In preferred embodiments, the acceptor AAV capsid serotype is selected from the group consisting of: AAV8, AAV9, AAV5, AAV-LK03, AAVrh74, AAV9.rh74 and AAVrh10; preferably AAV8 or AAV9.

In particular embodiments, the donor AAV capsid protein(s) is from a newly-isolated natural AAV variant serotype such as for example AAV2/13 hybrid serotype, in particular isolated from human tissue such as liver tissue; more preferably selected from the group consisting of the sequences SEQ ID NO: 2 to 30.

In particular embodiments, the donor AAV capsid protein(s) is from an AAV serotype used in gene therapy. The donor AAV capsid protein(s) may be AAV2 or AAV13. AAV13 capsid gene (coding sequence or CDS) sequence corresponds to positions 1948 to 4149 of AAV13 genome sequence GenBank accession number EU285562.1 as accessed on 23 September; AAV13 capsid protein (major coat protein or VP1) amino acid sequence corresponds to GenBank accession number ABZ10812.1 as accessed on 23 Sep. 2008 or SEQ ID NO: 32. AAV2 capsid protein amino acid sequence corresponds to GenBank accession number YP_680426.1 accessed on 13 Aug. 2018.

In particular embodiments, the HVR sequence(s) of the donor AAV capsid protein (replacement HVR sequences) and/or acceptor AAV capsid protein(s) (replaced HVR sequences) are selected from the group consisting of an HVR1 sequence from positions 134 to 165, an HVR5 sequence from positions 446 to 485; an HVR7 sequence from positions 499 to 516; and an HVR10 sequence from positions 576 to 613; preferably an HVR1 sequence from positions 134 to 165, an HVR5 sequence from positions 446 to 484; an HVR7 sequence from positions 501 to 512; and an HVR10 sequence from positions 576 to 613; the indicated positions being determined by alignment with SEQ ID NO: 1 (VP1 of AAV8 or AAV8 capsid). These positions correspond to a large definition of the HVR sequences.

In preferred embodiments, the recombinant hybrid AAV capsid comprises any one of the HVR1, HVR5, HVR7 or HVR10 sequences from the donor AAV capsid replacing the corresponding HVR sequence of the acceptor capsid serotype; e.g., the recombinant hybrid AAV capsid protein comprises the HVR1, HVR5, HVR7 or HVR10 sequence from the donor AAV capsid protein. In more preferred embodiments, the acceptor AAV capsid serotype is selected from the group consisting of: AAV8, AAV9, AAV-LK03, AAVrh74, AAV9.rh74, AAV5 and AAVrh10; preferably AAV8 or AAV9; and/or the donor AAV capsid serotype(s) is selected from the group consisting of AAV13, and the sequences SEQ ID NO: 2 to 30. In more preferred embodiments, the HVR sequence(s) of the donor AAV capsid protein (replacement HVR sequences) and/or acceptor AAV capsid protein(s) (replaced HVR sequences) are selected from the group consisting of an HVR1 sequence from positions 134 to 165, an HVR5 sequence from positions 446 to 484; an HVR7 sequence from positions 499 to 516 or 501 to 512; preferably from positions 501-512; and an HVR10 sequence from positions 576 to 613; the indicated positions being determined by alignment with SEQ ID NO: 1 (VP1 of AAV8 or AAV8 capsid).

In preferred embodiments, the HVR1 sequence from the donor AAV capsid consists of:

EX₁X₂KTAPGKKRX₃VX₄X₅X₆X₇X₈EPDSSSGX₉GKX₁₀GX₁₁,

wherein X₁ is P, H, A, preferably P; X₂ is V or A, preferably V; X₃ is P or A, preferably P; X₄ is E or A, preferably E; X₅ is H or Q, preferably H; X₆ is S or A, preferably S; X₇ is P or H, preferably P; X₈ is V or A, preferably V; X₉ is I or T, preferably T; X₁₀ is A or S, preferably A; and X₁₁ is Q, N or K, preferably Q.

In more preferred embodiments, the HVR1 sequence from the donor AAV capsid is selected from the group consisting of:

(HVR1 of AAV2 and #704; SEQ ID NO: 33)
EPVKTAPGKKRPVEHSPVEPDSSSGTGKAGQ;
(HVR1 of #129; SEQ ID NO: 34)
EPVKTAPGKKRPVEHSPAEPDSSSGTGKAGQ;
(HVR1 of #508; SEQ ID NO: 35)
EPVKTAPGKKRPVAHSPAEPDSSSGTGKAGN;
(HVR1 of #1704; SEQ ID NO: 36)
EPVKTAPGKKRPVEHSPAEPDSSSGTGKSGN;
(HVR1 of #790; SEQ ID NO: 37)
EPVKTAPGKKRPVEHSPVEPDSSSGTGKAGN;
(HVR1 of #2320; SEQ ID NO: 38)
EHVKTAPGKKRAVEHAHVEPDSSSGTGKAGQ;
(HVR1 of AAV13; SEQ ID NO: 39)
EAAKTAPGKKRPVEQSPAEPDSSSGIGKSGQ;
and
(HVR1 of #2112; SEQ ID NO: 40)
EPVKTAPGKKRPVEHSPAEPDSSSGTGKSGK;
preferably SEQ ID NO: 33 (HVR1 of #704).

In preferred embodiments, the HVR5 sequence from the donor AAV capsid consists of:

YYLX₁X₂TX₃X₄X₅SGTX₆X₇X₈SRLX₉FSQAGX₁₀X₁₁X₁₂X₁₃X₁₄
X₁₅X₁₆X₁₇X₁₈NWLPGP,

wherein X₁ is N or S, preferably N; X₂ is K or R, preferably K; X₃ is Q or N, preferably Q; X₄ is T, S or A, preferably S; X₅ is P, T, N or A; X₆ is L, P, T, V or is absent; X₇ is Q or T; X₈ is Q or M, preferably Q; X₉ is Q or L, preferably L; X₁₀ is P or A, preferably P; X₁₁ is T or S, preferably T; X₁₂ is S or D, preferably S; X₁₃ is M or I, preferably M; X₁₄ is S or R, preferably S; X₁₅ is L or D, preferably L; X₁₆ is Q or H, preferably Q; X₁₇ is A or S, preferably A; and X₁₈ is K or R, preferably K.

In more preferred embodiments, the HVR5 sequence from the donor AAV capsid is selected from the group consisting of:

(HVR5 of #667; SEQ ID NO: 41)
-YYLSRTNTPSGTTTQSRLLFSQAGPTSMSLQAKNWLPGP;
(HVR5 of #2731; SEQ ID NO: 42)
-YYLNKTQSNSGTTTMSRLLFSQAGPTSMSLHAKNWLPGP;
(HVR5 of #129; SEQ ID NO: 43)
-YYLNRTQTTSGTPTQSRLLFSQAGPTSMSLQAKNWLPGP;
(HVR5 of #508 and AAV13; SEQ ID NO: 44)
-YYLNRTQTASGTQQSRLLFSQAGPTSMSLQAKNWLPGP;
(HVR5 of #3142; SEQ ID NO: 45)
-YYLNKTQSNSGTVQQSRLLFSQAGPTSMSLQAKNWLPGP;
(HVR5 of #M258; SEQ ID NO: 46)
-YYLNKTQSASGTVQQSRLLFSQAGPTSMSLQAKNWLPGP;
(HVR5 of #1570; SEQ ID NO: 47)
-YYLNKTQANSGTLQQSRLLFSQAGPTSMSLQAKNWLPGP;
(HVR5 of #1602; SEQ ID NO: 48)
-YYLNKTQTNSGTLQQSRLLFSQAGPTSMSLQAKNWLPGP;
and
(HVR5 of AAV2; SEQ ID NO: 49)
-YYLSRTNTPSGTTTQSRLQFSQAGASDIRDQSRNWLPGP;
preferably SEQ ID NO: 41 (HVR5 of #667).

In preferred embodiments, the HVR7 sequence from the donor AAV capsid consists of:

SX₁X₂X₃WTX₄ATKYH,

wherein X₁ is D, E or N, preferably N; X₂ is Y or F, preferably F; X₃ is S or P, preferably P; X₄ is G or A, preferably A.

In more preferred embodiments, the HVR7 sequence from the donor AAV capsid is selected from the group consisting of: SEYSWTGATKYH (HVR7 of AAV2 and #1010; SEQ ID NO: 50); SNFPWTGATKYH (HVR7 of AAV13; SEQ ID NO: 51); SNFPWTAATKYH (HVR7 of #704; SEQ ID NO: 52); SEYSWTAATKYH (HVR7 of #767; SEQ ID NO: 53); SNYSWTGATKYH (HVR7 of #508; SEQ ID NO: 54); SEFPWTAATKYH (HVR7 of #2320; SEQ ID NO: 55); preferably SEQ ID NO: 52 (HVR7 of #704).

In preferred embodiments, the HVR10 sequence from the donor AAV capsid consists of:

TEQYGX₁VSX₂NLQX₃X₄NX₅X₆X₇X₈TX₉X₁₀VNX₁₁QGX₁₂LX₁₃GMVWQNRD,

wherein X₁ is S, T, A, Y or N, preferably Y; X₂ is T or N, preferably N; X₃ is R, N or S, preferably N; X₄ is G or S, preferably S; X₅ is R, A or T, preferably T; X₆ is Q, G, R or A, preferably G; X₇ is A or P, preferably P; X₈ is A, T or S, preferably T; X₉ is A, S, G or E, preferably G; X₁₀ is T, D or N, preferably T; X₁₁ is T, A, H or N, preferably H; X₁₂ is A, I or V, preferably A; and X₁₃ is P or S, preferably P.

In more preferred embodiments, the HVR10 sequence from the donor AAV capsid is selected from the group consisting of:

(HVR10 of AAV2 and #129; SEQ ID NO: 56)
-TEQYGSVSTNLQRGNRQAATADVNTQGVLPGMVWQDRD;
(HVR10 of AAV13; SEQ ID NO: 57)
-TEQYGTVSNNLQNSNAGPTTGTVNHQGALPGMVWQDRD;
(HVR10 of #704; SEQ ID NO: 58)
-TEQYGYVSNNLQNSNTGPTTGTVNHQGALPGMVWQDRD;
(HVR10 of #2731; SEQ ID NO: 59)
-TEQYGSVSTNLQRGNRRAATADVNTQGVLPGMVWQDRD;
(HVR10 of #1010; SEQ ID NO: 60)
-TEQYGYVSNNLQNSNRQAATADVNTQGVLPGMVWQDRD;
(HVR10 of #508; SEQ ID NO: 61)
-TEQYGYVSNNLQNSNTAATTETVNHOGALPGMVWQDRD;
(HVR10 of #1350; SEQ ID NO: 62)
-TEQYGYVSNNLQSGNTQAATGTVNHQGALPGMVWQDRD;
(HVR10 of #3142; SEQ ID NO: 63)
TEQYGNVSNNLQNSNTGPTTENVNNQGALPGMVWQDRD;
and
(HVR10 of #M258; SEQ ID NO: 64)
-TEQYGYVSNNLQNSNTAPSTGTVNHQGALPGMVWQDRD;
preferably SEQ ID NO: 58 (HVR10 of #704).

In preferred embodiments, the recombinant hybrid AAV capsid protein comprises or consists of a sequence selected from the group consisting of the sequences SEQ ID NO:68, 71, 74, 77, 80, 83, 86, 89, 92, 98, 104 and the sequences having at least 85%, 90%, 95%, 97%, 98% or 99% identity with said sequences; more preferably wherein the amino acid sequence variant has no mutations in at least the HVR sequences from the donor AAV capsid protein or all the HVR sequences.

In preferred embodiments, the hybrid AAV capsid protein has a seroprevalence equivalent to the seroprevalence of the acceptor AAV capsid protein. As used herein “seroprevalence” refers to the human seroprevalence, which means the level of anti-AAV antibodies binding to an AAV capsid serotype present in a human population and expressed as seric antibodies or immunoglobulins. The seroprevalence of an AAV capsid is measured using a cohort of human sera and standard assays that are well known in the art and disclosed for example in Meliani et al., Hum Gene Ther Methods. 2015 April; 26(2): 45-53. doi: 10.1089/hgtb.2015.037 and WO 2022/003211.

### Transgene Expression Cassette Driven by Hepatocyte-Specific Promoter

The hybrid AAV vector according to the invention comprises a transgene of interest operably linked to an hepatocyte-specific promoter.

As used herein, an “hepatocyte-specific promoter” refers to a natural or synthetic promoter able to drive the expression of a gene specifically in hepatocytes and derived liver tissue or organ. “hepatocyte-specific expression” refers to the preferential or predominant expression of a transgene in hepatocytes and derived liver tissue or organ as compared to other cell(s), tissues or organs. The gene under control of the hepatocyte-specific promoter has high expression levels in hepatocytes, and derived liver tissue or organ as compared to low expression levels in other cells, tissues or organs. In particular, “liver-specific expression” refers to the preferential or predominant expression of a transgene in vivo, in liver tissue or organ of an individual administered with a rAAV vector particle according to the invention, as compared to other tissues or organs of the individual. According to particular embodiments, at least 50% of the transgene occurs within the liver; preferably at least 60%, at least 70%, at least 75%, at least 80%, at least 85%, at least 90%, at least 95%, at least 97%, at least 99% of the transgene expression occurs in the liver. Transgene expression level in the liver may be measured by standard assays that are well-known in the art and disclosed in the present application.

As used herein, the term “individual” or “patient” denotes a mammal. Preferably, a patient or individual according to the invention is a human.

The transgene is operably linked to the hepatocyte-specific promoter for expression of the transgene in hepatocytes and derived liver tissue or organ. The term “operably linked” as used herein refers to the arrangement of various nucleic acid elements such that the elements are functionally connected and are able to interact with each other. The transgene expression cassette is a nucleic acid construct comprising the transgene operably linked to the hepatocyte-specific promoter. The rAAV viral vector comprises the transgene in a form expressible in liver cells, tissue or organ.

Various hepatocyte-specific promoters that can be used in liver-directed rAAV vectors are well-known in the art and easily available, for example from the Liver Specific Gene Promoter Database compiled the Cold Spring Harbor Laboratory or other databases.

In some embodiments, the hepatocyte-specific promoter is a promoter derived from any one of the following genes: Alpha-1-Antitrypsin (SERPINA1 or AAT), Albumin (ALB), Thyroxine-Binding Globulin (SERPINA7 or TBG), Transthyretin (TTR), Apolipoprotein A1 (APOA1), Complement Factor B (CFB), Ketohexokinase (KHK), Hemopexin (HPX), Nicotinamide N-methyltransferase (NNMT), (liver) Carboxylesterase 1 (CES1), Protein C (PROC), Apolipoprotein C3 (APOC3), mannan-binding lectin serine protease 2 (MASP2), Hepcidin antimicrobial peptide (HAMP), Serpin peptidase inhibitor, clade C (antithrombin), member 1 (SERPINC1), Fibrinogen Beta (FGB), Transferrin (TF), Insulin-Like Growth Factor II (IGF2) (promoter P1), and Alpha-Fetoprotein (AFP).

In some embodiments, the promoter is derived from a hepatitis virus, in particular human hepatitis virus such as HBV. A particular example of hepatitis virus promoter is HBV core promoter (CP HBV; Quarleri J., World J Gastroenterol., 2014, 20, 425-435. (doi: 10.3748/wjg.v20.i2.425).

In some particular embodiments, the hepatocyte-specific promoter is selected from the group comprising: an alpha-1 antitrypsin (SERPINA1 or AAT) promoter, a transthyretin (TTR) promoter, an albumin (ALB) promoter, a thyroxine-binding globulin (TBG) promoter and an Hepatitis B virus (HBV) core promoter.

The promoter may be a full-length promoter, a minimal promoter, or a modified promoter derived from any one of the genes disclosed herein. In particular embodiments, the promoter is a minimal-promoter derived from any one of the genes disclosed herein. In particular embodiments, the promoter is a human promoter derived from any one of the genes disclosed herein, preferably a human minimal-promoter derived from any one of the genes disclosed herein. In particular embodiments, the promoter is a modified promoter derived from any one of the genes disclosed herein.

A particular example of human AAT promoter comprises the sequence SEQ ID NO: 107. Particular examples of Thyroxine-binding globulin (TBG) promoters include TBG promoter and TBG2 promoter corresponding to the fragment of −435 bp to −26 bp from transcription start site (TSS) in the TBG promoter region (Zhongai et al., Gene, 2012, 506, 289-294).

The hepatocyte-specific promoter may be a constitutive or inducible promoter, preferably a constitutive promoter.

In particular embodiments, the hepatocyte-specific promoter is associated to an enhancer sequence capable of enhancing liver-specific expression of genes. The hepatocyte-specific enhancer may be derived from cis-regulatory modules (CRMs) containing clusters of evolutionary conserved transcription factor binding site motifs (TFBS) associated with robust-hepatocyte specific expression. Non-limiting examples of TFBS able to enhance liver-specific expression of genes include the binding sites for hepatocyte nuclear factor HNF1, HNF3, HNF4, HNF6, in particular HNF1; C/EBP; LEF1; FOX; IRF; LEF1/TCF; Tal1β/E47; MyoD and combinations thereof. Cis-regulatory modules (CRMs) and derived enhancers such as artificial enhancers useful in the practice of the present invention include those described in Chuah et al., Mol Ther. 2014, 22, 1605-13; Nair et al., Blood, 2014 123, 20, 3195-9 and WO 2009/130208. Other hepatocyte-specific enhancers useful in the practice of the present invention may be obtained by rearranging the transcription factor binding sites (TFBS) that are present in known enhancer sequences such as those disclosed in Chuah et al . . . , Nair et al., precited and WO2009130208. Said rearrangement may encompass changing the order of the TFBSs and/or changing the position of one or more TFBS relative to the other TFBS and/or changing the copy number of one or more of the TFBS. In particular embodiments, the enhancer comprises at least two, such as 2, 3, 4, or more copies of one or more of the TFBS recited before. Further examples of enhancers which can be used in the present invention include: the distal albumin regulatory region, enhancer II (EII) of the human hepatitis B virus; apolipoprotein E (ApoE control region), in particular the human ApoE control region (or Human apolipoprotein E/C-I gene locus, hepatic control region HCR-1; Genbank accession number U32510, SEQ ID NO:108); TTR enhancer (Samadani et al., Gene Expr., 1996, 6, 23-33); and HS-CR8 enhancer (Chuah et al., Mol Ther. 2014, 22, 1605-1613. doi: 10.1038/mt.2014.114).

Various hybrid or chimeric promoters that can be used in liver-directed rAAV vectors are well-known in the art. The hepatocyte-specific chimeric promoter designated HS-CRM8-TTR comprises a minimal transthyretin promoter (TTT) combined with the Hepatocyte-specific Cis-regulatory modules HNF1, FOX, C/EBP, LEF1, LEF1/TCF and MyoD (Chuah et al., Mol. Ther., 2014, 22, 1605-1613). The Hybrid hepatocyte-specific promoter (HLP) is a 251-bp fragment containing a 34-bp core enhancer from the human apolipoprotein hepatic control region, upstream of a modified 217-bp SERPINA1 gene promoter in which the distal X and the proximal A+B regulatory domains were brought together (McIntosh et al., Blood, 2013, 121, 3335-3344). The hybrid hepatocyte-specific promoter (LSP) comprises of two copies of Alpha 1 Microglobulin/Bikunin enhancer coupled to the core promoter of human Thyroxine-Binding Globulin (TBG), and a leader sequence (Charles R. et al., Blood Coag. Fibrinol, 1997, 8: S23-S30). Expression is further stabilized by the inclusion of a woodchuck hepatitis virus post-transcriptional regulatory element (WPRE) (Wang et al., PNAS, 1999, 96, 3906-3910). The hybrid hepatocyte-specific promoter, designated ET is composed of randomly assembled hepatocyte-specific transcription factor binding sites linked to the murine transthyretin promoter (Vigna et al., Mol. Ther., 2005, 11, 763-775). The hybrid hepatocyte-specific promoter, designated HCB (Hepatic Combinatorial Bundle) is composed of the HNF1 hepatocyte nuclear factor binding site, a-microglobulin/bikunin precursor shortened sequence (AbpShort), a 41-bp fragment from the Xenopus laevis albumin 5′ UTR containing the HP1 hepatocyte-specific transcription factor binding site as well as a TATA box (SynO) and a transcription start site (TSS), (Brown et al., Molecular Therapy: Methods & Clinical Development, 2018, 9, 57-69). The LP1 promoter consists of core liver-specific elements from HCR (base pairs 134 to 442 of GenBank accession number HSU32510) and hAAT (base pairs 1747 to 2001 of GenBank accession number K02212; Nathwani et al., Blood, 2006, 107, 2653-2661); LP1 promoter sequence corresponds to SEQ ID NO: 117. The HSE promoter is an artificial promoter consisting of multimerized heat shock elements (HSE; GTCGAGAACGTTCTAGAACGTCG (SEQ ID NO:12) from heat shock protein (HSP) genes in combination with a TATA box (Ortner et al., Cell Stress Chaperones, 2015, 20, 277-288).

A synthetic hepatocyte-specific promoter may be a modified, chimeric or hybrid promoter derived from any of the genes disclosed herein, or a combination thereof. Synthetic hepatocyte-specific promoters are disclosed in WO 2020/104424, in particular in Tables 1 to 3; SERPINA1_COMP_D is a synthetic promoter derived from the alpha-1-antitrypsin gene, corresponding to the sequence SEQ ID NO: 29 in WO 2020/104424 or SEQ ID NO: 121 in the present disclosure.

Other particular examples of synthetic hepatocyte-specific promoters are disclosed in the examples: TBG+TSS: synthetic TBG promoter (SEQ ID NO: 118); TTR: synthetic transthyretin promoter (TTR) comprising TTR enhancer (Samadani et al., Gene Expr., 1996, 6, 23-33) and human TTR promoter sequences (SEQ ID NO: 119); HSE: synthetic promoter comprising HS-CR8 enhancer (Chuah et al., Mol Ther. 2014, 22, 1605-1613. doi: 10.1038/mt.2014.114) and human TTR promoter sequences (SEQ ID NO: 120).

Preferred examples of synthetic hepatocyte-specific promoters include: LP1, HLP, HSE, TBG+TSS and SERPINA1_COMP_D.

In some particular embodiments, the hepatocyte-specific promoter is selected from the group comprising: alpha-1 antitrypsin (SERPINA1 or AAT) promoter, transthyretin (TTR) promoter, albumin (ALB) promoter, thyroxine-binding globulin (TBG) promoter, Hepatitis B virus (HBV) core promoter, and modified, hybrid and/or chimeric promoters derived thereof. AAT promoter is preferably hAAT (SEQ ID NO: 107), AAT-derived promoter LP1 (SEQ ID NO: 117), AAT-derived promoter HLP or AAT-derived promoter SERPINA1_COMP_D (SEQ ID NO: 121). TTR promoter is preferably SEQ ID NO: 119 or TTR-derived promoter HSE (SEQ ID NO: 120). TBG promoter includes full-length TBG promoter, minimal TBG promoter (TBG2), and TBG-derived promoter TBG+TSS (SEQ ID NO: 118); preferably TBG2 or TBG+TSS.

In some particular embodiments, an enhancer sequence such as the ApoE control region, preferably human ApoE control region, is associated to an hepatocyte-specific promoter such as those listed above, and in particular such as the hAAT promoter.

In some embodiments, the transgene is operably linked to further regulatory sequences capable of further controlling transgene expression in liver cells or tissue, such as without limitation, terminator, intron, tissue-specific silencer and post-transcriptional regulatory element. Therefore, the transgene, promoter or promoter/enhancer and further regulatory sequences are included in a nucleic acid construct forming the transgene expression cassette.

In particular embodiments, the transgene expression cassette comprises an intron, in particular an intron placed between the promoter and the transgene. An intron is introduced to increase mRNA stability and protein production. In addition, a modified intron designed to decrease the number of, or even totally remove, alternative open reading frames (ARFs) found in said intron can significantly improve the expression of the transgene. Furthermore, by decreasing the number of ARFs within the intron included within the construct of the invention, it is believed that the construct immunogenicity is also decreased. Preferably, ARFs are removed whose length spans over 50 bp and have a stop codon in frame with a start codon. ARFs may be removed by way of nucleotide substitution, insertion or deletion, preferably by nucleotide substitution. For example, an ATG or a GTG may be replaced by a CTG, which is not a start codon, within the sequence of the intron of interest. Examples of introns which can be used in the present invention include those disclosed in WO 2020/212626, in particular human beta globin b2 (or HBB2; SEQ ID NO: 109) intron, modified HBB2 intron (SEQ ID NO: 110); a coagulation factor IX (FIX) intron, in particular hFIX, in particular derived from first intron (SEQ ID NO: 111), and modified intron thereof (SEQ ID NO: 112); chicken beta-globin intron (SEQ ID NO:113) and modified intron thereof (SEQ ID NO: 114); and SV40 intron. Preferred introns are hFIX intron and HBB2 intron, including modified introns thereof (SEQ ID NO: 109 à 112).

In particular embodiments, the transgene expression cassette further comprises a silencer, in particular a tissue-specific silencer able to suppress transgene expression in cell(s) or tissue other than liver cell(s) or tissue. Examples of tissue-specific silencer include miRNAs. The inclusion of microRNA (miRNA) target sequences in the vector expression cassette can help to eliminate off-target transgene expression from transduced cells that express the corresponding miRNA. Moreover transgene expression levels can be improved by the inclusion, in the transgene expression cassette, of post-transcriptional regulatory elements such as the Woodchuck hepatitis virus (WHV) post-transcriptional regulatory element (WPRE), able to increase transcript levels and/or stability.

In particular embodiments, the transgene expression cassette further comprises a transcription termination signal (polyadenylation signal) operably linked to the transgene coding sequence (i.e., at the 3′-end of the coding sequence). Examples of polyA which can be used in the present invention include the bovine growth hormone polyadenylation signal (BGHpA), the human beta globin b2 (HBB2) polyadenylation signal, and the Simian virus 40 polyadenylation signal (SV40pA). In preferred embodiments, the nucleic acid construct further comprises a BGH polyA, preferably comprising the sequence SEQ ID NO: 115.

In some particular embodiments, the transgene expression cassette comprises, in the 5′ to 3′ orientation, an hepatocyte-specific promoter; the transgene coding sequence; and a polyadenylation signal such as BGH or HBBpolyA. In some preferred embodiments, the expression cassette further comprises one or more further regulatory elements chosen from an enhancer, preferably human ApoE control region and an intron such as hFIX or HBB2 intron. In some more particular embodiments, the hepatocyte-specific promoter is selected from the group comprising: alpha-1 antitrypsin (SERPINA1 or AAT) promoter, in particular hAAT promoter (SEQ ID NO: 107), and AAT-derived promoters LP1 (SEQ ID NO: 117), HLP or SERPINA1_COMP_D (SEQ ID NO: 121); transthyretin (TTR promoter), in particular TTR promoter comprising SEQ ID NO: 119 and TTR-derived promoter HSE (SEQ ID NO: 120); thyroxine-binding globulin (TBG) promoter including full-length TBG promoter, minimal TBG promoter (TBG2), and TBG-derived promoter TBG+TSS (SEQ ID NO: 118); preferably TBG2 or TBG+TSS; albumin (ALB) promoter, and Hepatitis B virus (HBV) core promoter.

By “transgene of interest” or “gene of interest”, it is meant a gene useful for a particular application, such as with no limitation, diagnosis, reporting, modifying, therapy and genome editing. For example, the gene of interest may be a therapeutic gene, a reporter gene or a genome-editing enzyme.

By “gene of interest for therapy”, “gene of therapeutic interest”, or “heterologous gene of interest”, it is meant a therapeutic gene or a gene encoding a therapeutic protein, peptide or RNA. In particular, the gene of interest for therapy is any transgene of interest for liver-targeted gene therapy.

The gene of interest is any nucleic acid sequence capable of modifying a target gene or target cellular pathway, in liver cells, tissue or organ. For example, the gene may modify the expression, sequence or regulation of the target gene or cellular pathway. In some embodiments, the gene of interest is a functional version of a gene or a fragment thereof. The functional version of said gene includes the wild-type gene, a variant gene such as variants belonging to the same family and others, or a truncated version, which preserves the functionality of the encoded protein at least partially. A functional version of a gene is useful for replacement or additive gene therapy to replace a gene, which is deficient or non-functional in a patient. In other embodiments, the gene of interest is a gene which inactivates a dominant allele causing an autosomal dominant genetic disease. A fragment of a gene is useful as recombination template for use in combination with a genome editing enzyme.

Alternatively, the gene of interest may encode a protein of interest for a particular application, (for example an antibody or antibody fragment, a genome-editing enzyme) or a RNA. In some embodiments, the protein is a therapeutic protein including a therapeutic antibody or antibody fragment, or a genome-editing enzyme. In some embodiments, the RNA is a therapeutic RNA.

The gene of interest is a functional gene able to produce the encoded protein, peptide or RNA in liver cells, tissue or organ. In some embodiments, the gene of interest is a human gene. In some embodiments, the sequence of the gene of interest is optimized for expression in the treated individual, preferably a human individual. Sequence optimization may include a number of changes in a nucleic acid sequence, including codon optimization, increase of GC content, decrease of the number of CpG islands, decrease of the number of alternative open reading frames (ARFs) and/or decrease of the number of splice donor and splice acceptor sites. Sequence optimization may also include reduction of sequence length. The transgene may comprise a shortened sequence to facilitate transgene cloning in rAAV vector or improve transgene expression in liver cells or tissue.

The transgene may encode a protein that remains in the liver after synthesis. Alternatively, the transgene may encode a protein that is secreted in the bloodstream after synthesis. The liver is an attractive organ for gene therapy to treat serum protein deficiencies or to produce therapeutic proteins in the serum. The liver advantageously performs the proper posttranslational modifications necessary for full activity for many serum proteins, such as the y-carboxylation of coagulation factors, and the rich blood supply of the liver bathing the transduced hepatocytes facilitates the secretion of protein products into the bloodstream.

To express proteins that are secreted in the bloodstream, the transgene advantageously comprises a signal peptide or signal sequence at the 5′end of the coding sequence. Signal peptides (SP) are short peptide sequences which are present at the N-terminus of secretory proteins and are used to target proteins for secretion. Multiple signal peptides are known in the art and publically available (see in particular, Signal Peptide Website and SPdb sequence databases; Puzzo et al., Sci. Transl. Med., 2017, 9 (418): doi: 10.1126). In addition, methods to select suitable SP sequences for efficient protein secretion are known in the art (see in particular, Stern et al., BMC Proc., 2011, 5 (suppl 8): 013). The signal peptide may be the endogenous or native signal peptide or a heterologous signal peptide. Heterologous signal peptides useful in the present invention include thoses disclosed in WO 2020/212626, such as in particular: alpha-1 antitrypsin, synthetic mut1, synthetic mut3, chymotrypsinogen B2 (CTRB2; positions 1 to 18 of Uniprot accession number Q6GPI1 or NCBI accession number NP_001020371) and plasma protease inhibitor C1 (positions 1 to 22 of Uniprot accession number P05155).

Proteins that are secreted in the bloodstream may be expressed in the form of fusion proteins, wherein the protein of interest is linked to a protein stabilizing moiety. The protein stabilizing moiety is any protein moiety which increases the half-life or duration of action of the therapeutic protein/peptide that is attached to it and is suitable for therapeutic application. Various protein stabilizing moieties that have been used to stabilize therapeutic proteins are known in the art (see for example Sven Berger, Peter Lowe & Michael Tesar (2015) Fusion protein technologies for biopharmaceuticals: Applications and challenge, mAbs, 7:3, 456-460, DOI: 10.1080/19420862.2015.1019788). Examples of protein stabilizing moieties which can be used in the present invention include without limitation: serum transport proteins such as albumin, alpha-fetoprotein (AFP; Beattie and Dugaiczyk, Gene 1982, 20, 415-422), afamin (AFM; Lichenstein et al., J. Biol. Chem., 1994, 269, 18149-18154) and vitamin D binding protein (DBP; Cooke and David, J. Clin. Invest., 1985, 76, 2420-2424), in particular human serum albumin; immunoglobulin Fc fragment; human chorionic gonadotropin carboxy-terminal peptide (CTP); Receptor (fused to its ligand (GHR fused to GH); latency-associated peptide of TGF-beta (linked to a cleavage site for metalloprotease); and functional fragments and variant of the cited proteins.

Proteins that are secreted in the bloodstream may be expressed in the form of fusion proteins, wherein the protein of interest is linked to a target cell receptor binding, to target the secreted protein from the bloodstream to a target organ.

The RNA is advantageously complementary to a target DNA or RNA sequence or binds to a target protein. For example, the RNA is an interfering RNA such as a shRNA, a microRNA, a guide RNA (gRNA) for use in combination with a Cas enzyme or similar enzyme for genome editing, an antisense RNA capable of exon skipping such as a modified small nuclear RNA (snRNA) or a long non-coding RNA. The interfering RNA or microRNA may be used to regulate the expression of a target gene having altered expression in the liver. The guide RNA in complex with a Cas enzyme or similar enzyme for genome editing may be used to modify the sequence of a target gene, in particular to correct the sequence of a mutated/deficient gene or to modify the expression of a target gene having altered expression in the liver. The antisense RNA capable of exon skipping is used in particular to correct a reading frame and restore expression of a deficient gene having a disrupted reading frame. In some embodiments, the RNA is a therapeutic RNA.

The genome-editing enzyme according to the invention is any enzyme or enzyme complex capable of modifying a target gene or target cellular pathway in liver cells. For example, the genome-editing enzyme may modify the expression, sequence or regulation of the target gene or cellular pathway. The genome-editing enzyme is advantageously an engineered nuclease, such as with no limitations, a meganuclease, zinc finger nuclease (ZFN), transcription activator-like effector-based nuclease (TALENs), Cas enzyme from clustered regularly interspaced palindromic repeats (CRISPR)-Cas system and similar enzymes. The genome-editing enzyme, in particular an engineered nuclease such as Cas enzyme and similar enzymes, may be a functional nuclease which generates a double-strand break (DSB) or single-stranded DNA break (nickase such as Cas9 (D10A) in the target genomic locus and is used for site-specific genome editing applications, including with no limitations: gene correction, gene replacement, gene knock-in, gene knock-out, mutagenesis, chromosome translocation, chromosome deletion, and the like. For site-specific genome editing applications, the genome-editing enzyme, in particular an engineered nuclease such as Cas enzyme and similar enzymes may be used in combination with a homologous recombination (HR) matrix or template (also named DNA donor template) which modifies the target genomic locus by double-strand break (DSB)-induced homologous recombination. In particular, the HR template may introduce a transgene of interest into the target genomic locus or repair a mutation in the target genomic locus, preferably in an abnormal or deficient gene having altered expression in the liver. Alternatively, the genome-editing enzyme, such as Cas enzyme and similar enzymes may be engineered to become nuclease-deficient and used as DNA-binding protein for various genome engineering applications in liver cells, tissue or organ, such as with no limitation: transcriptional activation, transcriptional repression, epigenome modification, genome imaging, DNA or RNA pull-down and the like.

In particular embodiments, the transgene encodes a protein with a therapeutic effect on hepatocytes or a therapeutic protein that is secreted from the liver into the bloodstream; preferably selected from the group consisting of: Acid Alpha-Glucosidase (GAA), FGF23 antagonist (FGF-23 C-ter fragment), Amylo-Alpha-1,6-Glucosidase, 4-Alpha-Glucanotransferase (AGL), Glucose-6-phosphate dehydrogenase (G6PD) and Uridine Diphosphate Glucuronosyltransferase isoform 1A1 (UGT1A1).

The rAAV vector particle according to the invention comprises the hybrid AAV capsid protein and the rAAV vector genome comprising the transgene expression cassette flanked by ITRs. The rAAV vector particle according to the invention is suitable for liver-directed gene therapy. The genome of the rAAV vector may either be a single-stranded or self-complementary double-stranded genome (McCarty et al, Gene Therapy, 2003 December, 10(26), 2112-2118). Self-complementary vectors are generated by deleting the terminal resolution site (trs) from one of the AAV terminal repeats. These modified vectors, whose replicating genome is half the length of the wild-type AAV genome have the tendency to package DNA dimers. In particular embodiments, the AAV vector is a pseudotyped vector, i.e. its genome and capsid are derived from AAVs of different serotypes. In preferred embodiments, the genome of the pseudotyped vector is derived from AAV2. The rAAV vector particle may be obtained using standard AAV production methods that are well-known in the art (Review in Aponte-Ubillus et al., Applied Microbiology and Biotechnology, 2018, 102:1045-1054). AAV vectors are usually produced by co-transfecting cells suitable for AAV production with a plasmid containing recombinant AAV vector genome comprising the gene of interest inserted in an expression cassette, flanked by AAV ITRs (AAV transfer plasmid), and plasmid(s) expressing AAV Rep and Cap proteins. Alternatively, producer cells which stably express AAV Rep and Cap proteins may be transfected with an AAV transfer plasmid. Briefly, following transfection with above plasmid(s) in the presence of sufficient helper function to permit packaging of the rAAV vector genome into AAV capsid particle, the cells are incubated for a time sufficient to allow the production of AAV vector particles, the cells are then harvested, lysed, and AAV vector particles are purified by standard purification methods such as affinity chromatography and Iodixanol or Cesium Chloride density gradient ultracentrifugation.

Another aspect of the invention relates to a method of preparation of a hybrid AAV vector particle that enhances transgene expression in the liver, comprising the steps of:
a) providing at least two recombinant AAV capsid proteins from different AAV serotypes, an acceptor AAV capsid protein and at least one donor AAV capsid protein;
b) replacing at least one hypervariable region (HVR) selected among the HVR1, HVR5, HVR7 or HVR10 sequence of the acceptor AAV capsid protein with a different HVR sequence from the corresponding HVR of a donor AAV capsid protein, to obtain a recombinant hybrid AAV capsid protein;
c) providing a first nucleic acid construct comprising a sequence coding for the recombinant hybrid AAV capsid protein in expressible form;
d) providing a second nucleic acid construct comprising a transgene expression cassette driven by an hepatocyte-specific promoter and flanked by AAV ITRs;
e) obtaining at least one plasmid comprising the first and second nucleic acid constructs, the AAV Rep gene and appropriate adenoviral sequences providing help for AAV production;
f) introducing the at least one plasmid in AAV producer cell line for a time sufficient for production of hybrid AAV vector particles comprising the recombinant hybrid AAV capsid protein and the transgene expression cassette, which enhance transgene expression in the liver.

The invention also relates to an isolated hepatocyte, in particular an hepatocyte from an individual, which is genetically modified or transformed with a vector of the invention. The individual is advantageously a patient to be treated.

### Pharmaceutical Compositions and Therapeutic Uses

Another aspect of the invention is a pharmaceutical composition comprising at least an active agent selected from an AAV vector particle or a cell (hepatocyte) of the invention, and a pharmaceutically acceptable carrier.

The AAV vector particle and derived hepatocyte or pharmaceutical composition of the invention may be used for treating diseases by gene therapy, in particular targeted gene therapy directed to hepatocytes and derived liver, tissue or organ. The hepatocyte and derived pharmaceutical composition of the invention may be used for treating diseases by cell therapy, in particular cell therapy directed to liver (i.e., liver-directed cell therapy).

As used herein “Gene therapy” refers to a treatment of an individual which involves delivery of nucleic acid of interest into an individual's cells for the purpose of treating a disease. Delivery of the nucleic acid is generally achieved using a delivery vehicle, also known as a vector. The rAAV vector particle of the invention may be employed to deliver a gene to a patient's cells.

As used herein “Cell therapy” refers to a process wherein cells stably transduced by a rAAV vector particle of the invention are delivered to the individual in need thereof by any appropriate mean such as for example by intravenous injection (infusion), or injection in the tissue of interest (implantation or transplantation). In particular embodiments, cell therapy comprises collecting cells from the individual, modifying the individual's cells with the rAAV vector particle of the invention, and administering the stably transduced cells back to the patient. As used herein “cell” refers to isolated cell, natural or artificial cellular aggregate, bioartificial cellular scaffold and bioartificial organ or tissue.

Gene therapy can be performed by gene transfer, gene editing, exon skipping, RNA-interference, trans-splicing or any other genetic modification of any coding or regulatory sequences in the cell, including those included in the nucleus, mitochondria or as commensal nucleic acid such as with no limitation viral sequences contained in cells.

The two main types of gene therapy are the following:
a therapy aiming to provide a functional replacement gene for a deficient/abnormal gene: this is replacement or additive gene therapy;
a therapy aiming at gene or genome editing: in such a case, the purpose is to provide to a cell the necessary tools to correct the sequence or modify the expression or regulation of a deficient/abnormal gene so that a functional gene is expressed or an abnormal gene is suppressed (inactivated): this is gene editing therapy.

In additive gene therapy, the gene of interest may be a functional version of a gene, which is deficient or mutated in a patient, as is the case for example in a genetic disease. In such a case, the gene of interest will restore the expression of a functional gene. Thus, by gene editing or gene replacement a correct version of this gene is provided in target cells, in particular hepatocytes of affected patients, this may contribute to effective therapies against the disease.

Gene or genome editing uses one or more gene(s) of interest, such as:
a gene encoding a therapeutic RNA as defined above such as an interfering RNA like a shRNA or a microRNA, a guide RNA (gRNA) for use in combination with a Cas enzyme or similar enzyme, or an antisense RNA capable of exon skipping such as a modified small nuclear RNA (snRNA); and
a gene encoding a genome-editing enzyme as defined above such as an engineered nuclease like a meganuclease, zinc finger nuclease (ZFN), transcription activator-like effector-based nuclease (TALENs), Cas enzyme or similar enzymes; or a combination of such genes, and maybe also a fragment of a functional version of a gene for use as recombination template, as defined above.

Liver-targeted gene therapy may be used to express a functional gene in the liver to replace a needed protein, to block the expression of an altered or undesired gene product, or to restore hepatocyte function in a degenerating liver.

Liver-targeted gene therapy may also be used to express a therapeutic protein that is secreted from the liver. The therapeutic protein, including a peptide or antibody is secreted from the liver cells into the blood stream where it can be delivered to other target tissues.

Liver-targeted gene therapy may be used to treat a disease associated with altered gene expression in the liver or altered liver function, or to express a therapeutic protein that is secreted from the liver. The disease may be an acquired, complex or genetic disease. The altered gene expression in the liver may cause a disease in the liver (hepatic disease sensu stricto) or elsewhere in the body. The disease may affect the structure or function of the liver or other target tissue(s). The disease may be caused by trauma, infection, degeneration, structural or metabolic defects, tumors, autoimmune disorders, stroke or others.

Non-limiting examples of diseases that can be treated by liver-targeted gene therapy include genetic disorders, cancer, degenerative diseases, auto-immune diseases and infectious diseases affecting the liver or other tissues or organs. Infectious diseases include in particular viral hepatitis such as hepatitis B and hepatitis C. Cancers include in particular hepatomas, cholangiocarcinomas, liver metastatic tumors and extrahepatic tumors. Liver degenerative diseases result in particular from pharmaceutical treatment, drug or alcohol abuse. Liver degenerative diseases include liver cirrhosis.

In particular embodiments, liver-targeted gene therapy may be used for treating monogenic disorders due to defect in gene expressed in liver cells, liver viral infections, liver cancers, or multifactorial disorders treated by targeting a single gene.

In some preferred embodiments, liver-targeted gene therapy is used for treating genetic diseases associated with altered gene expression in the liver. In other preferred embodiments, liver-targeted gene therapy is used for treating other genetic diseases by expressing the therapeutic gene in the liver; the therapeutic gene encodes preferably a protein which is secreted from the liver cells into the blood stream where it can be delivered to other target tissues.

Non-limiting examples of genetic diseases that can be treated by liver-targeted gene therapy include:
Hemophilia A (Factor VIII; F8 gene), Hemophilia B (Factor IX; F9 gene), and other coagulation factors deficiencies such as von Willebrand factor (VWF gene), Factor X (F10 gene), Factor VII (F7 gene), and others;
-Protein C deficiency: Protein C, Inactivator Of Coagulation Factors Va And VIIIa (PROC gene);
-Primary hyperoxaluria type 1 (PH1): Alanine-Glyoxylate Aminotransferase (AGXT or AGT);
Crigler-Najjar syndrome (Hyperbilirubinemia): Uridine Diphosphate Glucuronosyltransferase isoform 1A1 (UGT1A1) gene encoding the enzyme that conjugates bilirubin;
Familial Hypercholesterolemia (FH): LDLR, APOB, PCSK9, VLDLR genes and other lipid metabolic disorders;
Maple syrup urine disease (MSUD); Branched-chain alpha-keto acid dehydrogenase complex (BCKDHA, BCKDHB, DBT, DLD) genes;
Progressive familial intrahepatic cholestasis type 3 (PFIC3): ATP Binding Cassette Subfamily B Member 4 (ABCB4) gene;
Phenylketonuria (PKU): Phenylalanine hydroxylase (PAH) gene;
Tyrosinemia: Fumarylacetoacetate Hydrolase (FAH) gene;
Lysosomal storage diseases, such as Glycogen storage diseases (GSD), in particular Type I (GSDIA: Glucose-6-Phosphatase deficiency (G6PC1 gene); GSDIB: Glucose-6-Phosphatase translocase deficiency (SLC37A4 gene), Type II (Pompe disease; Acid Alpha-Glucosidase (GAA) gene), Type III (Cori's disease or Forbes' disease; Amylo-Alpha-1,6-Glucosidase, 4-Alpha-Glucanotransferase (AGL) gene), Type IV (Anderson's disease; 1,4-Alpha-Glucan Branching Enzyme 1 (GBE1) gene) and Type VII (Beta-Glucuronidase (GUSB) gene); lipid storage diseases including Gaucher's and Niemann-Pick diseases (various lysosomal hydrolase genes such as Glucocerebrosidase (GBA) gene); Mucopolysaccharidoses including Type I (Hurler syndrome; Alpha-L-Iduronidase (IDUA) gene); Type II (Hunter syndrome (Iduronate-2 sulfatase (IDS) gene), Type III (N-Sulfoglucosamine Sulfohydrolase (SGHS) gene), Type VI (Maroteaux-Lamy syndrome; Arylsulfatase B (ARSB) gene); leukodystrophies such as GLD (beta-galactocerebrosidase (GALC) gene); Fabry disease (alpha-galactosidase A (GLA) gene); and others;
Ornithine transcarbamylase deficiency (OTC gene) and other urea cycle disorders;
Alpha-antitrypsin deficiency (SERPINA1 or AAT gene);
Wilson's disease: ATPase Copper Transporting Beta (ATP7B) gene;
Acute intermittent porphyria (AIP): Porphobilinogen Deaminase (HMBS) gene;
Citrullinemia type I: Argininosuccinate Synthase (AAS1) gene;
Methylmalonic acidemia (Methylmalonyl-CoA mutase (MMUT), MMAA, MMAB, MMADHC, MCEE genes);
Lipoprotein Lipase Deficiency: Lipoprotein Lipase (LPL) gene);
mitochondrial disorders such as Leber Hereditary Optic Neuropathy (LHON): ND1, ND4 and ND6 genes; and others;
Propionyl acidemia: Propionyl-coA (PCCA, PCCB) genes);
Arginine deficiency: Arginase 1 (ARGI) gene;
Transthyretrin amyloidosis (ATTR): CRISPR-Cas9 gene-editing targeting TTR gene;
G6PD deficiency: Glucose-6-phosphate dehydrogenase (G6PD gene);
Fibroblast growth factor 23 (FGF-23) related hypophosphatemic genetic diseases including: X-linked hypophosphatemia (XLH) caused by mutations in the PHEX gene; Autosomal dominant hypophosphatemic rickets (ADHR) caused by mutations in the FGF23 gene; Autosomal recessive hypophosphatemic rickets 1 (ADHR1) caused by mutations in the DMP1 gene; Autosomal recessive hypophosphatemic rickets 2 (ADHR2) caused by mutations in the ENPP1 gene; Osteoglophonic dysplasia caused by mutations in the FRFR1 gene; Jansen type metaphyseal chondrodysplasia caused by mutations in the PTH1R gene; Hypophosphatemia, dental anomalies and ectopic calcification caused by mutations in the FAM20C gene; McCune-Albright syndrome/fibrous dysplasia caused by mutations in the GNAS1 gene; Hypophosphatemia, skin and bone lesions caused by mutations in the HRAS or NRAS gene; in particular X-linked hypophosphatemia (XLH).

FGF-23 related hypophosphatemic diseases are caused by an excessive activity of FGF23. Liver-targeted gene therapy of FGF-23 related hypophosphatemic diseases such as XLH involve the expression of an FGF23 C-terminal fragment which is secreted from the liver into the bloodstream as disclosed in WO 2020/212626. The FGF23 C-terminal fragment is an antagonist of FGF23 which competes with full-length ligand for binding to the FGFR-Klotho complex and blocks FGF23 signaling.

In preferred embodiments, the disease is a genetic disease selected from the group consisting of: Hemophilia A (Factor VIII; F8 gene); Hemophilia B (Factor IX; F9 gene); X-linked hypophosphatemia (XLH); Lysosomal storage diseases such as Glycogen storage diseases, in particular GSDI (Glucose-6-Phosphatase (G6PC1) gene), GSDII (Pompe disease; Acid Alpha-Glucosidase (GAA) gene) and GSDIII (Cori's disease or Forbes' disease; Amylo-Alpha-1,6-Glucosidase, 4-Alpha-Glucanotransferase (AGL) gene); Crigler-Najjar syndrome (UGT1A1 gene) and Glucose-6-phosphate dehydrogenase (G6PD) deficiency (G6PD gene); preferably GSDI (G6PC1 gene), GSDII (GAA) gene), GSDIII (AGL gene); Crigler-Najjar syndrome (UGT1A1 gene) and Glucose-6-phosphate dehydrogenase deficiency (G6PD gene).

In the various embodiments of the present invention, the pharmaceutical composition comprises a therapeutically effective amount of rAAV vector particle or cell. In the context of the invention a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies. The term “effective dose” or “effective dosage” is defined as an amount sufficient to achieve, or at least partially achieve, the desired effect.

The effective dose is determined and adjusted depending on factors such as the composition used, the route of administration, the physical characteristics of the individual under consideration such as sex, age and weight, concurrent medication, and other factors, that those skilled in the medical arts will recognize. The effective dose can be determined by standard clinical techniques. In addition, in vivo and/or in vitro assays may optionally be employed to help predict optimal dosage ranges.

In the various embodiments of the present invention, the pharmaceutical composition comprises a pharmaceutically acceptable carrier and/or vehicle.

A “pharmaceutically acceptable carrier” refers to a vehicle in which the therapeutic is administered and that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The pharmaceutical composition may also comprise an additional therapeutic agent, in particular an agent useful for the treatment of a liver disease or a disease associated with altered gene expression in the liver according to the present disclosure.

The rAAV vector particle, cell or pharmaceutical composition of the invention may be used in combination with other biologically active agents, wherein the combined use is by simultaneous, separate or sequential administration.

The invention provides also a method for treating a disease by expression of a therapeutic gene in the liver, comprising: administering to a patient a therapeutically effective amount of the pharmaceutical composition as described above.

Another aspect of the invention relates to the rAAV vector particle, cell, pharmaceutical composition according to the present disclosure as a medicament, in particular for use in the treatment of a liver disease or a disease associated with altered gene expression in the liver according to the present disclosure.

The invention provides also a method for treating a liver disease or a disease associated with altered gene expression in the liver, comprising: administering to a patient a therapeutically effective amount of the pharmaceutical composition as described above, comprising at least an active agent selected from a rAAV vector particle or a cell of the invention, and a pharmaceutically acceptable carrier.

A further aspect of the invention relates to the use of a rAAV vector particle, cell according to the present disclosure in the manufacture of a medicament for the treatment of a liver disease or a disease associated with altered gene expression in the liver according to the present disclosure.

Another aspect of the invention relates to the use of a rAAV vector particle or a cell of the present disclosure for the treatment of a liver disease or a disease associated with altered gene expression in the liver according to the present disclosure.

A further aspect of the invention relates to a pharmaceutical composition for treatment of a liver disease or a disease associated with altered gene expression in the liver according to the present disclosure, comprising a rAAV vector particle or a cell of the present disclosure as an active component.

A further aspect of the invention relates to a pharmaceutical comprising a rAAV vector particle or a cell of the present disclosure for treating a liver disease or a disease associated with altered gene expression in the liver according to the present disclosure.

As used herein, the term “patient” or “individual” includes human and other mammalian subjects that receive either prophylactic or therapeutic treatment. Preferably, a patient or individual according to the invention is a human.

Treatment”, or “treating” as used herein, is defined as the application or administration of a therapeutic agent or combination of therapeutic agents to a patient, or application or administration of said therapeutic agents to an isolated tissue or cell line from a patient, who has a disease with the purpose to cure, heal, alleviate, relieve, alter, remedy, ameliorate, improve or affect the disease, or any symptom of the disease. In particular, the terms “treat’ or treatment” refers to reducing or alleviating at least one adverse clinical symptom associated with the disease.

The term “treatment” or “treating” is also used herein in the context of administering the therapeutic agents prophylactically.

The pharmaceutical composition of the present invention is generally administered according to known procedures, at dosages and for periods of time effective to induce a therapeutic effect in the patient. The pharmaceutical composition may be administered by any convenient route, such as in a non-limiting manner by infusion or bolus injection, by absorption through epithelial or mucocutaneous linings (e.g., oral mucosa, rectal and intestinal mucosa, etc.). The administration can be systemic, local or systemic combined with local; systemic includes parenteral and oral, and local includes local and loco-regional. Systemic administration is preferably parenteral such as subcutaneous (SC), intramuscular (IM), intravascular such as intravenous (IV) or intraarterial; intraperitoneal (IP); intradermal (ID), epidural or else. The parenteral administration is advantageously by injection or perfusion. The pharmaceutical composition of the invention may be introduced into the liver of the subject by any suitable route. In some preferred embodiments, the administration is parenteral, preferably intravascular such as intravenous (IV) or intraarterial.

The various embodiments of the present disclosure can be combined with each other and the present disclosure encompasses the various combinations of embodiments of the present disclosure.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1. Transgene expression efficacy of hybrid capsids in mouse model. FIX expression driven by hepatocyte-specific promoter (hAAT) using hybrid capsids in mice sera 1 month post-injection. Each column represents the average of activity in 3 mice expressed as μg of FIX protein per mL of sera. Standard deviations are displayed. Statistical analysis on FIX expression was performed using one-way ANOVA. Dunnett's multiple comparison test was used to compare the mean of each capsid with the mean of the AAV8 capsids. **=p<0.01.

FIG. 2. Transduction efficacy of hybrid capsids in mouse model. Vector Genome Copy Number of hybrid capsids in liver. Each column represents the average of AAV genome copy in 3 mice expressed as vector genome copy number per diploid cell. Standard deviations are displayed. Statistical analysis on VGCN was performed using one-way ANOVA. Dunnett's multiple comparison test was used to compare the mean of each capsid with the mean of the AAV8 capsids. No significant difference was observed between hybrid capsids compared to AAV8.

FIG. 3. Efficacy of hybrid capsids in mouse model. LUC expression driven by ubiquitous (CMV) promoter (in light grey, and dot) or FIX expression driven by hepatocyte-specific (hAAT) promoter (in dark grey and slanted line) using hybrid capsids in liver. Each column represents the average of transgene activity in 3 mice expressed as fold change versus

AAV8. Standard deviations are displayed. Statistical analysis on fold change was performed using one-way ANOVA. Dunnett's multiple comparison test was used to compare the mean of each capsid with the mean of the AAV8 capsids. *=p<0.05; ****=p<0.0001.

FIG. 4. Efficacy of hybrid capsids in mouse model. FIX expression using various hepatocyte-specific promoters and hybrid capsid in mice sera 1 month post-injection. Each column represents the average of activity in 3 mice expressed as μg of FIX protein per mL of sera. Standard deviations are displayed.

### EXAMPLES

### Materials and Methods

### 1. Plasmids

The plasmids containing AAV2 Rep sequence and hybrid Cap genes were disclosed previously in WO 2022/003211. A plasmid containing AAV2 Rep sequence and AAV8 Cap gene was used as control. A transgene plasmid containing a hFIX expression cassette flanked by AAV2 ITRs flanking was constructed. The transgene expression cassette comprises, in the 5′ to 3′ orientation: human ApoE control region (SEQ ID NO: 108), an hepatocyte-specific promoter, FIX intron (SEQ ID NO: 111) and a hFIX transgene construct in which human FIX cDNA is interrupted by a 1.4-kb fragment of intron 1.5, 58, 59 (SEQ ID NO: 116; George et al., Mol Ther. 2020 Sep. 2; 28 (9): 2073-2082).

The hepatocyte-specific promoters included:
hAAT: human alpha-1 antitrypsin promoter (SEQ ID NO: 107);
LP1: synthetic promoter (SEQ ID NO: 117) corresponding to SEQ ID NO: 20 in WO 2020/104424;
TBG: Thyroxine-binding globulin promoter; TBG2: fragment of-435 bp to-26 bp from transcription start site (TSS) in the TBG promoter region (TBG2); (Zhongai et al., Gene, 2012, 506, 289-294); TBG+TSS: synthetic TBG promoter (SEQ ID NO: 118);
ALB: human albumin promoter on human chromosome 4 (NCBI accession number NC_000004.12);
CP HBV: Hepatitis B virus core promoter (Quarleri J., World J Gastroenterol., 2014, 20, 425-435. (doi: 10.3748/wjg.v20.i2.425);
TTR: synthetic transthyretin promoter (TTR) comprising TTR enhancer (Samadani et al., Gene Expr., 1996, 6, 23-33) and human TTR promoter sequences (SEQ ID NO: 119);
HSE: synthetic promoter comprising HS-CR8 enhancer (Chuah et al., Mol Ther. 2014, 22, 1605-1613. doi: 10.1038/mt.2014.114) and human TTR promoter sequences (SEQ ID NO: 120);
Serpina1_comp. (SEQ ID NO: 121); synthetic alpha-1 antitrypsin promoter (SERPINA1 or AAT) corresponding to SEQ ID NO: 29 in WO 2020/104424.

A transgene plasmid containing the hFIX expression cassette flanked by AAV2 ITRs flanking was constructed for each hepatocyte-specific promoter. A transgene plasmid containing a CMV promoter-Luciferase reporter gene expression cassette flanked by AAV2 ITRs flanking was used as control.

### 2. AAV Production

HEK293T cells were grown in suspension in 50 mL of serum-free medium. The cells were transfected with 3 plasmids: i) a transgene plasmid, containing AAV2 ITRs flanking an expression cassette ii) the helper plasmid pXX6, containing adenoviral sequences necessary for AAV production, and iii) a plasmid containing AAV Rep and Cap genes, defining the serotype of AAV. Two days after transfection, the cells were lysed to release the AAV particles. The viral lysate was purified by affinity chromatography. Viral genomes were quantified by a TaqMan real-time PCR assay using primers and probes corresponding to the ITRs of the AAV vector genome (Rohr et al. J Virol Methods., 2002, 106,81-8.doi: 10.1016/s0166-0934 (02) 00138-6).

### 3. In Vivo Studies

All mouse studies were performed according to the French and European legislation on animal care and experimentation (2010/63/EU) and approved by the local institutional ethical committee (protocol no. 2016-002C). AAV vectors were administered intravenously via the tail vein to 6 weeks old male C57Bl6/J mice. PBS-injected littermates were used as controls.

### 4. Luciferase Activity

15 days after vector injections, tissues were harvested and homogenized in DNAse/RNAse free water using Fastprep tubes (6.5 m/s; 60 secondes). Luciferase assay was used to measure the expression of the reporter gene used as transgene. Tissue lysates were centrifuged at 10000 rpm for 10 min, the supernatant was diluted in lysis buffer in a white opaque 96-well plate. Luciferase activity was measured using EnSpire (PerkinElmer) through sequential injections of assay buffer containing ATP and luciferine. Protein quantification was performed on the samples using BCA assay in order to normalize the RLU (relative luminescence unit) on the quantity protein. The final results were expressed as RLU/mg of protein and normalized as fold change versus AAV8 control.

### 5. Factor IX Levels

Plasma levels of human FIX transgene were determined with an ELISA assay. The detection of hFIX antigen levels in mouse plasma was performed using the monoclonal antibodies against hFIX (GAFIX-AP, Affinity Biologicals, Ancaster, Canada) for coating and anti-hFIX HRP for detection (GAFIX-APHRP, Affinity Biologicals, Ancaster, Canada). The presence of hFIX antigen levels was determined by adding SIGMAFAST™ OPD substrate (Sigma-Aldrich) following manufacturer's instructions and measuring OD at an absorbance of 492 nm.

### 6. Vector Copy Number

Liver DNA was extracted from whole organ using the puregene cell and tissue kit (Qiagen) according to the manufacturer's instructions. Vector genome copy number (VGCN) was performed using qPCR with primers specific for hAAT promoter. P0 was used as normalizing gene. VGCN was quantified by TaqMan real-time PCR with the LightCycler480 II detector (Roche).

### Results

The design of capsids hereby described is based on the combination of the hypervariable regions (HVR) of two selected parental capsids: the well-known AAV8 serotype and the recently isolated WT AAV2/13 sequence (La Bella T et al., Gut., 2020, 69, 737-747.doi: 10.1136/gutjnl-2019-318281; WO2020/216861). The first group of mutants 5 (mut5) is composed by AAV8 from amino acid 1 to 446, isolated WT capsids from aa 447 to 485 (identified by #) and AAV8 from 486 to 739. These hybrids include all HVRs of AAV8 except the HVR5 from different WT capsids.
Mutant5-#508 (SEQ ID NO: 65) encoded by the polynucleotide of SEQ ID NO: 66), which comprises an HVR5 of SEQ ID NO: 44 encoded by the polynucleotide of SEQ ID NO: 67. SEQ ID NO: 44 is HVR5 sequence of AAV13.
Mutant5-#3142 (SEQ ID NO: 68) encoded by the polynucleotide of SEQ ID NO: 69, which comprises an HVR5 of SEQ ID NO: 45 encoded by the polynucleotide of SEQ ID NO: 70.
Mutant5-#M258 (SEQ ID NO: 71) encoded by the polynucleotide of SEQ ID NO: 72 which comprises an HVR5 of SEQ ID NO: 46 encoded by the polynucleotide of SEQ ID NO: 73.
Mutant5-#1570 (SEQ ID NO: 74) encoded by the polynucleotide of SEQ ID NO: 75, which comprises an HVR5 of SEQ ID NO: 47 encoded by the polynucleotide of SEQ ID NO: 76.
Mutant5-#2731 (SEQ ID NO: 77) encoded by the polynucleotide of SEQ ID NO: 78, which comprises an HVR5 of SEQ ID NO: 42 encoded by the polynucleotide of SEQ ID NO: 79.
Mutant5-#1602 (SEQ ID NO: 80) encoded by the polynucleotide of SEQ ID NO: 81, which comprises an HVR5 of SEQ ID NO: 48 encoded by the polynucleotide of SEQ ID NO: 82.
Mutant5-#667 (SEQ ID NO: 83) encoded by the polynucleotide of SEQ ID NO: 84, which comprises an HVR5 of SEQ ID NO: 41 encoded by the polynucleotide of SEQ ID NO: 85.
Mutant5-#129 (SEQ ID NO: 86) encoded by the polynucleotide of SEQ ID NO: 87), which comprises an HVR5 of SEQ ID NO: 43 encoded by the polynucleotide of SEQ ID NO: 88.

The second group of hybrid capsids (C8) is composed by AAV8 and a single hypervariable region from #704. The number of the mutated HVR is indicated by the number after the R. For example, the hybrid capsid C8-mut.R1 is AAV8 sequence, and the HVR1 sequence from the #704.
C8-mut.R1 (SEQ ID NO: 89) encoded by the polynucleotide of SEQ ID NO: 90;
C8-mut.R3 (SEQ ID NO: 91) encoded by the polynucleotide of SEQ ID N: 92;
C8-mut.R6 (SEQ ID NO: 93) encoded by the polynucleotide of SEQ ID NO: 94;
C8-mut.R7 (SEQ ID NO: 95) encoded by the polynucleotide of SEQ ID NO: 96;
C8-mut.R8 (SEQ ID NO: 97) encoded by the polynucleotide of SEQ ID NO: 98;
C8-mut.R9 (SEQ ID NO: 99) encoded by the polynucleotide of SEQ ID NO: 100;
C8-mut.R10 (SEQ ID NO: 101) encoded by the polynucleotide of SEQ ID NO: 102;
C8-mut.R11 (SEQ ID NO: 103) encoded by the polynucleotide of SEQ ID NO: 104; and
C8-mut.R12 (SEQ ID NO: 105) encoded by the polynucleotide of SEQ ID NO: 106.

The hybrid capsids have been produced with a hAAT hepatocyte-specific promoter followed by the secreted FIX transgene. AAV vectors were administered intravenously via the tail vein to 6 weeks old male C57Bl6 mice at a dose of 1×10¹⁰ vg/mouse. PBS-injected littermates were used as controls. 1 month after vector injections, sera and liver were harvested. FIX ELISA was used to quantify the FIX activity in blood, reflecting the transgene expression in hepatocytes. Results are expressed as μg of expressed FIX protein by mL of blood.

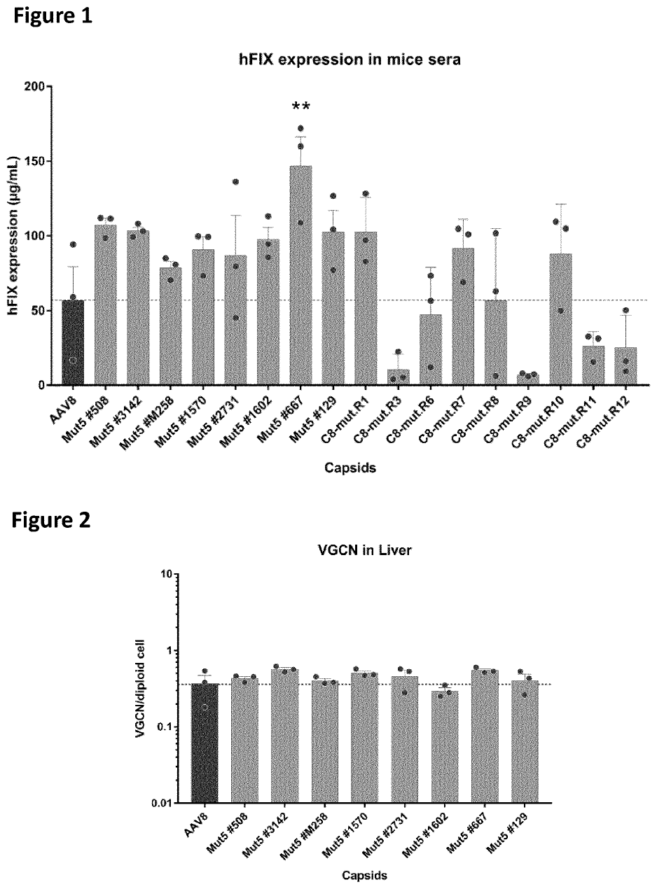

The FIX expression was higher in all the HVR5 mutant capsids compared to AAV8. Concerning the C8 hybrid capsids, there was a higher expression in the C8-mut.R1, C8-mut.R7 and C8-mut.R10 capsids (FIG. 1).

Then, the vector genome copy number per cell was assessed in hepatocytes (VGCN). The VGCN were analysed by qPCR, with primers designed on the hAAT promoter. The intercalant agent used to quantify the DNA was the SYBR green. The VGCN in hepatocytes was not significantly different between all hybrid capsids compared to AAV8, meaning that hybrid capsids transduced the liver at the same level of AAV8 control (FIG. 2).

Comparing the VGCN data and expression level, these results show that Mut5 hybrid capsids enhance the transgene expression without increasing the hepatocytes transduction, suggesting an important role of the capsid in the transgene expression.

The same hybrid capsids have also been produced with a CMV promoter followed by a firefly luciferase transgene. AAV vectors were administered intravenously via the tail vein to 6 weeks old male C57Bl6 mice at a dose of 1×10¹¹ vg/mouse. PBS-injected littermates were used as controls. 15 days after vector injections, liver was harvested.

Luciferase activity assay was used to indirectly quantify the expression of the reporter transgene in liver. Results are expressed as RLU (relative luminescence unit) per mg of protein and normalized as fold change versus AAV8 control.

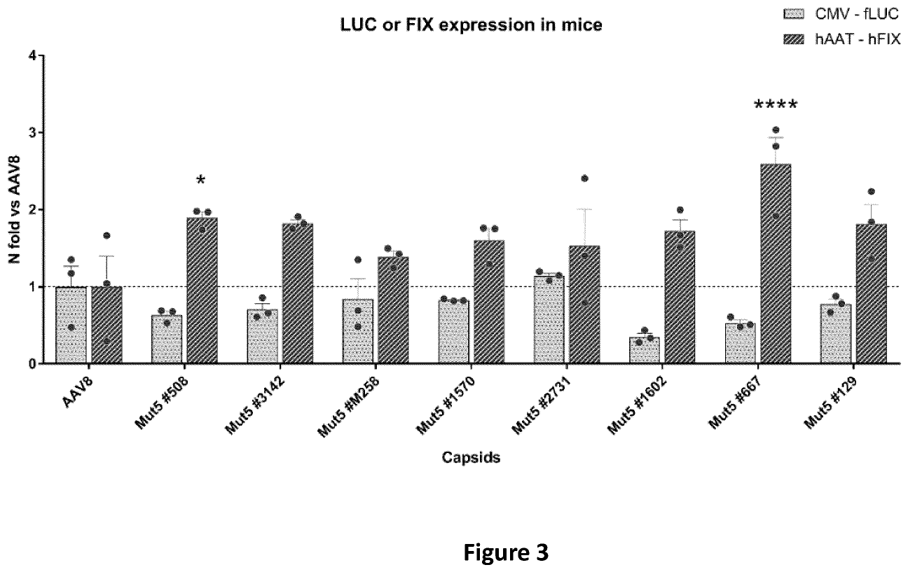

The results show a higher FIX expression of hybrid capsids compared to AAV8, whereas they display a lower LUC expression, meaning that the combination of the hybrid capsids with an hepatocyte-specific promoter enhances the transgene expression (FIG. 3).

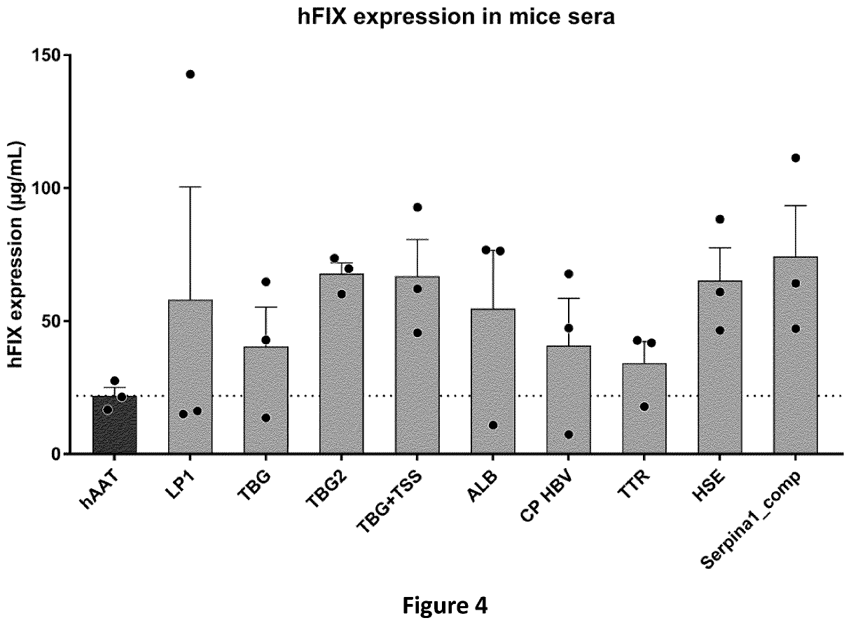

FIX expression levels were further enhanced when hAAT promoter was replaced with other hepatocyte-specific promoters: LP1, TBG, TBG2, TBG+TSS, ALB, CP HBV. TTR, HSE, and SERPINA1_COMP (FIG. 4).

Description of the sequences

| ID | | |
|---|---|---|
| NO | Name | Sequence |
| 1 | AAV8 | MAADGYLPDWLEDNLSEGIREWWALKPGAPKPKANQQKQDDGRGLVLPGYKYLGPFN |
| | | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLSRTQTTGG |
| | | TANTQTLGFSQGGPNTMANQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTENQSKLNSFITQYSTGQVSVE |
| | | IEWELQKENSKRWNPEIQYTSNYYKSTSVDFAVNTEGVYSEPRPIGTRYLTRNL |
| 2 | #704 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 3 | #129 | MAADGYLPDWLEDTLSEGIRQWWKLKPGPPPPKPAERHQDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPAEPDSSSGTGKAGQQPARKRL |
| | | NFGQTGDADSVPDPQPLGQPPAAPTSLGSTTMATGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFTFSYTFEDVPFHSSYAHSQSLDRLMNPLIDQYLYYLNRTQTXSGTPT |
| | | QSRLLFSQAGPTSMSLQAKNWLPGPCYRQQRLSKQANDNNNSNFPWTGATKYHLNGR |
| | | DSLVNPGPAMASHKDDEEKFFPMHGTLIFGKQGSEKTNAELENVMITDEEEIRTTNP |
| | | VATEQYGSVSTNLQRGNRQAATADVNTQGVLPGMVWQDRDVYLQGPIWAKIPHTDGH |
| | | FHPSPLMGGFGLKHPPPQILIKNTPVPANPSTTFSAAKFASFITQYSTGQVSVEIEW |
| | | ELQKENSKRWNPEIQYTSNYNKSVNVDFTVDINGVYSEARPIGTRYLTRNL |
| 4 | #2731 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEERVKTAPGKKRPVAVAHQWQTITRVPMEWVIPQEKRLN |
| | | FGQTGDADWHCDPQPLGQPPAAPSGLGSTTMATGSGALLLLESLSADGVGNSSGNWH |
| | | CDSQWLGDRVITTSTRTWALPTYNNHLYKQISSQSGASNDNHYFGYSTPWGYFDFNR |
| | | FHCHFSPRDWQRLINNNWGFRPKRLNFKLFNIQVKEVTQNDGTTTIANNLTSTVQVF |
| | | TDSEYQLPYVLGSAHQGCLPPFPADVFMVPQYGYLTLNNGSQAVGRSSFYCLEYFPS |
| | | QMLRTGNNFQFSYTFEDVPFHSSYAHSQSLDRLMNPLIDQYLYYLNKTQSNSGTTTM |
| | | SRLLFSQAGPTSMSLHAKNWLPGPCYRQQRLSKQANDNNNSNFPWTAATKYHLNGSD |
| | | SLINPGPAMASHKDDEEKFFPQSGVLIFGKQGSEKTNVDIEKVMITDEEEIRTTNPV |
| | | ATEQYGSVSTNLQRGNRRAATADVNTQGVLPGMVWQDRDVYLQGPIWAKIPHTDGHE |
| | | HPSPLMGGFGLKHPPPQILIKNTPVPANPSTTFSAAKFASFITQYSTGQVSVEIEWE |
| | | LQKENSKRWNPEIQYTSNYNKSVNVDFTVDINGVYSEPRPIGTRYLTRNL |
| 5 | #1602 | MAADGYLPDWLEDTLSEGIRQWWKLKPGPPPPKSAERHKDDSRGLVLPGYKYLGPFX |
| | | GLDKGEPVNEADAAALEHDKAYDRQLDSGDNPYLKYNHADAEFQERLKEDTSFGGNL |
| | | GRAVFQAKKRVLXPLGXVEEPVKTAPGKKRPVEHSPVEPDSSSGTGKAGQQPARKRL |
| | | NFGQTGDSDSSSRDMRKVFSAAAPTSLGSTTMASGGGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQTNSGTLQ |
| | | QSRLLFSQAGPTNMSLQAKNWLPGPCYRQQRLSKQANDNNNSNFPWTAATKYHLNGR |
| | | DSLVNPGPAMASHKDDEEKFFPQSGVLIFGKQGSEKTNVDIEKVMITDEEEIRTTNP |
| | | VATEQYGSVSTNLQRGNRQAATADVNTQGVLPGMVWQDRDVYLQGPIWAKIPHTDGH |
| | | FHPSPLMGGFGLKHPPPQILIKNTPVPANPSTTFSAAKFASFITQYSTGQVSVEIEW |
| | | ELQKEXSKRWNPEIQYTSNYNKSVNVDFTVDTNGVYSEPRPIGTRYLTRNL |
| 6 | #1010 | MAADGYLPDWLGETLSQGIRQWWKLKPGPPPPKPAERHKXDSRGLVLPGYKYLGPFN |
| | | GLDKGEPVNEADAAALEHDKAYDRQLDSGHIPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGTTTMATGSGAPVADNNEGADGVGNSSGNW |
| | | HCDSQWLGDRVITTSTRTWALPTYNNHLYNFFQIKINASNDNHYFGYSTPWGYFDFN |
| | | RFHCHFSPRDWQRLINNNWGFRPKRLNFKLFLIQVKEVTQNDGTTTIANNLTSTVQV |
| | | FTXSEYQLPYVLGSAHQGCXPPFPADVFMVPQYGYLTLNNGSQAXGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRVSKTSADNNNSEYSWTGATKYHLNGR |
| | | DSLVNPGPAMASHKDDEEKFFPQSGVLIFGKQGSEKTNVDIEKVMITDEEEIRTTNP |
| | | VATEQYGYVSNNLQNSNRQAATADVNTQGVLPGMVWQDRDVYLQGPIWAKIPHTDGH |
| | | FHPSPLMGGFGLKHPPPQILIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 7 | #667 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGTNTMATGSGAPMEDNNEGADGVGNSSGNW |
| | | HCDSQWLGDRVITTSTRTWALPTYNNHLYKQICSQSGASNDNHYFGYSTRWGDFDFN |
| | | RFHCHFSPRDWQRLINNNWGFRPKRLNFKLFNIQVKEVTQNDGTXTIANNLTSTVQV |
| | | FTDSEYQLPYVLGSAHQGCLPPFPADVFMVPQYGYLTLNNGSQAVGRSSFYCLEYFP |
| | | SQMLRTGNNFTFSYTFEDVPFHSSYAHSQSLDRLMNPLIDQYLYYLSRTNTPSGTTT |
| | | QSRLLFSQAGPTSMSLQAKNWLPGPCYRQQRLSKQANDNNNSNFPWTAATKYHLNGR |
| | | DSLVNPGPAMASHKDDEEKFFPQSGVLIFGKQGSEKTNVDIEKVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKSSXVPANPPTNFSSAKFASFITQYSTGQVSVEIEW |
| | | ELQKENSKRWNPEIQYTSNYNKSVNVDFTVDINGVYSEPRPIGTRYLTRNL |
| 8 | #767 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPAEPDSSSGTGKAGQQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNHYFGYSTPWGYFDFNRF |
| | | HCHFSPRDWQRLINNNWGFRPKRLNFKLFNIQVKEVTQNDGTTTIANNLTSTVQVFT |
| | | DSEYQLPYVLGSAHQGCLPPFPADVFMVPQYGYLTLNNGSQAVGRSSFYCLEYFPSQ |
| | | MLRTGNNFTFSYTFEDVPFHSSYAHSQSLDRLMNPLIDQYLYYLNKTQSSGTLQQSR |
| | | LLFSQAGPTSMSLQARNWLPGPCYRQQRVSKTSADKNNSEYSWTAATKYHLNGRDSL |
| | | VNPGPAMASHKDDEEKFFPMPGTLIFGKEGTNANNAELENVMITDEEEIRTTNPVAT |
| | | EQYGYVSNNLQNSNTGPTTGTVNHQGALPGMVWQDRDVYLQGPIWAKIPHTDGHFHP |
| | | SPLMGGFGLKHPPPQIMIKNTPVPANPPTNFSAAKFASFITQYSTGQVSVEIEWELQ |
| | | KENSKRWNPEIQYTSNYNKSVNVDFTVDINGVYSEPRPIGTRYLTRNL |
| 9 | #508 | MAADGYLPDWLEDTLSEGIRQWWKLKPGPPPPKPAERHKDDSRGLVLPGYKYLGPFN |
| | | GLDKGEPVNEADAAAIEHDKAYDRQLDSGDNPYLKYNHADAEFQERLKEDTSFGGNL |
| | | GRAVFQAKKRVLEXXGLVEEPVKTAPGKKRPVAHSPAEPDSSSGTGKAGNQPARKRL |
| | | NFGQTGDADSVPDPQPLGQPPAAPTSLGSTTMATGSGAPMADNNEGADGVGNSSGNW |
| | | HCDSTWMGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNRTQTASGTQQ |
| | | SRLLFSQAGPTSMSLQAKNWLPGPCYRQQRLSKQANDNNNSNYSWTGATKYHLNGRD |
| | | SLVXPGPAMASHXDDEXKFFPMHGTLIFGKEGTNATNADLDNVMITDEEEIRTTNPV |
| | | ATEQYGYVSNNLQNSNTAATTETVNHQGALPGMVWQDRDVYLQGPIWAKIPHTDGHF |
| | | HPSPLMGGFGLKNPPPQIMIKNTPVPANPPTNFSSAKFASFITQYSTGQVSVEIEWE |
| | | LQKENSKRWNPEIQYTSNYNKSVNVDFTVDINGVYSEPRPIGTRYLTRNL |
| 10 | #1704 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | ARAVFQAKKRVLEPLGLVEEPVKTAPGKKRPVEHSPAEPDSSSGTGKSGNQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFTFSYTFEDVPFHSSYAHSQSLDRLMNPLIDQYLYYLNKTQTNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLENVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 11 | #2112 | MAADGYLPDWLQENLFFDIRQWWKLKPGPPPPKPAERHQDNSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPAEPDSSSGTGKAGKQPAXKRL |
| | | NFGQTGXADSVPDPQPLGQPPAAPSGLGTNTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNKAGKRQQQQQLSLDCGY |
| | | KVSTKWPGPAMASHKDDEEKFFPMHGTLIFGKQGTNANDADLDNVMITDEEEIRTTN |
| | | PVATEQYGYVSNNLQNSNTGPTTGTVNHQGALPGMVWQDRDVYLQGPIWAKIPHTDG |
| | | HFHPSPLMGGFGLKHPPPQIMIKNTPVPANPPTNFSSAKFASFITQYSTGQVSVEIE |
| | | WELQKENSKRWNPEIQYTSNYNKSVNVDFTVDINGVYSEPRPIGTRYLTRNL |
| 12 | #1350 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQSGNTQAATGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 13 | #668 | MAADGYLPDWLEDTLSEGIRQWWKLKPGPPPPKPAERHKDDNRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 14 | #367 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 15 | #1020 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLENVMITDEEEIRTTNP |
| | | VATEQYGTVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 16 | #1158 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSEGGNL |
| | | GRAVFQAKKRVLEPLGLVEEPVKTAPGKKRPVEHSPVEPDSSSGTGKAGQQPARKRL |
| | | NFGQTGDADSVPDPQPLGKPPAAPSGLGSTTMATGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSSKFASFITQYSTGQVSVEIEW |
| | | ELQKENSKRWNPEIQYTSNYNKSVNVDFTVDINGVYSEPRPIGTRYLTRNL |
| 17 | #2107 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 18 | #1570 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQANSGTLQ |
| | | QSRLLFSQAGPTSMSLQAKNWLPGPCYRQQRLSKQANDNNNSNFPWTAATKYHLNGR |
| | | DSLVNPGPAMASHKDDEEEKFFPMHGTLIFGKQGTNANDADLDNVMITDEEEIRTTN |
| | | PVATEQYXYVSNNLQNSNTGPTTGTVNHQGALPGMVWQDRDVYLQGPIWAKIPHTDG |
| | | HFHPSPLMGGFGLKHPPPQIMIKNTPVPSPLMGGFGLKHPPPQITQYSTGQVSVEIE |
| | | WELQKENSKRWNPEIQYTSNYNKSVNVDFTVDINGVYSEPRPIGTRYLTRNL |
| 19 | #714 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPAEPDSSSGIGKAGQQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 20 | #3086 | MAADGYLPDW LEDTLSEGIR QWWKLKPGPP PPKPAERHKD NSRGLVLPGY |
| | | KYLGPFNGLD KGEPVNEADA AALEHDKAYD RQLDSGDNPY LKYNHADAEF |
| | | QERLKEDTSF GGNLGRAVFQ AKKRVLEPLG LVEEPVKTAP GKKRPVEHSP |
| | | VEPDSSSGTG KAGQQPAKKR LNFGQTGDAD SVPDPQPLGQ PPAAPSGLGS |
| | | TTMATGSGAP MADNNEGADG VGNSSGNWHC DSQWLGDRVI TTSTRTWALP |
| | | TYNNHLYKQI SSQSGASNDN HYFGYSTPWG YFDFNRFHCH FSPRDWQRLI |
| | | NNNWGFRPKR LNFKLFNIQV KEVTQNDGTT TIANNLTSTV QVFTDSEYQL |
| | | PYVLGSAHQG CLPPFPADVF MVPQYGYLTL NNGSQAVGRS SFYCLEYFPS |
| | | QMLRTGNNFQ FSYTFEDVPF HSSYAHSQSL DRLMNPLIDQ YLYYLNKTQS |
| | | NSGTLQQSRL LFSQAGPTNM SLQAKNWLPG PCYRQQRLSK QANDNNNSNF |
| | | PWTAATKYHL NGRDSLVNPG PAMASHKDDE EKFFPMHGTL IFGKQGTNAN |
| | | DADLDNVMIT DEEEIRTTNP VATEQYGYVS NNLQNSNTGP TTGTVNHQGA |
| | | LPGMVWQDRD VYLQGPIWAK IPHTDGHFHP SPLMGGFGLK HPPPQIMIKN |
| | | TPVPANPPTN FSSAKFASFI TQYSTGQVSV EIEWELQKEN SKRWNPEIQY |
| | | TSNYNKSVNV DFTVDINGVY SEPRPIGTRY LTRNL |
| 21 | #3142 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPAEPDSSSGTGKAGNQPARKRL |
| | | NFGQTGDADSVPDPQPIGQPPAAPTSLGSTTMATGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTVQ |
| | | QSRLLFSQAGPTSMSLQAKNWLPGPCYRQQRLSKQASDNNNSNFPWTAATKYHLNGR |
| | | DSLVNPGPAMASHKDDEEKFFPMHGTLIFGKEGTTSNNADLEHVMITDEEEIRTTNP |
| | | VATEQYGNVSNNLQNSNTGPTTENVNNQGALPGMVWQDRDVYLQGPIWAKIPHTDGH |
| | | FHPSPLMGGFGLKHPPPQIMIKNTPVPANPPTNFSAAKFASFITQYSTGQVSVEIEW |
| | | ELQKENSKRWNPEIQYTSNYNKSVNVDFTVDINGVYSEPRPIGTRYLTRNL |
| 22 | #1024 | MAADGYLPDWLEDNLSEGIREWWALKPGAPQPKANQQHQDNARGLVLPGYKYLGPGN |
| | | GLDKGEPVNAADAAALEHDKAYDKQLKAGDNPYLKYNHADAEFQERLKEDTSFGGNL |
| | | GRAVFQAKKRVLEPLGLVEEPVKTAPGKKRPVEHSPAEPDSSSGTGKSGQQPARKRL |
| | | NFGQTGDADSVPDPQPLGQPPAAPSGLGSTTMATGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNRTQTASGTQQ |
| | | SRLLFSQAGPTSMSLQAKNWLPGPCYRQQRLSKQANDNNNSNFPWTAATKYHLNGRD |
| | | SLVNPGPAMASHKDDEEKFFPMHGTLIFGKEGTNATNADLDNVMITDEEEIRTTNPV |
| | | ATEQYGYVSNNLQNSNTAATTETVNHQGALPGMVWQDRDVYLQGPIWAKIPHTDGHF |
| | | HPSPLMGGFGLKHPPPQIMIKNTPVPANPPTNFSAAKFASFITQYSTGQVSVEIEWE |
| | | LQKENSKRWNPEIQYTSNYNKSVNVDFTVDTNGVYSEPRPIGTRYLTRNL |
| 23 | #790 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGNQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDTNGVYSEPRPIGTRYLTRNL |
| 24 | #976 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 25 | #1286 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 26 | #163 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPVADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKKLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDTNGVYSEPRPIGTRYLTRNL |
| 27 | #685 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPAEPDSSSGTGKAGQQPARKRL |
| | | NFGQTGDADSVPDPQPLGQPPAAPSGLGSTTMATGSGAPMADNNEGADGVGNSSGNW |
| | | HCDSQWLGNRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTNANDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 28 | #442 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPVEPDSSSGTGKAGQQPARKRL |
| | | NFGQTGDADSVPDPQPLGQPPAAPSGLGSTTMATGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQANDNNNSNFPWTAATKYHLNGR |
| | | DSLVNPGPAMASHKDDEEKFFPMHGTLIFGKQGTGRDNADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 29 | #2320 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLETLGLVEEHVKTAPGKKRAVEHAHVEPDSSSGTGKAGQQPARKRL |
| | | NFGQTGDADSVPDPQPLGQPPAAPXGLGSTTMASGSGAPMADNNEGADGVGNSSGNW |
| | | HCDSQWLGDRVITTSTRTWALPTYNNHLYKQISSQSGASNDNHYFGYSTPWGYFDFN |
| | | RFHCHFSPRDWQRLINNNWGFRPKRLNFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNKTQSNSGTLQ |
| | | QSRLLFSQAGPTSMSLQAKNWLPGPCYRQQRLSKQATDNNNSEFPWTAATKYHLNGR |
| | | DSLVNPGPAMASHKDDEEKFFPMHGTLIFGKQGTKCNDADLDNVMITDEEEIRTTNP |
| | | VATEQYGYVSNNLQNSNTGPTTGTVNHQGALPGMVWQDRDVYLQGPIWAKIPHTDGH |
| | | FHPSPLMGGFGLKHPPPQIMIKNTPVPANPPTNFSSAKFASFITQYSTGQVSVEIEW |
| | | ELQKENSKRWNPEIQYTSNYNKSVNVDFTVDINGVYSEPRPIGTRYLTRNL |
| 30 | #M258 | MAADGYLPDWLEDTLSEGIRQWWKLKPGPPPPKPAERHKDDSRGLVLPGYKYLGPFN |
| | | GLDKGEPVNEADAAALEHDKAYDRQLDSGDNPYLKYNHADAEFQERLKEDTSFGGNL |
| | | GRAVFQAKKRVLEPLGLVEEPVKTAPGKKRPVEHSPAEPDSSSGTGKAGNQPARKRL |
| | | NFGQTGDADSVPDPQPLGEPPAAPTSLGSTTMASGGGAPVADNNEGADGVGNSSGKL |
| | | ALRFPMAGRQSHYHQHPNLGPAHLQQPSVQANFQPIRSQQRQPLILAHCNPWGYFDF |
| | | NRFHCHFSPRDWQRLINNNWGFRPKRLNFKLFNIQVKEVTQNDGTTTIANNLTSTVQ |
| | | VFTDSEYQIPYVLGSAHQGCLPPFPADVFMVPQYGYLTLNNGSQAVGRSSFYCLEYF |
| | | PSQMLRTGNNFQFSYTFEDVPFHSSYAHSQSLDRLMNPLIDQYLYYLNKTQSASGTV |
| | | QQSRLLFSQAGPTSMSLQAKNWLPGPCYRQQRLSKQANENNNSNFPWTAATKYHLNG |
| | | RDSLVNPGPAMASHKDDEEKFFPMHGTLIFGKQGTGRDNADLDNVMITDEEEIRTTN |
| | | PVATEQYGYVSNNLQNSNTAPSTGTVNHQGALPGMVWQDRDVYLQGPIWAKIPHTDG |
| | | HFHPSPLMGGFGLKHPPPQIMIKNTPVPANPPTNFSAAKFASFITQYSTGQVSVEIE |
| | | WELQKENSKRWNPEIQYTSNYNKSVNVDFTVDTNGVYSEPRPIGTRYLTRNL |
| 31 | AAV- | MAADGYLPDWLEDNLSEGIREWWALQPGAPKPKANQQHQDNARGLVLPGYKYLGPGN |
| | LK03 | GLDKGEPVNAADAAALEHDKAYDQQLKAGDNPYLKYNHADAEFQERLKEDTSFGGNL |
| | (WO2015/ | GRAVFQAKKRLLEPLGLVEEAAKTAPGKKRPVDQSPQEPDSSSGVGKSGKQPARKRL |
| | 121501) | NFGQTGDSESVPDPQPLGEPPAAPTSLGSNTMASGGGAPMADNNEGADGVGNSSGNW |
| | SEQ ID | HCDSQWLGDRVITTSTRTWALPTYNNHLYKQISSQSGASNDNHYFGYSTPWGYFDFN |
| | NO: 12 | RFHCHFSPRDWQRLINNNWGFRPKKLSFKLFNIQVKEVTQNDGTTTIANNLTSTVQV |
| | | FTDSEYQLPYVLGSAHQGCLPPFPADVFMVPQYGYLTLNNGSQAVGRSSFYCLEYFP |
| | | SQMLRTGNNFQFSYTFEDVPFHSSYAHSQSLDRLMNPLIDQYLYYLNRTQGTTSGTT |
| | | NQSRLLFSQAGPQSMSLQARNWLPGPCYRQQRLSKTANDNNNSNFPWTAASKYHLNG |
| | | RDSLVNPGPAMASHKDDEEKFFPMHGNLIFGKEGTTASNAELDNVMITDEEEIRTTN |
| | | PVATEQYGTVANNLQSSNTAPTTRTVNDQGALPGMVWQDRDVYLQGPIWAKIPHTDG |
| | | HFHPSPLMGGFGLKHPPPQIMIKNTPVPANPPTTFSPAKFASFITQYSTGQVSVEIE |
| | | WELQKENSKRWNPEIQYTSNYNKSVNVDFTVDINGVYSEPRPIGTRYLTRPL |
| 32 | AAV13 | MTDGYLPDWLEDNLSEGVREWWALQPGAPKPKANQQHQDNARGLVLPGYKYLGPGNG |
| | | LDKGEPVNAADAAALEHDKAYDQQLKAGDNPYLKYNHADAEFQERLQEDTSFGGNLG |
| | | RAVFQAKKRILEPLGLVEEAAKTAPGKKRPVEQSPAEPDSSSGIGKSGQQPARKRLN |
| | | FGQTGDTESVPDPQPLGQPPAAPSGVGSTTMASGGGAPMADNNEGADGVGNSSGNWH |
| | | CDSQWLGDRVITTSTRTWALPTYNNHLYKQISSQSGATNDNHYFGYSTPWGYFDFNR |
| | | FHCHFSPRDWQRLINNNWGFRPKRLNFKLFNIQVKEVTQNDGTTTIANNLTSTVQVF |
| | | TDSEYQLPYVLGSAHQGCLPPFPADVFMVPQYGYLTLNNGSQAVGRSSFYCLEYFPS |
| | | QMLRTGNNFQFSYTFEDVPFHSSYAHSQSLDRLMNPLIDQYLYYLNRTQTASGTQQS |
| | | RLLFSQAGPTSMSLQAKNWLPGPCYRQQRLSKQANDNNNSNFPWTGATKYHLNGRDS |
| | | LVNPGPAMASHKDDKEKFFPMHGTLIFGKEGTNANNADLENVMITDEEEIRTTNPVA |
| | | TEQYGTVSNNLQNSNAGPTTGTVNHQGALPGMVWQDRDVYLQGPIWAKIPHTDGHFH |
| | | PSPLMGGFGLKHPPPQIMIKNTPVPANPPTNFSAAKFASFITQYSTGQVSVEIEWEL |
| | | QKENSKRWNPEIQYTSNYNKSVNVDFTVDINGVYSEPRPIGTRYLTRNL |
| 33 | HVR1 | EPVKTAPGKKRPVEHSPVEPDSSSGTGKAGQ |
| | AAV2, | |
| | #704 | |
| 34 | HVR1 | EPVKTAPGKKRPVEHSPAEPDSSSGTGKAGQ |
| | #129 | |
| 35 | HVR1 | EPVKTAPGKKRPVAHSPAEPDSSSGTGKAGN |
| | #508 | |
| 36 | HVR1 | EPVKTAPGKKRPVEHSPAEPDSSSGTGKSGN |
| | #1704 | |
| 37 | HVR1 | EPVKTAPGKKRPVEHSPVEPDSSSGTGKAGN |
| | #790 | |
| 38 | HVR1 | EHVKTAPGKKRAVEHAHVEPDSSSGTGKAGQ |
| | #2320 | |
| 39 | HVR1 | EAAKTAPGKKRPVEQSPAEPDSSSGIGKSGQ |
| | AAV13 | |
| 40 | HVR1 | EPVKTAPGKKRPVEHSPAEPDSSSGTGKSGK |
| | #2112 | |
| 41 | HVR5 | YYLSRTNTPSGTTTQSRLLFSQAGPTSMSLQAKNWLPGP |
| | #667 | |
| 42 | HVR5 | YYLNKTQSNSGTTTMSRLLFSQAGPTSMSLHAKNWLPGP |
| | #2731 | |
| 43 | HVR5 | YYLNRTQTTSGTPTQSRLLFSQAGPTSMSLQAKNWLPGP |
| | #129 | |
| 44 | HVR5 | YYLNRTQTASGTQQSRLLFSQAGPTSMSLQAKNWLPGP |
| | #508 and | |
| | AAV13 | |
| 45 | HVR5 | YYLNKTQSNSGTVQQSRLLFSQAGPTSMSLQAKNWLPGP |
| | #3142 | |
| 46 | HVR5 | YYLNKTQSASGTVQQSRLLFSQAGPTSMSLQAKNWLPGP |
| | #M258 | |
| 47 | HVR5 | YYLNKTQANSGTLQQSRLLFSQAGPTSMSLQAKNWLPGP |
| | #1570 | |
| 48 | HVR5 | YYLNKTQTNSGTLQQSRLLFSQAGPTSMSLQAKNWLPGP |
| | #1602 | |
| 49 | HVR5 | YYLSRTNTPSGTTTQSRLQFSQAGASDIRDQSRNWLPGP |
| | AAV2 | |
| 50 | HVR7 | SEYSWTGATKYH |
| | AAV2 | |
| | and | |
| | #1010 | |
| 51 | HVR7 | SNFPWTGATKYH |
| | AAV13 | |
| 52 | HVR7 | SNFPWTAATKYH |
| | #704 | |
| 53 | HVR7 | SEYSWTAATKYH |
| | #767 | |
| 54 | HVR7 | SNYSWTGATKYH |
| | #508 | |
| 55 | HVR7 | SEFPWTAATKYH |
| | #2320 | |
| 56 | HVR10 | TEQYGSVSTNLQRGNRQAATADVNTQGVLPGMVWQDRD |
| | AAV2 | |
| | and #129 | |
| 57 | HVR10 | TEQYGTVSNNLQNSNAGPTTGTVNHQGALPGMVWQDRD |
| | AAV13 | |
| 58 | HVR10 | TEQYGYVSNNLQNSNTGPTTGTVNHQGALPGMVWQDRD |
| | #704 | |
| 59 | HVR10 | TEQYGSVSTNLQRGNRRAATADVNTQGVLPGMVWQDRD |
| | #2731 | |
| 60 | HVR10 | TEQYGYVSNNLQNSNRQAATADVNTQGVLPGMVWQDRD |
| | #1010 | |
| 61 | HVR10 | TEQYGYVSNNLQNSNTAATTETVNHQGALPGMVWQDRD |
| | #508 | |
| 62 | HVR10 | TEQYGYVSNNLQSGNTQAATGTVNHQGALPGMVWQDRD |
| | #1350 | |
| 63 | HVR10 | TEQYGNVSNNLQNSNTGPTTENVNNQGALPGMVWQDRD |
| | #3142 | |
| 64 | HVR10 | TEQYGYVSNNLQNSNTAPSTGTVNHQGALPGMVWQDRD |
| | #M258 | |
| 65 | Mutant5- | MAADGYLPDWLEDNLSEGIREWWALKPGAPKPKANQQKQDDGRGLVLPGYKYLGPFN |
| | #508 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLNRTQTASG |
| | | TQQSRLLFSQAGPTSMSLQAKNWLPGPCYRQQRVSTTTGQNNNSNFAWTAGTKYHLN |
| | | GRNSLANPGIAMATHKDDEERFFPSNGILIFGKQNAARDNADYSDVMLTSEEEIKTT |
| | | NPVATEEYGIVADNLQQQNTAPQIGTVNSQGALPGMVWQNRDVYLQGPIWAKIPHTD |
| | | GNFHPSPLMGGFGLKHPPPQILIKNTPVPADPPTTFNQSKINSFITQYSTGQVSVEI |
| | | EWELQKENSKRWNPEIQYTSNYYKSTSVDFAVNTEGVYSEPRPIGTRYLTRNL |
| 66 | Mutant5- | atggctgccgatggttatcttccagattggctcgaggacaacctctctgagggcatt |
| | #508 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtattatctaaacaggacacaaacagccagtgga |
| | | actcagcagtctcggctactgtttagccaagctggacccaccagcatgtctcttcaa |
| | | gctaaaaactggctgcctggaccttgctaccgccaacaacgcgtctcaacgacaacc |
| | | gggcaaaacaacaatagcaactttgcctggactgctgggaccaaataccatctgaat |
| | | ggaagaaattcattggctaatcctggcatcgctatggcaacacacaaagacgacgag |
| | | gagcgtttttttcccagtaacgggatcctgatttttggcaaacaaaatgctgccaga |
| | | gacaatgcggattacagcgatgtcatgctcaccagcgaggaagaaatcaaaaccact |
| | | aaccctgtggctacagaggaatacggtatcgtggcagataacttgcagcagcaaaac |
| | | acggctcctcaaattggaactgtcaacagccagggggccttacccggtatggtctgg |
| | | cagaaccgggacgtgtacctgcagggtcccatctgggccaagattcctcacacggac |
| | | ggcaacttccacccgtctccgctgatgggcggctttggcctgaaacatcctccgcct |
| | | cagatcctgatcaagaacacgcctgtacctgcggatcctccgaccaccttcaaccag |
| | | tcaaagctgaactctttcatcacgcaatacagcaccggacaggtcagcgtggaaatt |
| | | gaatgggagctgcagaaggaaaacagcaagcgctggaaccccgagatccagtacacc |
| | | tccaactactacaaatctacaagtgtggactttgctgttaatacagaaggcgtgtac |
| | | tctgaaccccgccccattggcacccgttacctcacccgtaatctgtaa |
| 67 | HVR5 | tattatctaaacaggacacaaacagccagtggaactcagcagtctcggctactgttt |
| | #508 and | agccaagctggacccaccagcatgtctcttcaagctaaaaactggctgcctggacct |
| | AAV13 | |
| 68 | Mutant5- | MAADGYLPDWLEDNLSEGIREWWALKPGAPKPKANQQKQDDGRGLVLPGYKYLGPFN |
| | #3142 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLNKTQSNSG |
| | | TVQQSRLLFSQAGPTSMSLQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYYKSTSVDFAVNTEGVYSEPRPIGTRYLTRNL |
| 69 | Mutant5- | atggctgccgatggttatcttccagattggctcgaggacaacctctctgagggcatt |
| | #3142 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtattatctgaacaagacacaatcaaatagtgga |
| | | actgttcagcagtctcggctactgtttagccaagctggacccaccagcatgtctctt |
| | | caagctaaaaactggctgcctggaccttgctaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 70 | HVR5 | tattatctgaacaagacacaatcaaatagtggaactgttcagcagtctcggctactg |
| | #3142 | tttagccaagctggacccaccagcatgtctcttcaagctaaaaactggctgcctgga |
| | | cct |
| 71 | Mutant5- | MAADGYLPDWLEDNLSEGIREWWALKPGAPKPKANQQKQDDGRGLVLPGYKYLGPFN |
| | #M258 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLNKTQSASG |
| | | TVQQSRLLFSQAGPTSMSLQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYYKSTSVDFAVNTEGVYSEPRPIGTRYLTRNL |
| 72 | Mutant5- | atggctgccgatggttatcttccagattggctcgaggacaacctctctgagggcatt |
| | #M258 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtattatctgaacaagacacaatcagccagtgga |
| | | actgttcagcagtctcggctactgtttagccaagctggacccaccagcatgtctctt |
| | | caagctaaaaactggctgcctggaccttgctaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 73 | HVR5- | tattatctgaacaagacacaatcagccagtggaactgttcagcagtctcggctactg |
| | #M258 | tttagccaagctggacccaccagcatgtctcttcaagctaaaaactggctgcctgga |
| | | cct |
| 74 | Mutant5- | MAADGYLPDWLEDNLSEGIREWWALKPGAPKPKANQQKQDDGRGLVLPGYKYLGPFN |
| | #1570 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLNKTQANSG |
| | | TLQQSRLLFSQAGPTSMSLQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYYKSTSVDFAVNTEGVYSEPRPIGTRYLTRNL |
| 75 | Mutant5- | atggctgccgatggttatcttccagattggctcgaggacaacctctctgagggcatt |
| | #1570 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtattatctgaacaaaacacaagcaaatagtgga |
| | | actcttcagcagtctcggctactgtttagtcaagctggacccaccagcatgtctctt |
| | | caagctaaaaactggctgcctggaccttgctaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 76 | HVR5- | tattatctgaacaaaacacaagcaaatagtggaactcttcagcagtctcggctactg |
| | #1570 | tttagtcaagctggacccaccagcatgtctcttcaagctaaaaactggctgcctgga |
| | | cct |
| 77 | Mutant5- | MAADGYLPDWLEDNLSEGIREWWALKPGAPKPKANQQKQDDGRGLVLPGYKYLGPFN |
| | #2731 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLNKTQSNSG |
| | | TTTMSRLLFSQAGPTSMSLHAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYYKSTSVDFAVNTEGVYSEPRPIGTRYLTRNL |
| 78 | Mutant5- | atggctgccgatggttatcttccagattggctcgaggacaacctctctgagggcatt |
| | #2731 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtattatctgaacaaaacacaatcaaatagtgga |
| | | accaccacgatgtcaaggctactgtttagtcaagctggacccaccagcatgtctctt |
| | | cacgctaaaaactggctgcctggaccttgctaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 79 | HVR5- | tattatctgaacaaaacacaatcaaatagtggaaccaccacgatgtcaaggctactg |
| | #2731 | tttagtcaagctggacccaccagcatgtctcttcacgctaaaaactggctgcctgga |
| | | cct |
| 80 | Mutant5- | MAADGYLPDWLEDNLSEGIREWWALKPGAPKPKANQQKQDDGRGLVLPGYKYLGPFN |
| | #1602 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLNKTQTNSG |
| | | TLQQSRLLFSQAGPTNMSLQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKLNSFITQYSTGQVSVE |
| | | IEWELQKENSKRWNPEIQYTSNYYKSTSVDFAVNTEGVYSEPRPIGTRYLTRNL |
| 81 | Mutant5- | atggctgccgatggttatcttccagattggctcgaggacaacctctctgagggcatt |
| | #1602 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtattatctgaacaagacacaaacaaatagtgga |
| | | actcttcagcagtctcggctactgtttagccaagctggaccaaccaacatgtctctt |
| | | caagctaaaaactggctgcctggaccttgctaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 82 | HVR55- | tattatctgaacaagacacaaacaaatagtggaactcttcagcagtctcggctactg |
| | #1602 | tttagccaagctggaccaaccaacatgtctcttcaagctaaaaactggctgcctgga |
| | | cct |
| 83 | Mutant5- | MAADGYLPDWLEDNLSEGIREWWALKPGAPKPKANQQKQDDGRGLVLPGYKYLGPFN |
| | #667 | GLDKGEPVNAADAAALEHDKAYDQQLQAGDNPYLRYNHADAEFQERLQEDTSEGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLSRTNTPSG |
| | | TTTQSRLLFSQAGPTSMSLQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYYKSTSVDFAVNTEGVYSEPRPIGTRYLTRNL |
| 84 | Mutant5- | atggctgccgatggttatcttccagattggctcgaggacaacctctctgagggcatt |
| | #667 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtattacttgagcagaacaaacactccaagtgga |
| | | accaccacgcagtctcggctactgtttagtcaagctggacccaccagcatgtctctt |
| | | caagctaaaaactggctgcctggaccttgctaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 85 | HVR55- | tattacttgagcagaacaaacactccaagtggaaccaccacgcagtctcggctactg |
| | #667 | tttagtcaagctggacccaccagcatgtctcttcaagctaaaaactggctgcctgga |
| | | cct |
| 86 | Mutant5- | MAADGYLPDWLEDNLSEGIREWWALKPGAPKPKANQQKQDDGRGLVLPGYKYLGPFN |
| | #129 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLNRTQTTSG |
| | | TPTQSRLLFSQAGPTSMSLQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYYKSTSVDFAVNTEGVYSEPRPIGTRYLTRNL |
| 87 | Mutant5- | atggctgccgatggttatcttccagattggctcgaggacaacctctctgagggcatt |
| | #129 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtattatctaaacagaacacaaacaAccagtgga |
| | | actcccacgcagtctcggctactgtttagccaagctggacccacaagcatgtctctt |
| | | caagctaaaaactggctgcctggaccgtgttaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 88 | HVR55- | tattatctaaacagaacacaaacaAccagtggaactcccacgcagtctcggctactg |
| | #129 | tttagccaagctggacccacaagcatgtctcttcaagctaaaaactggctgcctgga |
| | | ccg |
| 89 | C8- | MAADGYLPDWLEDNLSEGIREWWALKPGAPKPKANQQKQDDGRGLVLPGYKYLGPFN |
| | mut.R1 | GLDKGEPVNAADAAALEHDKAYDQQLQAGDNPYLRYNHADAEFQERLQEDTSFGGNL |
| | | GRAVFQAKKRVLEPLGLVEEPVKTAPGKKRPVEHSPVEPDSSSGTGKAGQQPARKRL |
| | | NFGQTGDSESVPDPQPLGEPPAAPSGVGPNTMAAGGGAPMADNNEGADGVGSSSGNW |
| | | HCDSTWLGDRVITTSTRTWALPTYNNHLYKQISNGTSGGATNDNTYFGYSTPWGYFD |
| | | FNRFHCHFSPRDWQRLINNNWGFRPKRLSFKLFNIQVKEVTQNEGTKTIANNLTSTI |
| | | QVFTDSEYQLPYVLGSAHQGCLPPFPADVFMIPQYGYLTLNNGSQAVGRSSFYCLEY |
| | | FPSQMLRTGNNFQFTYTFEDVPFHSSYAHSQSLDRLMNPLIDQYLYYLSRTQTTGGT |
| | | ANTQTLGFSQGGPNTMANQAKNWLPGPCYRQQRVSTTTGQNNNSNFAWTAGTKYHLN |
| | | GRNSLANPGIAMATHKDDEERFFPSNGILIFGKQNAARDNADYSDVMLTSEEEIKTT |
| | | NPVATEEYGIVADNLQQQNTAPQIGTVNSQGALPGMVWQNRDVYLQGPIWAKIPHTD |
| | | GNFHPSPLMGGFGLKHPPPQILIKNTPVPADPPTTFNQSKINSFITQYSTGQVSVEI |
| | | EWELQKENSKRWNPEIQYTSNYYKSTSVDFAVNTEGVYSEPRPIGTRYLTRNL |
| 90 | C8- | atggctgccgatggttatcttccagattggctcgaggacaacctctctgagggcatt |
| | mut.R1 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaacctgttaagacggctccgggaaaaaagaggccggtagagcactctcctgtggag |
| | | ccagactcctcctcgggaaccggaaaagcgggccaacagcccgccagaaaaagactc |
| | | aattttggtcagactggcgactcagagtcagttccagaccctcaacctctcggagaa |
| | | cctccagcagcgccctctggtgtgggacctaatacaatggctgcaggcggtggcgca |
| | | ccaatggcagacaataacgaaggcgccgacggagtgggtagttcctcgggaaattgg |
| | | cattgcgattccacatggctgggcgacagagtcatcaccaccagcacccgaacctgg |
| | | gccctgcccacctacaacaaccacctctacaagcaaatctccaacgggacatcggga |
| | | ggagccaccaacgacaacacctacttcggctacagcaccccctgggggtattttgac |
| | | tttaacagattccactgccacttttcaccacgtgactggcagcgactcatcaacaac |
| | | aactggggattccggcccaagagactcagcttcaagctcttcaacatccaggtcaag |
| | | gaggtcacgcagaatgaaggcaccaagaccatcgccaataacctcaccagcaccatc |
| | | caggtgtttacggactcggagtaccagctgccgtacgttctcggctctgcccaccag |
| | | ggctgcctgcctccgttcccggcggacgtgttcatgattccccagtacggctaccta |
| | | acactcaacaacggtagtcaggccgtgggacgctcctccttctactgcctggaatac |
| | | tttccttcgcagatgctgagaaccggcaacaacttccagtttacttacaccttcgag |
| | | gacgtgcctttccacagcagctacgcccacagccagagcttggaccggctgatgaat |
| | | cctctgattgaccagtacctgtactacttgtctcggactcaaacaacaggaggcacg |
| | | gcaaatacgcagactctgggcttcagccaaggtgggcctaatacaatggccaatcag |
| | | gcaaagaactggctgccaggaccctgttaccgccaacaacgcgtctcaacgacaacc |
| | | gggcaaaacaacaatagcaactttgcctggactgctgggaccaaataccatctgaat |
| | | ggaagaaattcattggctaatcctggcatcgctatggcaacacacaaagacgacgag |
| | | gagcgtttttttcccagtaacgggatcctgatttttggcaaacaaaatgctgccaga |
| | | gacaatgcggattacagcgatgtcatgctcaccagcgaggaagaaatcaaaaccact |
| | | aaccctgtggctacagaggaatacggtatcgtggcagataacttgcagcagcaaaac |
| | | acggctcctcaaattggaactgtcaacagccagggggccttacccggtatggtctgg |
| | | cagaaccgggacgtgtacctgcagggtcccatctgggccaagattcctcacacggac |
| | | ggcaacttccacccgtctccgctgatgggcggctttggcctgaaacatcctccgcct |
| | | cagatcctgatcaagaacacgcctgtacctgcggatcctccgaccaccttcaaccag |
| | | tcaaagctgaactctttcatcacgcaatacagcaccggacaggtcagcgtggaaatt |
| | | gaatgggagctgcagaaggaaaacagcaagcgctggaaccccgagatccagtacacc |
| | | tccaactactacaaatctacaagtgtggactttgctgttaatacagaaggcgtgtac |
| | | tctgaaccccgccccattggcacccgttacctcacccgtaatctgtaa |
| 91 | C8- | MAADGYLPDWLEDNLSEGIREWWALKPGAPKPKANQQKQDDGRGLVLPGYKYLGPFN |
| | mut.R3 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISSQSGASNDNHYFGYSTPWGYFDF |
| | | NRFHCHFSPRDWQRLINNNWGFRPKRLSFKLFNIQVKEVTQNEGTKTIANNLTSTIQ |
| | | VFTDSEYQLPYVLGSAHQGCLPPFPADVFMIPQYGYLTLNNGSQAVGRSSFYCLEYF |
| | | PSQMLRTGNNFQFTYTFEDVPFHSSYAHSQSLDRLMNPLIDQYLYYLSRTQTTGGTA |
| | | NTQTLGFSQGGPNTMANQAKNWLPGPCYRQQRVSTTTGQNNNSNFAWTAGTKYHLNG |
| | | RNSLANPGIAMATHKDDEERFFPSNGILIFGKQNAARDNADYSDVMLTSEEEIKTTN |
| | | PVATEEYGIVADNLQQQNTAPQIGTVNSQGALPGMVWQNRDVYLQGPIWAKIPHTDG |
| | | NFHPSPLMGGFGLKHPPPQILIKNTPVPADPPTTFNQSKINSFITQYSTGQVSVEIE |
| | | WELQKENSKRWNPEIQYTSNYYKSTSVDFAVNTEGVYSEPRPIGTRYLTRNL |
| 92 | C8- | atggctgccgatggttatcttccagattggctcgaggacaacctctctgagggcatt |
| | mut.R3 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccagccaatcagga |
| | | gcctcaaacgacaaccactacttcggctacagcaccccctgggggtattttgacttt |
| | | aacagattccactgccacttttcaccacgtgactggcagcgactcatcaacaacaac |
| | | tggggattccggcccaagagactcagcttcaagctcttcaacatccaggtcaaggag |
| | | gtcacgcagaatgaaggcaccaagaccatcgccaataacctcaccagcaccatccag |
| | | gtgtttacggactcggagtaccagctgccgtacgttctcggctctgcccaccagggc |
| | | tgcctgcctccgttcccggcggacgtgttcatgattccccagtacggctacctaaca |
| | | ctcaacaacggtagtcaggccgtgggacgctcctccttctactgcctggaatacttt |
| | | ccttcgcagatgctgagaaccggcaacaacttccagtttacttacaccttcgaggac |
| | | gtgcctttccacagcagctacgcccacagccagagcttggaccggctgatgaatcct |
| | | ctgattgaccagtacctgtactacttgtctcggactcaaacaacaggaggcacggca |
| | | aatacgcagactctgggcttcagccaaggtgggcctaatacaatggccaatcaggca |
| | | aagaactggctgccaggaccctgttaccgccaacaacgcgtctcaacgacaaccggg |
| | | caaaacaacaatagcaactttgcctggactgctgggaccaaataccatctgaatgga |
| | | agaaattcattggctaatcctggcatcgctatggcaacacacaaagacgacgaggag |
| | | cgtttttttcccagtaacgggatcctgatttttggcaaacaaaatgctgccagagac |
| | | aatgcggattacagcgatgtcatgctcaccagcgaggaagaaatcaaaaccactaac |
| | | cctgtggctacagaggaatacggtatcgtggcagataacttgcagcagcaaaacacg |
| | | gctcctcaaattggaactgtcaacagccagggggccttacccggtatggtctggcag |
| | | aaccgggacgtgtacctgcagggtcccatctgggccaagattcctcacacggacggc |
| | | aacttccacccgtctccgctgatgggcggctttggcctgaaacatcctccgcctcag |
| | | atcctgatcaagaacacgcctgtacctgcggatcctccgaccaccttcaaccagtca |
| | | aagctgaactctttcatcacgcaatacagcaccggacaggtcagcgtggaaattgaa |
| | | tgggagctgcagaaggaaaacagcaagcgctggaaccccgagatccagtacacctcc |
| | | aactactacaaatctacaagtgtggactttgctgttaatacagaaggcgtgtactct |
| | | gaaccccgccccattggcacccgttacctcacccgtaatctgtaa |
| 93 | C8- | MAADGYLPDWLEDNLSEGIREWWALKPGAPKPKANQQKQDDGRGLVLPGYKYLGPFN |
| | mut.R6 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLSRTQTTGG |
| | | TANTQTLGFSQGGPNTMANQAKNWLPGPCYRQQRLSKQANDNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTENQSKLNSFITQYSTGQVSVE |
| | | IEWELQKENSKRWNPEIQYTSNYYKSTSVDFAVNTEGVYSEPRPIGTRYLTRNL |
| 94 | C8- | atggctgccgatggttatcttccagattggctcgaggacaacctctctgagggcatt |
| | mut.R6 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtactacttgtctcggactcaaacaacaggaggc |
| | | acggcaaatacgcagactctgggcttcagccaagcggggcctaatacaatggccaat |
| | | caggcaaagaactggctgccaggaccctgctacagacagcagcgtctgtcaaagcag |
| | | gcaaacgacaacaacaacagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 95 | C8- | MAADGYLPDWLEDNLSEGIREWWALKPGAPKPKANQQKQDDGRGLVLPGYKYLGPFN |
| | mut.R7 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLSRTQTTGG |
| | | TANTQTLGFSQGGPNTMANQAKNWLPGPCYRQQRVSTTTGQNNNSNFPWTAATKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKLNSFITQYSTGQVSVE |
| | | IEWELQKENSKRWNPEIQYTSNYYKSTSVDFAVNTEGVYSEPRPIGTRYLTRNL |
| 96 | C8- | atggctgccgatggttatcttccagattggctcgaggacaacctctctgagggcatt |
| | mut.R7 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtactacttgtctcggactcaaacaacaggaggc |
| | | acggcaaatacgcagactctgggcttcagccaaggtgggcctaatacaatggccaat |
| | | caggcaaagaactggctgccaggaccctgttaccgccaacaacgcgtctcaacgaca |
| | | accgggcaaaacaacaatagcaactttccctggactgcggctacaaagtatcatcta |
| | | aatggccggaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 97 | C8- | MAADGYLPDWLEDNLSEGIREWWALKPGAPKPKANQQKQDDGRGLVLPGYKYLGPFN |
| | mut.R8 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLSRTQTTGG |
| | | TANTQTLGFSQGGPNTMANQAKNWLPGPCYRQQRVSTTTGQNNNSNFAWTAGTKYHL |
| | | NGRDSLVNPGPAMASHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYYKSTSVDFAVNTEGVYSEPRPIGTRYLTRNL |
| 98 | C8- | atggctgccgatggttatcttccagattggctcgaggacaacctctctgagggcatt |
| | mut.R8 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtactacttgtctcggactcaaacaacaggaggc |
| | | acggcaaatacgcagactctgggcttcagccaaggtgggcctaatacaatggccaat |
| | | caggcaaagaactggctgccaggaccctgttaccgccaacaacgcgtctcaacgaca |
| | | accgggcaaaacaacaatagcaactttgcctggactgctgggacaaagtatcatcta |
| | | aatggccgggactcgttggttaatccaggaccagctatggccagccacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 99 | C8- | MAADGYLPDWLEDNLSEGIREWWALKPGAPKPKANQQKQDDGRGLVLPGYKYLGPFN |
| | mut.R9 | GLDKGEPVNAADAAALEHDKAYDQQLQAGDNPYLRYNHADAEFQERLQEDTSEGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLSRTQTTGG |
| | | TANTQTLGFSQGGPNTMANQAKNWLPGPCYRQQRVSTTTGQNNNSNFAWTAGTKYHL |
| | | NGRNSLANPGIAMATHKDDEEKFFPMHGTLIFGKQGTNANDADLDNVMITDEEEIRT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYYKSTSVDFAVNTEGVYSEPRPIGTRYLTRNL |
| 100 | C8- | atggctgccgatggttatcttccagattggctcgaggacaacctctctgagggcatt |
| | mut.R9 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtactacttgtctcggactcaaacaacaggaggc |
| | | acggcaaatacgcagactctgggcttcagccaaggtgggcctaatacaatggccaat |
| | | caggcaaagaactggctgccaggaccctgttaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgat |
| | | gaagaaaagtttttccccatgcatggaaccctgatatttggtaaacaaggaacaaat |
| | | gctaacgacgcggatttggacaatgtcatgattacagatgaagaagaaatccgcacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 101 | C8- | MAADGYLPDWLEDNLSEGIREWWALKPGAPKPKANQQKQDDGRGLVLPGYKYLGPFN |
| | mut.R10 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLSRTQTTGG |
| | | TANTQTLGFSQGGPNTMANQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEQYGYVSNNLQNSNTGPTTGTVNHQGALPGMVWQDRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYYKSTSVDFAVNTEGVYSEPRPIGTRYLTRNL |
| 102 | C8- | atggctgccgatggttatcttccagattggctcgaggacaacctctctgagggcatt |
| | mut.R10 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtactacttgtctcggactcaaacaacaggaggc |
| | | acggcaaatacgcagactctgggcttcagccaaggtgggcctaatacaatggccaat |
| | | caggcaaagaactggctgccaggaccctgttaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacggagcagtacggatatgtgtcaaataatttgcaaaactca |
| | | aatactggtccaactactggaactgtcaatcaccaaggagcgttacctggtatggtg |
| | | tggcaggatcgagacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 103 | C8- | MAADGYLPDWLEDNLSEGIREWWALKPGAPKPKANQQKQDDGRGLVLPGYKYLGPFN |
| | mut.R11 | GLDKGEPVNAADAAALEHDKAYDQQLQAGDNPYLRYNHADAEFQERLQEDTSFGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLSRTQTTGG |
| | | TANTQTLGFSQGGPNTMANQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGHFHPSPLMGGFGLKHPPPQIMIKNTPVPANPPTNFSSAKFASFITQYSTGQVSVE |
| | | IEWELQKENSKRWNPEIQYTSNYYKSTSVDFAVNTEGVYSEPRPIGTRYLTRNL |
| 104 | C8- | atggctgccgatggttatcttccagattggctcgaggacaacctctctgagggcatt |
| | mut.R11 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtactacttgtctcggactcaaacaacaggaggc |
| | | acggcaaatacgcagactctgggcttcagccaaggtgggcctaatacaatggccaat |
| | | caggcaaagaactggctgccaggaccctgttaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacc |
| | | gatggacactttcatccttctccactgatgggaggttttgggctcaaacacccgcct |
| | | cctcagatcatgatcaaaaacactcccgttccagccaatcctcctacaaacttcagt |
| | | tctgccaagtttgcttctttcatcacacagtattccacggggcaggtcagcgtggag |
| | | atcgagtgggagctgcagaaggaaaacagcaagcgctggaaccccgagatccagtac |
| | | acctccaactactacaaatctacaagtgtggactttgctgttaatacagaaggcgtg |
| | | tactctgaaccccgccccattggcacccgttacctcacccgtaatctgtaa |
| 105 | C8- | MAADGYLPDWLEDNLSEGIREWWALKPGAPKPKANQQKQDDGRGLVLPGYKYLGPFN |
| | mut.R12 | GLDKGEPVNAADAAALEHDKAYDQQLQAGDNPYLRYNHADAEFQERLQEDTSEGGNL |
| | | GRAVFQAKKRVLEPLGLVEEGAKTAPGKKRPVEPSPQRSPDSSTGIGKKGQQPARKR |
| | | LNFGQTGDSESVPDPQPLGEPPAAPSGVGPNTMAAGGGAPMADNNEGADGVGSSSGN |
| | | WHCDSTWLGDRVITTSTRTWALPTYNNHLYKQISNGTSGGATNDNTYFGYSTPWGYF |
| | | DFNRFHCHFSPRDWQRLINNNWGFRPKRLSFKLFNIQVKEVTQNEGTKTIANNLTST |
| | | IQVFTDSEYQLPYVLGSAHQGCLPPFPADVFMIPQYGYLTLNNGSQAVGRSSFYCLE |
| | | YFPSQMLRTGNNFQFTYTFEDVPFHSSYAHSQSLDRLMNPLIDQYLYYLSRTQTTGG |
| | | TANTQTLGFSQGGPNTMANQAKNWLPGPCYRQQRVSTTTGQNNNSNFAWTAGTKYHL |
| | | NGRNSLANPGIAMATHKDDEERFFPSNGILIFGKQNAARDNADYSDVMLTSEEEIKT |
| | | TNPVATEEYGIVADNLQQQNTAPQIGTVNSQGALPGMVWQNRDVYLQGPIWAKIPHT |
| | | DGNFHPSPLMGGFGLKHPPPQILIKNTPVPADPPTTFNQSKINSFITQYSTGQVSVE |
| | | IEWELQKENSKRWNPEIQYTSNYNKSVNVDFTVDTNGVYSEPRPIGTRYLTRNL |
| 106 | C8- | atggctgccgatggttatcttccagattggctcgaggacaacctctctgagggcatt |
| | mut.R12 | cgcgagtggtgggcgctgaaacctggagccccgaagcccaaagccaaccagcaaaag |
| | | caggacgacggccggggtctggtgcttcctggctacaagtacctcggacccttcaac |
| | | ggactcgacaagggggagcccgtcaacgcggcggacgcagcggccctcgagcacgac |
| | | aaggcctacgaccagcagctgcaggcgggtgacaatccgtacctgcggtataaccac |
| | | gccgacgccgagtttcaggagcgtctgcaagaagatacgtcttttgggggcaacctc |
| | | gggcgagcagtcttccaggccaagaagcgggttctcgaacctctcggtctggttgag |
| | | gaaggcgctaagacggctcctggaaagaagagaccggtagagccatcaccccagcgt |
| | | tctccagactcctctacgggcatcggcaagaaaggccaacagcccgccagaaaaaga |
| | | ctcaattttggtcagactggcgactcagagtcagttccagaccctcaacctctcgga |
| | | gaacctccagcagcgccctctggtgtgggacctaatacaatggctgcaggcggtggc |
| | | gcaccaatggcagacaataacgaaggcgccgacggagtgggtagttcctcgggaaat |
| | | tggcattgcgattccacatggctgggcgacagagtcatcaccaccagcacccgaacc |
| | | tgggccctgcccacctacaacaaccacctctacaagcaaatctccaacgggacatcg |
| | | ggaggagccaccaacgacaacacctacttcggctacagcaccccctgggggtatttt |
| | | gactttaacagattccactgccacttttcaccacgtgactggcagcgactcatcaac |
| | | aacaactggggattccggcccaagagactcagcttcaagctcttcaacatccaggtc |
| | | aaggaggtcacgcagaatgaaggcaccaagaccatcgccaataacctcaccagcacc |
| | | atccaggtgtttacggactcggagtaccagctgccgtacgttctcggctctgcccac |
| | | cagggctgcctgcctccgttcccggcggacgtgttcatgattccccagtacggctac |
| | | ctaacactcaacaacggtagtcaggccgtgggacgctcctccttctactgcctggaa |
| | | tactttccttcgcagatgctgagaaccggcaacaacttccagtttacttacaccttc |
| | | gaggacgtgcctttccacagcagctacgcccacagccagagcttggaccggctgatg |
| | | aatcctctgattgaccagtacctgtactacttgtctcggactcaaacaacaggaggc |
| | | acggcaaatacgcagactctgggcttcagccaaggtgggcctaatacaatggccaat |
| | | caggcaaagaactggctgccaggaccctgttaccgccaacaacgcgtctcaacgaca |
| | | accgggcaaaacaacaatagcaactttgcctggactgctgggaccaaataccatctg |
| | | aatggaagaaattcattggctaatcctggcatcgctatggcaacacacaaagacgac |
| | | gaggagcgtttttttcccagtaacgggatcctgatttttggcaaacaaaatgctgcc |
| | | agagacaatgcggattacagcgatgtcatgctcaccagcgaggaagaaatcaaaacc |
| | | actaaccctgtggctacagaggaatacggtatcgtggcagataacttgcagcagcaa |
| | | aacacggctcctcaaattggaactgtcaacagccagggggccttacccggtatggtc |
| | | tggcagaaccgggacgtgtacctgcagggtcccatctgggccaagattcctcacacg |
| | | gacggcaacttccacccgtctccgctgatgggcggctttggcctgaaacatcctccg |
| | | cctcagatcctgatcaagaacacgcctgtacctgcggatcctccgaccaccttcaac |
| | | cagtcaaagctgaactctttcatcacgcaatacagcaccggacaggtcagcgtggaa |
| | | attgaatgggagctgcagaaggagaacagcaaacgctggaatcccgagattcagtac |
| | | acttccaactacaacaaatctgttaatgtggactttactgtggacactaatggtgtg |
| | | tattcagagcctcgccccattggcaccagatacctgactcgtaatctgtaa |
| 107 | hAAT | gatcttgctaccagtggaacagccactaaggattctgcagtgagagcagagggcca |
| | promoter | gctaagtggtactctcccagagactgtctgactcacgccaccccctccaccttgga |
| | | cacaggacgctgtggtttctgagccaggtacaatgactcctttcggtaagtgcagt |
| | | ggaagctgtacactgcccaggcaaagcgtccgggcagcgtaggcgggcgactcaga |
| | | tcccagccagtggacttagcccctgtttgctcctccgataactggggtgaccttgg |
| | | ttaatattcaccagcagcctcccccgttgcccctctggatccactgcttaaatacg |
| | | gacgaggacagggccctgtctcctcagcttcaggcaccaccactgacctgggacag |
| | | tgaat |
| 108 | Human | aggctcagaggcacacaggagtttctgggctcaccctgcccccttccaacccctcag |
| | ApoE | ttcccatcctccagcagctgtttgtgtgctgcctctgaagtccacactgaacaaact |
| | enhancer | tcagcctactcatgtccctaaaatgggcaaacattgcaagcagcaaacagcaaacac |
| | | acagccctccctgcctgctgaccttggagctggggcagaggtcagagacctctctgg |
| | | gcccatgccacctccaacatccactcgaccccttggaatttcggtggagaggagcag |
| | | aggttgtcctggcgtggtttaggtagtgtgagaggg |
| 109 | HBB2 | gtacacatat tgaccaaatc agggtaattt tgcatttgta attttaaaaa atgctttctt |
| | intron | cttttaatat acttttttgt ttatcttatt tctaatactt tccctaatct ctttctttca |
| | | gggcaataat gatacaatgt atcatgcctc tttgcaccat tctaaagaat aacagtgata |
| | | atttctgggt taaggcaata gcaatatttc tgcatataaa tatttctgca tataaattgt |
| | | aactgatgta agaggtttca tattgctaat agcagctaca atccagctac cattctgctt |
| | | ttattttatg gttgggataa ggctggatta ttctgagtcc aagctaggcc cttttgctaa |
| | | tcatgttcat acctcttatc ttcctcccac agctcctggg caacgtgctg gtctgtgtgc |
| | | tggcccatca ctttggcaaa g |
| 110 | Modified | gtacacatat tgaccaaatc agggtaattt tgcatttgta attttaaaaa atgctttctt |
| | HBB2 | cttttaatat acttttttgt ttatcttatt tctaatactt tccctaatct ctttctttca |
| | intron | gggcaataat gatacaatgt atcatgcctc tttgcaccat tctaaagaat aacagtgata |
| | | atttctgggt taaggcaata gcaatatttc tgcatataaa tatttctgca tataaattgt |
| | | aactgatgta agaggtttca tattgctaat agcagctaca atccagctac cattctgctt |
| | | ttattttctg gttgggataa ggctggatta ttctgagtcc aagctaggcc cttttgctaa |
| | | tcttgttcat acctcttatc ttcctcccac agctcctggg caacctgctg gtctctctgc |
| | | tggcccatca ctttggcaaa g |
| 111 | FIX | ggtttgtttc cttttttaaa atacattgag tatgcttgcc ttttagatat agaaatatct |
| | intron | gatgctgtct tcttcactaa attttgatta catgatttga cagcaatatt gaagagtcta |
| | | acagccagca cgcaggttgg taagtactgg ttctttgtta gctaggtttt cttcttcttc |
| | | atttttaaaa ctaaatagat cgacaatgct tatgatgcat ttatgtttaa taaacactgt |
| | | tcagttcatg atttggtcat gtaattcctg ttagaaaaca ttcatctcct tggtttaaaa |
| | | aaattaaaag tgggaaaaca aagaaatagc agaatatagt gaaaaaaaat aaccacatta |
| | | tttttgtttg gacttaccac tttgaaatca aaatgggaaa caaaagcaca aacaatggcc |
| | | ttatttacac aaaaagtctg attttaagat atatgacatt tcaaggtttc agaagtatgt |
| | | aatgaggtgt gtctctaatt ttttaaatta tatatcttca atttaaagtt ttagttaaaa |
| | | cataaagatt aacctttcat tagcaagctg ttagttatca ccaacgcttt tcatggatta |
| | | ggaaaaaatc attttgtctc tatgtcaaac atcttggagt tgatatttgg ggaaacacaa |
| | | tactcagttg agttccctag gggagaaaag cacgcttaag aattgacata aagagtagga |
| | | agttagctaa tgcaacatat atcactttgt tttttcacaa ctacagtgac tttatgtatt |
| | | tcccagagga aggcatacag ggaagaaatt atcccatttg gacaaacagc atgttctcac |
| | | aggaagcatt tatcacactt acttgtcaac tttctagaat caaatctagt agctgacagt |
| | | accaggatca ggggtgccaa ccctaagcac ccccagaaag ctgactggcc ctgtggttcc |
| | | cactccagac atgatgtcag ctgtgaaatc gacgtcgctg gaccataatt aggcttctgt |
| | | tcttcaggag acatttgttc aaagtcattt gggcaaccat attctgaaaa cagcccagcc |
| | | agggtgatgg atcactttgc aaagatcctc aatgagctat tttcaagtga tgacaaagtg |
| | | tgaagttaac cgctcatttg agaactttct ttttcatcca aagtaaattc aaatatgatt |
| | | agaaatctga ccttttatta ctggaattct cttgactaaa agtaaaattg aattttaatt |
| | | cctaaatctc catgtgtata cagtactgtg ggaacatcac agattttggc tccatgccct |
| | | aaagagaaat tggctttcag attatttgga ttaaaaacaa agactttctt aagagatgta |
| | | aaattttcat gatgttttct tttttgctaa aactaaagaa ttattctttt acatttca |
| 112 | Modified | ggtttgtttc cttttttaaa atacattgag tatgcttgcc ttttagatat agaaatatct |
| | FIX | gatgctgtct tcttcactaa attttgatta catgatttga cagcaatatt gaagagtcta |
| | intron | acagccagca cgcaggttgg taagtactgg ttctttgtta gctaggtttt cttcttcttc |
| | | atttttaaaa ctaaatagat cgacattgct tttgttgcat ttatgtttaa taaacactgt |
| | | tcagttcatg atttggtcat gtaattcctg ttagaaaaca ttcatctcct tggtttaaaa |
| | | aaattaaaag tgggaaaaca aagaaatagc agaatatagt gaaaaaaaat aaccacatta |
| | | tttttgtttg gacttaccac tttgaaatca aattgggaaa caaaagcaca aacaatggcc |
| | | ttatttacac aaaaagtctg attttaagat atatgacatt tcaaggtttc agaagtatgt |
| | | aatgaggtgt gtctctaatt ttttaaatta tatatcttca atttaaagtt ttagttaaaa |
| | | cataaagatt aacctttcat tagcaagctg ttagttatca ccaacgcttt tcatggatta |
| | | ggaaaaaatc attttgtctc tttgtcaaac atcttggagt tgatatttgg ggaaacacaa |
| | | tactcagttg agttccctag gggagaaaag cacgcttaag aattgacata aagagtagga |
| | | agttagctat tgcaacatat atcactttgt tttttcacaa ctacagtgac tttttgtatt |
| | | tcccagagga aggcatacag ggaagaaatt atcccatttg gacaaacagc ttgttctcac |
| | | aggaagcatt tatcacactt acttgtcaac tttctagaat caaatctagt agctgacagt |
| | | accaggatca ggggtgccaa ccctaagcac ccccagaaag ctgactggcc ctgtggttcc |
| | | cactccagac atgatgtcag ctgtgaaatc gacgtcgctg gaccataatt aggcttctgt |
| | | tcttcaggag acatttgttc aaagtcattt gggcaaccat attctgaaaa cagcccagcc |
| | | agggtgttgg atcactttgc aaagatcctc attgagctat tttcaagtgt tgacaaagtg |
| | | tgaagttaac cgctcatttg agaactttct ttttcatcca aagtaaattc aaatatgatt |
| | | agaaatctga ccttttatta ctggaattct cttgactaaa agtaaaattg aattttaatt |
| | | cctaaatctc catgtgtata cagtactgtg ggaacatcac agattttggc tccatgccct |
| | | aaagagaaat tggctttcag attatttgga ttaaaaacaa agactttctt aagagatgta |
| | | aaattttctt gttgttttct tttttgctaa aactaaagaa ttattctttt acatttca |
| 113 | chicken | gcgggagtcg ctgcgttgcc ttcgccccgt gccccgctcc gccgccgcct cgcgccgccc |
| | beta- | gccccggctc tgactgaccg cgttactccc acaggtgagc gggcgggacg gcccttctcc |
| | globin | tccgggctgt aattagcgct tggtttaatg acggcttgtt tcttttctgt ggctgcgtga |
| | intron | aagccttgag gggctccggg agggcccttt gtgcgggggg agcggctcgg ggggtgcgtg |
| | | cgtgtgtgtg tgcgtgggga gcgccgcgtg cggctccgcg ctgcccggcg gctgtgagcg |
| | | ctgcgggcgc ggcgcggggc tttgtgcgct ccgcagtgtg cgcgagggga gcgcggccgg |
| | | gggcggtgcc ccgcggtgcg gggggggctg cgaggggaac aaaggctgcg tgcggggtgt |
| | | gtgcgtgggg gggtgagcag ggggtgtggg cgcgtcggtc gggctgcaac cccccctgca |
| | | cccccctccc cgagttgctg agcacggccc ggcttcgggt gcggggctcc gtacggggcg |
| | | tggcgcgggg ctcgccgtgc cgggcggggg gtggcggcag gtgggggtgc cgggcggggc |
| | | ggggccgcct cgggccgggg agggctcggg ggaggggcgc ggcggccccc ggagcgccgg |
| | | cggctgtcga ggcgcggcga gccgcagcca ttgcctttta tggtaatcgt gcgagagggc |
| | | gcagggactt cctttgtccc aaatctgtgc ggagccgaaa tctgggaggc gccgccgcac |
| | | cccctctagc gggcgcgggg cgaagcggtg cggcgccggc aggaaggaaa tgggcgggga |
| | | gggccttcgt gcgtcgccgc gccgccgtcc ccttctccct c |
| 114 | Modified | gcgggagtcg ctgcgttgcc ttcgccccgt gccccgctcc gccgccgcct cgcgccgccc |
| | chicken | gccccggctc tgactgaccg cgttactccc acaggtgagc gggcgggacg gcccttctcc |
| | beta- | tccgggctgt aattagcgct tggtttaatg acggcttgtt tcttttctgt ggctgcgtga |
| | globin | aagccttgag gggctccggg agggcccttt gtgcgggggg agcggctcgg ggggtgcgtg |
| | intron | cgtgtgtgtg tgcgtgggga gcgccgcgtg cggctccgcg ctgcccggcg gctgtgagcg |
| | | ctgcgggcgc ggcgcggggc tttgtgcgct ccgcagtgtg cgcgagggga gcgcggccgg |
| | | gggcggtgcc ccgcggtgcg gggggggctg cgaggggaac aaaggctgcg tgcggggtgt |
| | | gtgcgtgggg gggtgagcag ggggtgtggg cgcgtcggtc gggctgcaac cccccctgca |
| | | cccccctccc cgagttgctg agcacggccc ggcttcgggt gcggggctcc gtacggggcg |
| | | tggcgcgggg ctcgccgtgc cgggcggggg gtggcggcag gtgggggtgc cgggcggggc |
| | | ggggccgcct cgggccgggg agggctcggg ggaggggcgc ggcggccccc ggagcgccgg |
| | | cggctgtcga ggcgcggcga gccgcagcca ttgccttttt tggtaatcgt gcgagagggc |
| | | gcagggactt cctttgtccc aaatctgtgc ggagccgaaa tctgggaggc gccgccgcac |
| | | cccctctagc gggcgcgggg cgaagcggtg cggcgccggc aggaaggaat tgggcgggga |
| | | gggccttcgt gcgtcgccgc gccgccgtcc ccttctccct c |
| 115 | bgH poly | tgtgccttctagttgccagccatctgttgtttgcccctcccccgtgccttccttgaccctggaaggtgcca |
| | A | ctcccactgtcctttcctaataaaatgaggaaattgcatcgcattgtctgagtaggtgtcattctattctg |
| | | gggggtggggtgggggctagctctaga |
| 116 | hFIX | atgcagcgcg tgaacatgat catggcagaa tcaccaggcc tcatcaccat ctgcctttta |
| | gene | ggatatctac tcagtgctga atgtacaggt ttgtttcctt ttttaaaata cattgagtat |
| | | gcttgccttt tagatataga aatatctgat gctgtcttct tcactaaatt ttgattacat |
| | | gatttgacag caatattgaa gagtctaaca gccagcacgc aggttggtaa gtactggttc |
| | | tttgttagct aggttttctt cttcttcatt tttaaaacta aatagatcga caatgcttat |
| | | gatgcattta tgtttaataa acactgttca gttcatgatt tggtcatgta attcctgtta |
| | | gaaaacattc atctccttgg tttaaaaaaa ttaaaagtgg gaaaacaaag aaatagcaga |
| | | atatagtgaa aaaaaataac cacattattt ttgtttggac ttaccacttt gaaatcaaaa |
| | | tgggaaacaa aagcacaaac aatggcctta tttacacaaa aagtctgatt ttaagatata |
| | | tgacatttca aggtttcaga agtatgtaat gaggtgtgtc tctaattttt taaattatat |
| | | atcttcaatt taaagtttta gttaaaacat aaagattaac ctttcattag caagctgtta |
| | | gttatcacca aagcttttca tggattagga aaaaatcatt ttgtctctat gtcaaacatc |
| | | ttggagttga tatttgggga aacacaatac tcagttgagt tccctagggg agaaaagcaa |
| | | gcttaagaat tgacataaag agtaggaagt tagctaatgc aacatatatc actttgtttt |
| | | ttcacaacta cagtgacttt atgtatttcc cagaggaagg catacaggga agaaattatc |
| | | ccatttggac aaacagcatg ttctcacagg aagcatttat cacacttact tgtcaacttt |
| | | ctagaatcaa atctagtagc tgacagtacc aggatcaggg gtgccaaccc taagcacccc |
| | | cagaaagctg actggccctg tggttcccac tccagacatg atgtcagctg tgaaatcgac |
| | | gtcgctggac cataattagg cttctgttct tcaggagaca tttgttcaaa gtcatttggg |
| | | caaccatatt ctgaaaacag cccagccagg gtgatggatc actttgcaaa gatcctcaat |
| | | gagctatttt caagtgatga caaagtgtga agttaaccgc tcatttgaga actttctttt |
| | | tcatccaaag taaattcaaa tatgattaga aatctgacct tttattactg gaattctctt |
| | | gactaaaagt aaaattgaat tttaattcct aaatctccat gtgtatacag tactgtggga |
| | | acatcacaga ttttggctcc atgccctaaa gagaaattgg ctttcagatt atttggatta |
| | | aaaacaaaga ctttcttaag agatgtaaaa ttttcatgat gttttctttt ttgctaaaac |
| | | taaagaatta ttcttttaca tttcagtttt tcttgatcat gaaaacgcca acaaaattct |
| | | gaatcggcca aagaggtata attcaggtaa attggaagag tttgttcaag ggaaccttga |
| | | gagagaatgt atggaagaaa agtgtagttt tgaagaagca cgagaagttt ttgaaaacac |
| | | tgaaagaaca actgaatttt ggaagcagta tgttgatgga gatcagtgtg agtccaatcc |
| | | atgtttaaat ggcggcagtt gcaaggatga cattaattcc tatgaatgtt ggtgtccctt |
| | | tggatttgaa ggaaagaact gtgaattaga tgtaacatgt aacattaaga atggcagatg |
| | | cgagcagttt tgtaaaaata gtgctgataa caaggtggtt tgctcctgta ctgagggata |
| | | tcgacttgca gaaaaccaga agtcctgtga accagcagtg ccatttccat gtggaagagt |
| | | ttctgtttca caaacttcta agctcacccg tgctgaggct gtttttcctg atgtggacta |
| | | tgtaaattct actgaagctg aaaccatttt ggataacatc actcaaagca cccaatcatt |
| | | taatgacttc actcgggttg ttggtggaga agatgccaaa ccaggtcaat tcccttggca |
| | | ggttgttttg aatggtaaag ttgatgcatt ctgtggaggc tctatcgtta atgaaaaatg |
| | | gattgtaact gctgcccact gtgttgaaac tggtgttaaa attacagttg tcgcaggtga |
| | | acataatatt gaggagacag aacatacaga gcaaaagcga aatgtgattc gaattattcc |
| | | tcaccacaac tacaatgcag ctattaataa gtacaaccat gacattgccc ttctggaact |
| | | ggacgaaccc ttagtgctaa acagctacgt tacacctatt tgcattgctg acaaggaata |
| | | cacgaacatc ttcctcaaat ttggatctgg ctatgtaagt ggctggggaa gagtcttcca |
| | | caaagggaga tcagctttag ttcttcagta ccttagagtt ccacttgttg accgagccac |
| | | atgtcttcga tctacaaagt tcaccatcta taacaacatg ttctgtgctg gcttccatga |
| | | aggaggtaga gattcatgtc aaggagatag tgggggaccc catgttactg aagtggaagg |
| | | gaccagtttc ttaactggaa ttattagctg gggtgaagag tgtgcaatga aaggcaaata |
| | | tggaatatat accaaggtat cccggtatgt caactggatt aaggaaaaaa caaagctcac |
| | | ttaa |
| 117 | Synthetic | ccctaaaatg ggcaaacatt gcaagcagca aacagcaaac acacagccct ccctgcctgc |
| | promoter | tgaccttgga gctggggcag aggtcagaga cctctctggg cccatgccac ctccaacatc |
| | LP1 | cactcgaccc cttggaattt cggtggagag gagcagaggt tgtcctggcg tggtttaggt |
| | | agtgtgagag gggaatgact cctttcggta agtgcagtgg aagctgtaca ctgcccaggc |
| | | aaagcgtccg ggcagcgtag gcgggcgact cagatcccag ccagtggact tagcccctgt |
| | | ttgctcctcc gataactggg gtgaccttgg ttaatattca ccagcagcct cccccgttgc |
| | | ccctctggat ccactgctta aatacggacg aggacagggc cctgtctcct cagcttcagg |
| | | caccaccact gacctgggac agtgaatccg gactctaagg taaatataaa atttttaagt |
| | | gtataatgtg ttaaactact gattctaatt gtttctctct tttagattcc aacctttgga |
| | | actgaattct agaccacc |
| 118 | Synthetic | TGCATGTATAATTTCTACAGAACCTATTAGAAAGGATCACCCAGCCTCTGCTTTTGTACAACTTTCCCTTA |
| | promoter | AAAAACTGCCAATTCCACTGCTGTTTGGCCCAATAGTGAGAACTTTTTCCTGCTGCCTCTTGGTGCTTTTG |
| | TBG+TSS | CCTATGGCCCCTATTCTGCCTGCTGAAGACACTCTTGCCAGCATGGACTTAAACCCCTCCAGCTCTGACAA |
| | | TCCTCTTTCTCTTTTGTTTTACATGAAGGGTCTGGCAGCCAAAGCAATCACTCAAAGTTCAAACCTTATCA |
| | | TTTTTTGCTTTGTTCCTCTTGGCCTTGGTTTTGTACATCAGCTTTGAAAATACCATCCCAGGGTTAATGCT |
| | | GGGGTTAATTTATAACTAAGAGTGCTCTAGTTTTGCAATACAGGACATGCTATAAAAATGGAAAGATGTTG |
| | | CTTTCTGAGAG |
| 119 | Synthetic | TTAAGAATATTTCATAGAACGAATGTTCCGATGCTCTAATCTCTCTAGACAAGGTTCATATTTGTATGGGT |
| | promoter | TACTTATTCTCTCTTTGTTGACTAAGTCAATAATCAGAATCAGCAGGTTTGCAGTCAGATTGGCAGGGATA |
| | TTR | AGCAGCCTAGCTCAGGAGAAGTGAGTATAAAAGCCCCAGGCTGGGAGCAGCCATCACA |
| 120 | Synthetic | TTAAGAATATTTCATAGAACGAATGTTCCGATGCTCTAATCTCTCTAGACAAGGTTCATATTTGTATGGGT |
| | promoter | TACTTATTCTCTCTTTGTTGACTAAGTCAATAATCAGAATCAGCAGGTTTGCAGTCAGATTGGCAGGGATA |
| | HSE | AGCAGCCTAGCTCAGGAGAAGTGAGTATAAAAGCCCCAGGCTGGGAGCAGCCATCACA |
| 121 | Synthetic | taaagcaaat atttgtggtt atggattaac tcgaacttct agaagctgtt tgcccactct |
| | promoter | atttgcccat cctaggtagg cgccctttgg accttttgca atcctggctt ctagaagagc |
| | SERPIN | aaacagcaaa cacatcctag gtaggactta gcccctgttt gctcctccga taactggggt |
| | A1_COMP | gaccttggtt aatattcacc agcagcctca tgctagcctc gaggatatca gatctgggcg |
| | | actcagatcc cagccagtgg acttagcccc tgtttgctcc tccgataact ggggtgacct |
| | | tggttaatat tcaccagcag cctcccccgt tgcccctctg gatccactgc ttaaatacgg |
| | | acgaggacag ggccctgtct cctcagcttc aggcaccacc actgacctgg gacagtgaat |
| | | cgccacc |
| 122 | Heat- | GTCGAGAACGTTCTAGAACGTCG |
| | shock | |
| | element | |

1. A hybrid adeno-associated virus (AAV) vector, comprising a recombinant hybrid AAV capsid protein and a transgene of interest under the control of an hepatocyte-specific promoter,
wherein the capsid protein is a hybrid between at least two recombinant AAV capsid proteins from different AAV serotypes, an acceptor AAV capsid protein and at least one donor AAV capsid protein,
said hybrid AAV capsid protein comprising at least one hypervariable region (HVR) sequence from the donor AAV capsid protein(s), selected among the HVR1, HVR5, HVR7 or HVR10 sequence, replacing the corresponding HVR sequence(s) of the acceptor AAV capsid protein.

2. The hybrid AAV vector according to claim 1, wherein the donor AAV capsid serotype is AAV2, AAV13 or hybrid AAV2/13; preferably the hybrid AAV2/13 capsid is selected from the group consisting of the sequences SEQ ID NO: 2 to 30; and/or wherein the acceptor AAV capsid serotype is selected from the group consisting of: AAV8, AAV9, AAV5, AAV-LK03, AAVrh74, AAV9.rh74, and AAVrh10.

3. The hybrid AAV vector according to claim 1 or claim 2, wherein the HVR1 sequence from the donor AAV capsid protein consists of:
EX₁X₂KTAPGKKRX₃VX₄X₅X₆X₇X₈EPDSSSGX₉GKX₁₀GX₁₁,
wherein X₁ is P, H, A; X₂ is V or A; X₃ is P or A; X₄ is E or A; X₅ is H or Q; X₆ is S or A; X₇ is P or H; X₈ is V or A; X₉ is I or T; X₁₀ is A or S; and X₁₁ is Q, K or N.

4. The hybrid AAV vector according to claim 1, wherein the HVR5 sequence from the donor AAV capsid protein consists of:
YYLX₁X₂TX₃X₄X₅SGTX₆X₇X₈SRLX₉FSQAGX₁₀X₁₁X₁₂X₁₃X₁₄X₁₅X₁₆
X₁₇X₁₈NWLPGP,
wherein X₁ is N or S; X₂ is K or R; X₃ is Q or N; X₄ is T, S or A; X₅ is P, T, N or A; X₆ is L, P, T, V or is absent; X₇ is Q or T; X₈ is Q or M; X₉ is Q or L; X₁₀ is P or A; X₁₁ is T or S; X₁₂ is S or D; X₁₃ is M or I; X₁₄ is S or R; X₁₅ is L or D; X₁₆ is Q or H; X₁₇ is A or S; and X₁₈ is K or R.

5. The hybrid AAV vector according to claim 1, wherein the HVR7 sequence from the donor AAV capsid protein consists of:
SX₁X₂X₃WTX₄ATKYH,
wherein X₁ is D, E or N; X₂ is Y or F; X₃ is S or P; X₄ is G or A.

6. The hybrid AAV vector according to claim 1, wherein the HVR10 sequence from the donor AAV capsid protein consists of:
TEQYGX₁VSX₂NLQX₃X₄NX₅X₆X₇X₈TX₉X₁₀VNX₁₁QGX₁₂LX₁₃GMV
WQNRD,
wherein X₁ is S, T, A, Y or N; X₂ is T or N; X₃ is R, N or S; X₄ is G or S; X₅ is R, A or T; X₆ is Q, G, R or A; X₇ is A or P; X₈ is A, T or S; X₉ is A, S, G or E; X₁₀ is T, D or N; X₁₁ is A, T, H or N; X₁₂ is A, I or V; and X₁₃ is P or S.

7. The hybrid AAV vector according to claim 1, wherein the recombinant hybrid AAV capsid protein comprises or consists of a sequence selected from the group consisting of the sequences SEQ ID NO: SEQ ID NO: 65, 68, 71, 74, 77, 80, 83, 86, 89, 95, 101 and the sequences having at least 85%, 90%, 95%, 97%, 98% or 99% identity with said sequences.

8. The hybrid AAV vector according to claim 1, wherein the hepatocyte-specific promoter is selected from the group comprising: an alpha-1 antitrypsin (AAT) promoter, a transthyretin (TTR) promoter, an albumin (ALB) promoter, a thyroxine-binding globulin (TBG) promoter hepatitis B virus (HBV) core promoter human alpha-1 antitrypsin (hAAT) promoter of SEQ ID NO: 107, LP1 promoter of SEQ ID NO: 117, HLP promoter, SERPINA1_COMP_D promoter of SEQ ID NO: 121, TTR promoter of SEQ ID NO: 119, HSE promoter of SEQ ID NO: 120, TBG full-length promoter, TBG minimal promoter, and TBG-derived promoter of SEQ ID NO: 118.

9. The hybrid AAV vector according to claim 1, wherein the hepatocyte-specific promoter is associated to an enhancer sequence capable of enhancing liver-specific expression of genes.

10. The hybrid AAV vector according to claim 1, wherein the transgene expression cassette comprises, in the 5′ to 3′ orientation, an hepatocyte-specific promoter, the transgene coding sequence; and a polyadenylation signal such as (bGH) polyA.

11. The hybrid AAV vector according to claim 1, wherein the transgene of interest is selected from the group consisting of: therapeutic genes; genes encoding therapeutic proteins or peptides.

12. The hybrid AAV vector according to claim 1, wherein the transgene encodes a protein with a therapeutic effect on hepatocytes or a therapeutic protein that is secreted from the liver into the blood-stream.

13. A pharmaceutical composition comprising a therapeutically effective amount of hybrid AAV vector according to claim 1.

14-15. (canceled)

16. A method for treating a disease by expression of a therapeutic gene in the liver comprising administering to a patient a therapeutically effective amount of the hybrid AAV vector according to claim 1.

17. A method for treating a disease according to claim 16, wherein said disease is a genetic disease selected from the group consisting of: Hemophilia A, Hemophilia B, X-linked hypophosphatemia, Crigler-Najjar syndrome, Glucose-6-phosphate dehydrogenase deficiency and Glycogen storage diseases types I, II and III.

18. The hybrid AAV vector according to claim 1, wherein the HVR1 sequence from the donor AAV capsid is selected from the group consisting of SEQ ID NO: 33 to 40.

19. The hybrid AAV vector according to claim 1, wherein the HVR5 sequence from the donor AAV capsid is selected from the group consisting of SEQ ID NO: 41 to 49.

20. The hybrid AAV vector according to claim 1, wherein the HVR7 sequence from the donor AAV capsid is selected from the group consisting of SEQ ID NO: 50 to 55.

21. The hybrid AAV vector according to claim 1, wherein the HVR10 sequence from the donor AAV capsid is selected from the group consisting of SEQ ID NO: 56 to 64.

22. The hybrid AAV vector according to claim 12, wherein said protein is selected from the group consisting of: Acid Alpha-Glucosidase (GAA), FGF23 antagonist, Amylo-Alpha-1,6-Glucosidase, 4-Alpha-Glucanotransferase (AGL), Glucose-6-phosphate dehydrogenase (G6PD) and Uridine Diphosphate Glucuronosyltransferase isoform 1A1 (UGT1A1).


SEQUENCE LISTING (122 sequences)

SEQ ID NO: 1 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 2 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 3 - protein, 735 aa, synthetic construct
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLNRTQTXSGTPTQSRLLFSQAGPTSMSLQAKNWLPGPCYRQQRLSKQANDNNNSNFPWTGATKYHLNGRDSLVNPGPAMASHKDDEEKFFPMHGTLIFGKQGSEKTNAELENVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEARPIGTRYLTRNL

SEQ ID NO: 4 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEERVKTAPGKKRPVAVAHQWQTITRVPMEWVIPQEKRLNFGQTGDADWHCDPQPLGQPPAAPSGLGSTTMATGSGALLLLESLSADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTTTMSRLLFSQAGPTSMSLHAKNWLPGPCYRQQRLSKQANDNNNSNFPWTAATKYHLNGSDSLINPGPAMASHKDDEEKFFPQSGVLIFGKQGSEKTNVDIEKVMITDEEEIRTTNPVATEQYGSVSTNLQRGNRRAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 5 - protein, 735 aa, synthetic construct
MAADGYLPDWLEDTLSEGIRQWWKLKPGPPPPKSAERHKDDSRGLVLPGYKYLGPFXGLDKGEPVNEADAAALEHDKAYDRQLDSGDNPYLKYNHADAEFQERLKEDTSFGGNLGRAVFQAKKRVLXPLGXVEEPVKTAPGKKRPVEHSPVEPDSSSGTGKAGQQPARKRLNFGQTGDSDSSSRDMRKVFSAAAPTSLGSTTMASGGGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQTNSGTLQQSRLLFSQAGPTNMSLQAKNWLPGPCYRQQRLSKQANDNNNSNFPWTAATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKEXSKRWNPEIQYTSNYNKSVNVDFTVDTNGVYSEPRPIGTRYLTRNL

SEQ ID NO: 6 - protein, 735 aa, synthetic construct
MAADGYLPDWLGETLSQGIRQWWKLKPGPPPPKPAERHKXDSRGLVLPGYKYLGPFNGLDKGEPVNEADAAALEHDKAYDRQLDSGHIPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTTTMATGSGAPVADNNEGADGVGNSSGNWHCDSQWLGDRVITTSTRTWALPTYNNHLYNFFQIKINASNDNHYFGYSTPWGYFDFNRFHCHFSPRDWQRLINNNWGFRPKRLNFKLFLIQVKEVTQNDGTTTIANNLTSTVQVFTXSEYQLPYVLGSAHQGCXPPFPADVFMVPQYGYLTLNNGSQAXGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGYVSNNLQNSNRQAATADVNTQGVLPGMVWQDRDVYLQGPIWAKIPHTDGHFHPSPLMGGFGLKHPPPQILIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 7 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMEDNNEGADGVGNSSGNWHCDSQWLGDRVITTSTRTWALPTYNNHLYKQICSQSGASNDNHYFGYSTRWGDFDFNRFHCHFSPRDWQRLINNNWGFRPKRLNFKLFNIQVKEVTQNDGTXTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLLFSQAGPTSMSLQAKNWLPGPCYRQQRLSKQANDNNNSNFPWTAATKYHLNGRDSLVNPGPAMASHKDDEEKFFPQSGVLIFGKQGSEKTNVDIEKVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKSSXVPANPPTNFSSAKFASFITQYSTGQVSVEIEWELQKENSKRWNPEIQYTSNYNKSVNVDFTVDTNGVYSEPRPIGTRYLTRNL

SEQ ID NO: 8 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLNKTQSSGTLQQSRLLFSQAGPTSMSLQARNWLPGPCYRQQRVSKTSADKNNSEYSWTAATKYHLNGRDSLVNPGPAMASHKDDEEKFFPMPGTLIFGKEGTNANNAELENVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 9 - protein, 734 aa, synthetic construct
MAADGYLPDWLEDTLSEGIRQWWKLKPGPPPPKPAERHKDDSRGLVLPGYKYLGPFNGLDKGEPVNEADAAAIEHDKAYDRQLDSGDNPYLKYNHADAEFQERLKEDTSFGGNLGRAVFQAKKRVLEXXGLVEEPVKTAPGKKRPVAHSPAEPDSSSGTGKAGNQPARKRLNFGQTGDADSVPDPQPLGQPPAAPTSLGSTTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNYSWTGATKYHLNGRDSLVXPGPAMASHXDDEXKFFPMHGTLIFGKEGTNATNADLDNVMITDEEEIRTTNPVATEQYGYVSNNLQNSNTAATTETVNHQGALPGMVWQDRDVYLQGPIWAKIPHTDGHFHPSPLMGGFGLKNPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 10 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLARAVFQAKKRVLEPLGLVEEPVKTAPGKKRPVEHSPAEPDSSSGTGKSGNQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLNKTQTNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLENVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 11 - protein, 736 aa, synthetic construct
MAADGYLPDWLQENLFFDIRQWWKLKPGPPPPKPAERHQDNSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGKQPAXKRLNFGQTGXADSVPDPQPLGQPPAAPSGLGTNTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNKAGKRQQQQQLSLDCGYKVSTKWPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 12 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQSGNTQAATGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 13 - protein, 735 aa, synthetic construct
MAADGYLPDWLEDTLSEGIRQWWKLKPGPPPPKPAERHKDDNRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 14 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 15 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLENVMITDEEEIRTTNPVATEQYGTVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 16 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGKPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSSKFASFITQYSTGQVSVEIEWELQKENSKRWNPEIQYTSNYNKSVNVDFTVDTNGVYSEPRPIGTRYLTRNL

SEQ ID NO: 17 - protein, 735 aa, synthetic construct; identical to SEQ ID NO: 14

SEQ ID NO: 18 - protein, 736 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQANSGTLQQSRLLFSQAGPTSMSLQAKNWLPGPCYRQQRLSKQANDNNNSNFPWTAATKYHLNGRDSLVNPGPAMASHKDDEEEKFFPMHGTLIFGKQGTNANDADLDNVMITDEEEIRTTNPVATEQYXYVSNNLQNSNTGPTTGTVNHQGALPGMVWQDRDVYLQGPIWAKIPHTDGHFHPSPLMGGFGLKHPPPQIMIKNTPVPSPLMGGFGLKHPPPQITQYSTGQVSVEIEWELQKENSKRWNPEIQYTSNYNKSVNVDFTVDTNGVYSEPRPIGTRYLTRNL

SEQ ID NO: 19 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGIGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 20 - protein, 735 aa, synthetic construct
MAADGYLPDWLEDTLSEGIRQWWKLKPGPPPPKPAERHKDNSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPAKKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTNMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 21 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGNQPARKRLNFGQTGDADSVPDPQPIGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTVQQSRLLFSQAGPTSMSLQAKNWLPGPCYRQQRLSKQASDNNNSNFPWTAATKYHLNGRDSLVNPGPAMASHKDDEEKFFPMHGTLIFGKEGTTSNNADLEHVMITDEEEIRTTNPVATEQYGNVSNNLQNSNTGPTTENVNNQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 22 - protein, 734 aa, synthetic construct
MAADGYLPDWLEDNLSEGIREWWALKPGAPQPKANQQHQDNARGLVLPGYKYLGPGNGLDKGEPVNAADAAALEHDKAYDKQLKAGDNPYLKYNHADAEFQERLKEDTSFGGNLGRAVFQAKKRVLEPLGLVEEPVKTAPGKKRPVEHSPAEPDSSSGTGKSGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTAATKYHLNGRDSLVNPGPAMASHKDDEEKFFPMHGTLIFGKEGTNATNADLDNVMITDEEEIRTTNPVATEQYGYVSNNLQNSNTAATTETVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 23 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGNQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 24 - protein, 735 aa, synthetic construct; identical to SEQ ID NO: 14

SEQ ID NO: 25 - protein, 735 aa, synthetic construct; identical to SEQ ID NO: 14

SEQ ID NO: 26 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 27 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGSTTMATGSGAPMADNNEGADGVGNSSGNWHCDSQWLGNRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 28 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTGRDNADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 29 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLETLGLVEEHVKTAPGKKRAVEHAHVEPDSSSGTGKAGQQPARKRLNFGQTGDADSVPDPQPLGQPPAAPXGLGSTTMASGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQATDNNNSEFPWTAATKYHLNGRDSLVNPGPAMASHKDDEEKFFPMHGTLIFGKQGTKCNDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 30 - protein, 736 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGNQPARKRLNFGQTGDADSVPDPQPLGEPPAAPTSLGSTTMASGGGAPVADNNEGADGVGNSSGKLALRFPMAGRQSHYHQHPNLGPAHLQQPSVQANFQPIRSQQRQPLILAHCNPWGYFDFNRFHCHFSPRDWQRLINNNWGFRPKRLNFKLFNIQVKEVTQNDGTTTIANNLTSTVQVFTDSEYQIPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSASGTVQQSRLLFSQAGPTSMSLQAKNWLPGPCYRQQRLSKQANENNNSNFPWTAATKYHLNGRDSLVNPGPAMASHKDDEEKFFPMHGTLIFGKQGTGRDNADLDNVMITDEEEIRTTNPVATEQYGYVSNNLQNSNTAPSTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 31 - protein, 736 aa, synthetic construct
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 32 - protein, 733 aa, synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 33 - protein, 31 aa, synthetic construct
EPVKTAPGKKRPVEHSPVEPDSSSGTGKAGQ

SEQ ID NO: 34 - protein, 31 aa, synthetic construct
EPVKTAPGKKRPVEHSPAEPDSSSGTGKAGQ

SEQ ID NO: 35 - protein, 31 aa, synthetic construct
EPVKTAPGKKRPVAHSPAEPDSSSGTGKAGN

SEQ ID NO: 36 - protein, 31 aa, synthetic construct
EPVKTAPGKKRPVEHSPAEPDSSSGTGKSGN

SEQ ID NO: 37 - protein, 31 aa, synthetic construct
EPVKTAPGKKRPVEHSPVEPDSSSGTGKAGN

SEQ ID NO: 38 - protein, 31 aa, synthetic construct
EHVKTAPGKKRAVEHAHVEPDSSSGTGKAGQ

SEQ ID NO: 39 - protein, 31 aa, synthetic construct
EAAKTAPGKKRPVEQSPAEPDSSSGIGKSGQ

SEQ ID NO: 40 - protein, 31 aa, synthetic construct
EPVKTAPGKKRPVEHSPAEPDSSSGTGKSGK

SEQ ID NO: 41 - protein, 39 aa, synthetic construct
YYLSRTNTPSGTTTQSRLLFSQAGPTSMSLQAKNWLPGP

SEQ ID NO: 42 - protein, 39 aa, synthetic construct
YYLNKTQSNSGTTTMSRLLFSQAGPTSMSLHAKNWLPGP

SEQ ID NO: 43 - protein, 39 aa, synthetic construct
YYLNRTQTTSGTPTQSRLLFSQAGPTSMSLQAKNWLPGP

SEQ ID NO: 44 - protein, 38 aa, synthetic construct
YYLNRTQTASGTQQSRLLFSQAGPTSMSLQAKNWLPGP

SEQ ID NO: 45 - protein, 39 aa, synthetic construct
YYLNKTQSNSGTVQQSRLLFSQAGPTSMSLQAKNWLPGP

SEQ ID NO: 46 - protein, 39 aa, synthetic construct
YYLNKTQSASGTVQQSRLLFSQAGPTSMSLQAKNWLPGP

SEQ ID NO: 47 - protein, 39 aa, synthetic construct
YYLNKTQANSGTLQQSRLLFSQAGPTSMSLQAKNWLPGP

SEQ ID NO: 48 - protein, 39 aa, synthetic construct
YYLNKTQTNSGTLQQSRLLFSQAGPTSMSLQAKNWLPGP

SEQ ID NO: 49 - protein, 39 aa, synthetic construct
YYLSRTNTPSGTTTQSRLQFSQAGASDIRDQSRNWLPGP

SEQ ID NO: 50 - protein, 12 aa, synthetic construct
SEYSWTGATKYH

SEQ ID NO: 51 - protein, 12 aa, synthetic construct
SNFPWTGATKYH

SEQ ID NO: 52 - protein, 12 aa, synthetic construct
SNFPWTAATKYH

SEQ ID NO: 53 - protein, 12 aa, synthetic construct
SEYSWTAATKYH

SEQ ID NO: 54 - protein, 12 aa, synthetic construct
SNYSWTGATKYH

SEQ ID NO: 55 - protein, 12 aa, synthetic construct
SEFPWTAATKYH

SEQ ID NO: 56 - protein, 38 aa, synthetic construct
TEQYGSVSTNLQRGNRQAATADVNTQGVLPGMVWQDRD

SEQ ID NO: 57 - protein, 38 aa, synthetic construct
TEQYGTVSNNLQNSNAGPTTGTVNHQGALPGMVWQDRD

SEQ ID NO: 58 - protein, 38 aa, synthetic construct
TEQYGYVSNNLQNSNTGPTTGTVNHQGALPGMVWQDRD

SEQ ID NO: 59 - protein, 38 aa, synthetic construct
TEQYGSVSTNLQRGNRRAATADVNTQGVLPGMVWQDRD

SEQ ID NO: 60 - protein, 38 aa, synthetic construct
TEQYGYVSNNLQNSNRQAATADVNTQGVLPGMVWQDRD

SEQ ID NO: 61 - protein, 38 aa, synthetic construct
TEQYGYVSNNLQNSNTAATTETVNHQGALPGMVWQDRD

SEQ ID NO: 62 - protein, 38 aa, synthetic construct
TEQYGYVSNNLQSGNTQAATGTVNHQGALPGMVWQDRD

SEQ ID NO: 63 - protein, 38 aa, synthetic construct
TEQYGNVSNNLQNSNTGPTTENVNNQGALPGMVWQDRD

SEQ ID NO: 64 - protein, 38 aa, synthetic construct
TEQYGYVSNNLQNSNTAPSTGTVNHQGALPGMVWQDRD

SEQ ID NO: 65 - protein, 737 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLNRTQTASGTQQSRLLFSQAGPTSMSLQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 66 - DNA, 2214 bp, synthetic construct
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtattatctaaacaggacacaaacagccagtggaactcagcagtctcggctactgtttagccaagctggacccaccagcatgtctcttcaagctaaaaactggctgcctggaccttgctaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 67 - DNA, 114 bp, synthetic construct
  source 1..114: mol_type = other DNA
tattatctaaacaggacacaaacagccagtggaactcagcagtctcggctactgtttagccaagctggacccaccagcatgtctcttcaagctaaaaactggctgcctggacct

SEQ ID NO: 68 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLNKTQSNSGTVQQSRLLFSQAGPTSMSLQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 69 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtattatctgaacaagacacaatcaaatagtggaactgttcagcagtctcggctactgtttagccaagctggacccaccagcatgtctcttcaagctaaaaactggctgcctggaccttgctaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 70 - DNA, 117 bp, synthetic construct
  source 1..117: mol_type = other DNA
tattatctgaacaagacacaatcaaatagtggaactgttcagcagtctcggctactgtttagccaagctggacccaccagcatgtctcttcaagctaaaaactggctgcctggacct

SEQ ID NO: 71 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLNKTQSASGTVQQSRLLFSQAGPTSMSLQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 72 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtattatctgaacaagacacaatcagccagtggaactgttcagcagtctcggctactgtttagccaagctggacccaccagcatgtctcttcaagctaaaaactggctgcctggaccttgctaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 73 - DNA, 117 bp, synthetic construct
  source 1..117: mol_type = other DNA
tattatctgaacaagacacaatcagccagtggaactgttcagcagtctcggctactgtttagccaagctggacccaccagcatgtctcttcaagctaaaaactggctgcctggacct

SEQ ID NO: 74 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLNKTQANSGTLQQSRLLFSQAGPTSMSLQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 75 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtattatctgaacaaaacacaagcaaatagtggaactcttcagcagtctcggctactgtttagtcaagctggacccaccagcatgtctcttcaagctaaaaactggctgcctggaccttgctaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 76 - DNA, 117 bp, synthetic construct
  source 1..117: mol_type = other DNA
tattatctgaacaaaacacaagcaaatagtggaactcttcagcagtctcggctactgtttagtcaagctggacccaccagcatgtctcttcaagctaaaaactggctgcctggacct

SEQ ID NO: 77 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLNKTQSNSGTTTMSRLLFSQAGPTSMSLHAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 78 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtattatctgaacaaaacacaatcaaatagtggaaccaccacgatgtcaaggctactgtttagtcaagctggacccaccagcatgtctcttcacgctaaaaactggctgcctggaccttgctaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 79 - DNA, 117 bp, synthetic construct
  source 1..117: mol_type = other DNA
tattatctgaacaaaacacaatcaaatagtggaaccaccacgatgtcaaggctactgtttagtcaagctggacccaccagcatgtctcttcacgctaaaaactggctgcctggacct

SEQ ID NO: 80 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLNKTQTNSGTLQQSRLLFSQAGPTNMSLQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 81 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtattatctgaacaagacacaaacaaatagtggaactcttcagcagtctcggctactgtttagccaagctggaccaaccaacatgtctcttcaagctaaaaactggctgcctggaccttgctaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 82 - DNA, 117 bp, synthetic construct
  source 1..117: mol_type = other DNA
tattatctgaacaagacacaaacaaatagtggaactcttcagcagtctcggctactgtttagccaagctggaccaaccaacatgtctcttcaagctaaaaactggctgcctggacct

SEQ ID NO: 83 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTNTPSGTTTQSRLLFSQAGPTSMSLQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 84 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtattacttgagcagaacaaacactccaagtggaaccaccacgcagtctcggctactgtttagtcaagctggacccaccagcatgtctcttcaagctaaaaactggctgcctggaccttgctaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 85 - DNA, 117 bp, synthetic construct
  source 1..117: mol_type = other DNA
tattacttgagcagaacaaacactccaagtggaaccaccacgcagtctcggctactgtttagtcaagctggacccaccagcatgtctcttcaagctaaaaactggctgcctggacct

SEQ ID NO: 86 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLNRTQTTSGTPTQSRLLFSQAGPTSMSLQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 87 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtattatctaaacagaacacaaacaaccagtggaactcccacgcagtctcggctactgtttagccaagctggacccacaagcatgtctcttcaagctaaaaactggctgcctggaccgtgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 88 - DNA, 117 bp, synthetic construct
  source 1..117: mol_type = other DNA
tattatctaaacagaacacaaacaaccagtggaactcccacgcagtctcggctactgtttagccaagctggacccacaagcatgtctcttcaagctaaaaactggctgcctggaccg

SEQ ID NO: 89 - protein, 737 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEPVKTAPGKKRPVEHSPVEPDSSSGTGKAGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 90 - DNA, 2214 bp, synthetic construct
  source 1..2214: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaacctgttaagacggctccgggaaaaaagaggccggtagagcactctcctgtggagccagactcctcctcgggaaccggaaaagcgggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 91 - protein, 736 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISSQSGASNDNHYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 92 - DNA, 2211 bp, synthetic construct
  source 1..2211: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccagccaatcaggagcctcaaacgacaaccactacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 93 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRLSKQANDNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 94 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgctacagacagcagcgtctgtcaaagcaggcaaacgacaacaacaacagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 95 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFPWTAATKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 96 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttccctggactgcggctacaaagtatcatctaaatggccggaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 97 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRDSLVNPGPAMASHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 98 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggacaaagtatcatctaaatggccgggactcgttggttaatccaggaccagctatggccagccacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 99 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEEKFFPMHGTLIFGKQGTNANDADLDNVMITDEEEIRTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 100 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgatgaagaaaagtttttccccatgcatggaaccctgatatttggtaaacaaggaacaaatgctaacgacgcggatttggacaatgtcatgattacagatgaagaagaaatccgcaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 101 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEQYGYVSNNLQNSNTGPTTGTVNHQGALPGMVWQDRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 102 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacggagcagtacggatatgtgtcaaataatttgcaaaactcaaatactggtccaactactggaactgtcaatcaccaaggagcgttacctggtatggtgtggcaggatcgagacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 103 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYYKSTSVDFAVNTEGVYSEPRPIGTRYLTRNL

SEQ ID NO: 104 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacaccgatggacactttcatccttctccactgatgggaggttttgggctcaaacacccgcctcctcagatcatgatcaaaaacactcccgttccagccaatcctcctacaaacttcagttctgccaagtttgcttctttcatcacacagtattccacggggcaggtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 105 - protein, 738 aa, synthetic construct
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYNKSVNVDFTVDTNGVYSEPRPIGTRYLTRNL

SEQ ID NO: 106 - DNA, 2217 bp, synthetic construct
  source 1..2217: mol_type = other DNA
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggagaacagcaaacgctggaatcccgagattcagtacacttccaactacaacaaatctgttaatgtggactttactgtggacactaatggtgtgtattcagagcctcgccccattggcaccagatacctgactcgtaatctgtaa

SEQ ID NO: 107 - DNA, 397 bp, synthetic construct
  source 1..397: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 108 - DNA, 321 bp, synthetic construct
  source 1..321: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 109 - DNA, 441 bp, synthetic construct
  source 1..441: mol_type = other DNA
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaag

SEQ ID NO: 110 - DNA, 441 bp, synthetic construct
  source 1..441: mol_type = other DNA
gtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttctggttgggataaggctggattattctgagtccaagctaggcccttttgctaatcttgttcatacctcttatcttcctcccacagctcctgggcaacctgctggtctctctgctggcccatcactttggcaaag

SEQ ID NO: 111 - DNA, 1438 bp, synthetic construct
  source 1..1438: mol_type = other DNA
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctatgtcaaacatcttggagttgatatttggggaaacacaatactcagttgagttccctaggggagaaaagcacgcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttca

SEQ ID NO: 112 - DNA, 1438 bp, synthetic construct
  source 1..1438: mol_type = other DNA
ggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacattgcttttgttgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaattgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaacgcttttcatggattaggaaaaaatcattttgtctctttgtcaaacatcttggagttgatatttggggaaacacaatactcagttgagttccctaggggagaaaagcacgcttaagaattgacataaagagtaggaagttagctattgcaacatatatcactttgttttttcacaactacagtgactttttgtatttcccagaggaaggcatacagggaagaaattatcccatttggacaaacagcttgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgttggatcactttgcaaagatcctcattgagctattttcaagtgttgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcttgttgttttcttttttgctaaaactaaagaattattcttttacatttca

SEQ ID NO: 113 - DNA, 881 bp, synthetic construct
  source 1..881: mol_type = other DNA
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctc

SEQ ID NO: 114 - DNA, 881 bp, synthetic construct
  source 1..881: mol_type = other DNA
gcgggagtcgctgcgttgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttttggtaatcgtgcgagagggcgcagggacttcctttgtcccaaatctgtgcggagccgaaatctgggaggcgccgccgcaccccctctagcgggcgcggggcgaagcggtgcggcgccggcaggaaggaattgggcggggagggccttcgtgcgtcgccgcgccgccgtccccttctccctc

SEQ ID NO: 115 - DNA, 169 bp, synthetic construct
  source 1..169: mol_type = other DNA
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtgggggctagctctaga

SEQ ID NO: 116 - DNA, 2824 bp, synthetic construct
  source 1..2824: mol_type = other DNA
atgcagcgcgtgaacatgatcatggcagaatcaccaggcctcatcaccatctgccttttaggatatctactcagtgctgaatgtacaggtttgtttccttttttaaaatacattgagtatgcttgccttttagatatagaaatatctgatgctgtcttcttcactaaattttgattacatgatttgacagcaatattgaagagtctaacagccagcacgcaggttggtaagtactggttctttgttagctaggttttcttcttcttcatttttaaaactaaatagatcgacaatgcttatgatgcatttatgtttaataaacactgttcagttcatgatttggtcatgtaattcctgttagaaaacattcatctccttggtttaaaaaaattaaaagtgggaaaacaaagaaatagcagaatatagtgaaaaaaaataaccacattatttttgtttggacttaccactttgaaatcaaaatgggaaacaaaagcacaaacaatggccttatttacacaaaaagtctgattttaagatatatgacatttcaaggtttcagaagtatgtaatgaggtgtgtctctaattttttaaattatatatcttcaatttaaagttttagttaaaacataaagattaacctttcattagcaagctgttagttatcaccaaagcttttcatggattaggaaaaaatcattttgtctctatgtcaaacatcttggagttgatatttggggaaacacaatactcagttgagttccctaggggagaaaagcaagcttaagaattgacataaagagtaggaagttagctaatgcaacatatatcactttgttttttcacaactacagtgactttatgtatttcccagaggaaggcatacagggaagaaattatcccatttggacaaacagcatgttctcacaggaagcatttatcacacttacttgtcaactttctagaatcaaatctagtagctgacagtaccaggatcaggggtgccaaccctaagcacccccagaaagctgactggccctgtggttcccactccagacatgatgtcagctgtgaaatcgacgtcgctggaccataattaggcttctgttcttcaggagacatttgttcaaagtcatttgggcaaccatattctgaaaacagcccagccagggtgatggatcactttgcaaagatcctcaatgagctattttcaagtgatgacaaagtgtgaagttaaccgctcatttgagaactttctttttcatccaaagtaaattcaaatatgattagaaatctgaccttttattactggaattctcttgactaaaagtaaaattgaattttaattcctaaatctccatgtgtatacagtactgtgggaacatcacagattttggctccatgccctaaagagaaattggctttcagattatttggattaaaaacaaagactttcttaagagatgtaaaattttcatgatgttttcttttttgctaaaactaaagaattattcttttacatttcagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgaggctgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 117 - DNA, 558 bp, synthetic construct
  source 1..558: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccacc

SEQ ID NO: 118 - DNA, 437 bp, synthetic construct
  source 1..437: mol_type = other DNA
tgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagag

SEQ ID NO: 119 - DNA, 200 bp, synthetic construct
  source 1..200: mol_type = other DNA
ttaagaatatttcatagaacgaatgttccgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcaca

SEQ ID NO: 120 - DNA, 200 bp, synthetic construct; identical to SEQ ID NO: 119
  source 1..200: mol_type = other DNA

SEQ ID NO: 121 - DNA, 427 bp, synthetic construct
  source 1..427: mol_type = other DNA
taaagcaaatatttgtggttatggattaactcgaacttctagaagctgtttgcccactctatttgcccatcctaggtaggcgccctttggaccttttgcaatcctggcttctagaagagcaaacagcaaacacatcctaggtaggacttagcccctgtttgctcctccgataactggggtgaccttggttaatattcaccagcagcctcatgctagcctcgaggatatcagatctgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcgccacc

SEQ ID NO: 122 - DNA, 23 bp, synthetic construct
  source 1..23: mol_type = other DNA
gtcgagaacgttctagaacgtcg